SEQUENCE LISTING (66 sequences)

SEQ ID NO: 1 - DNA, 16 bp, synthetic construct
  source 1..16: mol_type = other DNA
  misc_feature 8..10: note = each N can be independently thymine DNA or uracil RNA
  misc_feature 13: note = each N can be independently thymine DNA or uracil RNA
ggcacgcnnnaancgg

SEQ ID NO: 2 - DNA, 16 bp, synthetic construct
  source 1..16: mol_type = other DNA
ggcacgctttaatcgg

SEQ ID NO: 3 - DNA, 16 bp, synthetic construct
  source 1..16: mol_type = other DNA
  misc_feature 1..3: note = locked nucleic acid
  misc_feature 14..16: note = locked nucleic acid
  misc_feature 4..13: note = phosphorothioated DNA backbone
ggcacgctttaatcgg

SEQ ID NO: 4 - DNA, 16 bp, synthetic construct
  source 1..16: mol_type = other DNA
  misc_feature 8..9: note = each N can be independently thymine DNA or uracil RNA
  misc_feature 15: note = each N can be independently thymine DNA or uracil RNA
gaaacgannaagganc

SEQ ID NO: 5 - DNA, 16 bp, synthetic construct
  misc_feature 15: note = RNA
  source 1..16: mol_type = other DNA
gaaacgattaaggatc

SEQ ID NO: 6 - DNA, 16 bp, synthetic construct
  misc_feature 15: note = RNA
  source 1..16: mol_type = other DNA
  misc_feature 1..3: note = locked nucleic acid
  misc_feature 4..13: note = phosphorothioated DNA backbone
  misc_feature 14..16: note = locked nucleic acid
gaaacgattaaggatc

SEQ ID NO: 7 - skipped

SEQ ID NO: 8 - DNA, 16 bp, synthetic construct
  source 1..16: mol_type = other DNA
gaattggtcatctttg

SEQ ID NO: 9 - DNA, 16 bp, synthetic construct
  source 1..16: mol_type = other DNA
  misc_feature 1..3: note = locked nucleic acid
  misc_feature 14..16: note = locked nucleic acid
  misc_feature 4..13: note = phosphorothioated DNA backbone
gaattggtcatctttg

SEQ ID NO: 10 - DNA, 20 bp, synthetic construct
  source 1..20: mol_type = other DNA
  misc_feature 9..10: note = each N can be independently thymine DNA or uracil RNA
  misc_feature 15..16: note = each N can be independently thymine DNA or uracil RNA
cagcaaacnncaccnnccca

SEQ ID NO: 11 - DNA, 20 bp, synthetic construct
  misc_feature 16: note = RNA
  source 1..20: mol_type = other DNA
cagcaaacttcaccttccca

SEQ ID NO: 12 - DNA, 20 bp, synthetic construct
  source 1..20: mol_type = other DNA
  misc_feature 1..5: note = 2'-O-methyl (2'-O-Me) modified nucleotides
  misc_feature 16..20: note = 2'-O-methyl (2'-O-Me) modified nucleotides
  misc_feature 6..15: note = phosphorothioated DNA backbone
  misc_feature 16: note = RNA
cagcaaacttcaccttccca

SEQ ID NO: 13 - DNA, 20 bp, synthetic construct
  source 1..20: mol_type = other DNA
  misc_feature 1..5: note = locked nucleic acid
  misc_feature 16..20: note = locked nucleic acid
  misc_feature 6..15: note = phosphorothioated DNA backbone
  misc_feature 16: note = RNA
cagcaaacttcaccttccca

SEQ ID NO: 14 - DNA, 20 bp, synthetic construct
  misc_feature 8: note = each N can be independently thymine DNA or uracil RNA
  source 1..20: mol_type = other DNA
  misc_feature 4: note = each N can be independently thymine DNA or uracil RNA
  misc_feature 10: note = each N can be independently thymine DNA or uracil RNA
ccanacgncngcagcacagc

SEQ ID NO: 15 - DNA, 20 bp, synthetic construct
  misc_feature 4: note = RNA
  source 1..20: mol_type = other DNA
ccatacgtctgcagcacagc

SEQ ID NO: 16 - DNA, 20 bp, synthetic construct
  misc_feature 4: note = RNA
  source 1..20: mol_type = other DNA
  misc_feature 1..5: note = 2'-O-methyl (2'-O-Me) modifed nucleotides
  misc_feature 16..20: note = 2'-O-methyl (2'-O-Me) modifed nucleotides
  misc_feature 6..15: note = phosphorothioated DNA backbone
ccatacgtctgcagcacagc

SEQ ID NO: 17 - DNA, 20 bp, synthetic construct
  source 1..20: mol_type = other DNA
  misc_feature 1: note = each N may independently be thymine DNA or uracil RNA
  misc_feature 5: note = each N may independently be thymine DNA or uracil RNA
  misc_feature 7: note = each N may independently be thymine DNA or uracil RNA
  misc_feature 13: note = each N may independently be thymine DNA or uracil RNA
  misc_feature 17: note = each N may independently be thymine DNA or uracil RNA
  misc_feature 19: note = each N may independently be thymine DNA or uracil RNA
nccgngngcaccngcgngnc

SEQ ID NO: 18 - DNA, 20 bp, synthetic construct
  misc_feature 1: note = RNA
  misc_feature 5: note = RNA
  misc_feature 17: note = RNA
  misc_feature 19: note = RNA
  source 1..20: mol_type = other DNA
tccgtgtgcacctgcgtgtc

SEQ ID NO: 19 - DNA, 20 bp, synthetic construct
  misc_feature 1: note = RNA
  misc_feature 5: note = RNA
  misc_feature 17: note = RNA
  misc_feature 19: note = RNA
  source 1..20: mol_type = other DNA
  misc_feature 1..5: note = 2'-O-methyl (2'-O-Me) modified nucleotides
  misc_feature 16..20: note = 2'-O-methyl (2'-O-Me) modified nucleotides
  misc_feature 6..15: note = phosphorothioated DNA backbone
tccgtgtgcacctgcgtgtc

SEQ ID NO: 20 - DNA, 20 bp, synthetic construct
  source 1..20: mol_type = other DNA
  misc_feature 1: note = each N may be independently thymine DNA or uracil RNA
  misc_feature 3..4: note = each N may be independently thymine DNA or uracil RNA
  misc_feature 13: note = each N may be independently thymine DNA or uracil RNA
  misc_feature 15: note = each N may be independently thymine DNA or uracil RNA
  misc_feature 18: note = each N may be independently thymine DNA or uracil RNA
ncnngagagcgangngcngg

SEQ ID NO: 21 - DNA, 20 bp, synthetic construct
  misc_feature 1: note = RNA
  misc_feature 3..4: note = RNA
  misc_feature 18: note = RNA
  source 1..20: mol_type = other DNA
tcttgagagcgatgtgctgg

SEQ ID NO: 22 - DNA, 20 bp, synthetic construct
  misc_feature 1: note = RNA
  misc_feature 3..4: note = RNA
  misc_feature 18: note = RNA
  source 1..20: mol_type = other DNA
  misc_feature 1..5: note = 2'-O-methyl (2'-O-Me) modified nucleotides
  misc_feature 16..20: note = 2'-O-methyl (2'-O-Me) modified nucleotides
  misc_feature 6..15: note = phosphorothioated DNA backbone
tcttgagagcgatgtgctgg

SEQ ID NO: 23 - DNA, 20 bp, synthetic construct
  misc_feature 1: note = RNA
  source 1..20: mol_type = other DNA
  misc_feature 6..15: note = phosphorothioated DNA backbone
  misc_feature 1..5: note = 2'-O-methoxyethylribose (MOE) modified nucleotides
  misc_feature 16..20: note = 2'-O-methoxyethylribose (MOE) modified nucleotides
  misc_feature 3..4: note = RNA
  misc_feature 18: note = RNA
tcttgagagcgatgtgctgg

SEQ ID NO: 24 - DNA, 20 bp, synthetic construct
  source 1..20: mol_type = other DNA
  misc_feature 7: note = each N may be independently thymine DNA or uracil RNA
  misc_feature 12..14: note = each N may be independently thymine DNA or uracil RNA
  misc_feature 16: note = each N may be independently thymine DNA or uracil RNA
  misc_feature 19: note = each N may be independently thymine DNA or uracil RNA
aggaggnagccnnncncgnc

SEQ ID NO: 25 - DNA, 20 bp, synthetic construct
  misc_feature 16: note = RNA
  misc_feature 19: note = RNA
  source 1..20: mol_type = other DNA
aggaggtagcctttctcgtc

SEQ ID NO: 26 - DNA, 20 bp, synthetic construct
  misc_feature 16: note = RNA
  misc_feature 19: note = RNA
  source 1..20: mol_type = other DNA
  misc_feature 1..5: note = 2'-O-methyl (2'-O-Me) modified nucleotides
  misc_feature 16..20: note = 2'-O-methyl (2'-O-Me) modified nucleotides
  misc_feature 6..15: note = phosphorothioated DNA backbone
aggaggtagcctttctcgtc

SEQ ID NO: 27 - DNA, 20 bp, synthetic construct
  source 1..20: mol_type = other DNA
  misc_feature 1: note = each N may be independently thymine DNA or uracil RNA
  misc_feature 8: note = each N may be independently thymine DNA or uracil RNA
  misc_feature 10: note = each N may be independently thymine DNA or uracil RNA
  misc_feature 12..14: note = each N may be independently thymine DNA or uracil RNA
  misc_feature 16..17: note = each N may be independently thymine DNA or uracil RNA
ncgcccancncnnngnncca

SEQ ID NO: 28 - DNA, 20 bp, synthetic construct
  misc_feature 1: note = RNA
  misc_feature 16..17: note = RNA
  source 1..20: mol_type = other DNA
tcgcccatctctttgttcca

SEQ ID NO: 29 - DNA, 20 bp, synthetic construct
  misc_feature 1: note = RNA
  misc_feature 16..17: note = RNA
  source 1..20: mol_type = other DNA
  misc_feature 1..5: note = locked nucleic acid
  misc_feature 16..20: note = locked nucleic acid
  misc_feature 6..15: note = phosphorothioated DNA backbone
tcgcccatctctttgttcca

SEQ ID NO: 30 - DNA, 16 bp, synthetic construct
  source 1..16: mol_type = other DNA
  misc_feature 4: note = each N may be independently thymine DNA or uracil RNA
  misc_feature 6: note = each N may be independently thymine DNA or uracil RNA
  misc_feature 10: note = each N may be independently thymine DNA or uracil RNA
  misc_feature 13: note = each N may be independently thymine DNA or uracil RNA
  misc_feature 16: note = each N may be independently thymine DNA or uracil RNA
cacngnaagncgncan

SEQ ID NO: 31 - DNA, 16 bp, synthetic construct
  misc_feature 16: note = RNA
  source 1..16: mol_type = other DNA
cactgtaagtcgtcat

SEQ ID NO: 32 - DNA, 16 bp, synthetic construct
  misc_feature 16: note = RNA
  source 1..16: mol_type = other DNA
  misc_feature 1..3: note = locked nucleic acid
  misc_feature 14..16: note = locked nucleic acid
  misc_feature 4..13: note = phosphorothioated DNA backbone
cactgtaagtcgtcat

SEQ ID NO: 33 - DNA, 16 bp, synthetic construct
  source 1..16: mol_type = other DNA
  misc_feature 2: note = each N may be independently thymine DNA or uracil RNA
  misc_feature 7: note = each N may be independently thymine DNA or uracil RNA
  misc_feature 12: note = each N may be independently thymine DNA or uracil RNA
cngcagnagaangacg

SEQ ID NO: 34 - DNA, 16 bp, synthetic construct
  misc_feature 2: note = RNA
  source 1..16: mol_type = other DNA
ctgcagtagaatgacg

SEQ ID NO: 35 - DNA, 16 bp, synthetic construct
  source 1..16: mol_type = other DNA
  misc_feature 4..13: note = phosphorothioated DNA backbone
  misc_feature 1..3: note = locked nucleic acid
  misc_feature 14..16: note = locked nucleic acid
  misc_feature 2: note = RNA
ctgcagtagaatgacg

SEQ ID NO: 36 - DNA, 27 bp, synthetic construct
  source 1..27: mol_type = other DNA
  misc_feature 1: note = each N may independently be thymine or deoxythymine DNA or uracil RNA
  misc_feature 4: note = each N may independently be thymine or deoxythymine DNA or uracil RNA
  misc_feature 11: note = each N may independently be thymine or deoxythymine DNA or uracil RNA
  misc_feature 13: note = each N may independently be thymine or deoxythymine DNA or uracil RNA
  misc_feature 16: note = each N may independently be thymine or deoxythymine DNA or uracil RNA
  misc_feature 26..27: note = each N may independently be thymine or deoxythymine DNA or uracil RNA
nggncagccangnaancaaacaaggnn

SEQ ID NO: 37 - DNA, 27 bp, synthetic construct
  source 1..27: mol_type = other DNA
  modified_base 26..27: mod_base = OTHER; note = deoxythymine
tggtcagccatgtaatcaaacaaggtt

SEQ ID NO: 38 - DNA, 27 bp, synthetic construct
  misc_feature 3..4: note = each N may independently be thymine or deoxythymine DNA or uracil RNA
  source 1..27: mol_type = other DNA
  misc_feature 10: note = each N may independently be thymine or deoxythymine DNA or uracil RNA
  misc_feature 17..18: note = each N may independently be thymine or deoxythymine DNA or uracil RNA
  misc_feature 20: note = each N may independently be thymine or deoxythymine DNA or uracil RNA
  misc_feature 24: note = each N may independently be thymine or deoxythymine DNA or uracil RNA
  misc_feature 26..27: note = each N may independently be thymine or deoxythymine DNA or uracil RNA
cgnncgaggnggaaganngngacnann

SEQ ID NO: 39 - DNA, 27 bp, synthetic construct
  source 1..27: mol_type = other DNA
  modified_base 26..27: mod_base = OTHER; note = deoxythymine
cgttcgaggtggaagattgtgactatt

SEQ ID NO: 40 - DNA, 26 bp, synthetic construct
  misc_feature 3: note = each N may independently be thymine or deoxythymine DNA or uracil RNA
  source 1..26: mol_type = other DNA
  misc_feature 10..11: note = each N may independently be thymine or deoxythymine DNA or uracil RNA
  misc_feature 13: note = each N may independently be thymine or deoxythymine DNA or uracil RNA
  misc_feature 17: note = each N may independently be thymine or deoxythymine DNA or uracil RNA
  misc_feature 21: note = each N may independently be thymine or deoxythymine DNA or uracil RNA
  misc_feature 24..26: note = each N may independently be thymine or deoxythymine DNA or uracil RNA
ggnggaaganngngacnacancgnnn

SEQ ID NO: 41 - DNA, 26 bp, synthetic construct
  source 1..26: mol_type = other DNA
  modified_base 25..26: mod_base = OTHER; note = deoxythymine
ggtggaagattgtgactacatcgttt

SEQ ID NO: 42 - DNA, 25270 bp, Homo sapiens
  source 1..25270: mol_type = genomic DNA
tctcagctcaatgaaccctgcctcccgggttcaagtgattctcctgcctcagcctcctgagtagctaggattacaggtgtgtaccaccatgcccagctaatttttttttttttttttttttttgtatttttagtagaaacagggtttcgctatgttggccaggctggtctcaaattcctgacctcaaatgatctgcctgccttggcctcccaaagtgctgggattacaggcatgagccaccgtgcccagccaagagggacaaaaaccttttcacagaaaaacatttttatacaacttaaccagaaagcttcatttcaggttcaggaagtggaaggggcaaaatcctggattttcacaaatttccaaaatgggcttatataactctccacccttttctttctttttttttttcttttagagacagggtctcattctgctgcccaggctgaagtgcagtggtgcaattatagctcactgtacccttgaactcctgggctcaagggatccccccatctcagcctcttaaagtgctgggattacaggtgtcagccactgctcctggctcgccaccaatgcttttcattctgatcatttctgtagtgagcagcatagaagaaataatgccttttgagggacagttgcatttaacagacagccttactaaggtgagtacgtcaggacaaagatgcgggaaggagaatcaggctattagtccattgttgcaaattgcaacgaattgagaaagtcagacacaacgatcaactgcagtgaaacatttggtggggagttttgagagtccagtctgaaggagtgcgtggaaggatcacggtccctgtgatgtaccctctctcggcttgttgctttgggcaggaatcacaagttaggaaggaatcagcaccagaaatgcagagctggctgggcgcagtggctcacgcctgtaatcccagcactttgggaggccgaggcgggaagatcacctgaggccaagagttcgagatcagcctcgccaacatggcgaagccctgtctctgataaaaaatacaaaaattagcttggtgtggtggcgcatgcctgtagtcttaactacttgggagactgaggcgggagaatcacttgaacctaggaggcggaggttgcagtgagcagagatcacgccattgcacttcagcttgggagacatcccaagaccatcttaaaaaaaaaaaaaggagggggggtgccgggcgcggtggctcacgcctataatcccggcactttgggaggccaaggcgagcgaatcacgaggtcaggagattgagaccatcctggctaacacggtgaaacctcatctctactaaaaatacaaaacattagctgagccaggtggcaggtgcctgtattcccagctactccggaggctgaggcaggagaatggcgtgaaccttggaggtggagcttgcagtgagccgagattgcaccattgcactccagcctaaaaaaaaaaaaaaaaaaaaaaaaagcttgcattgagccgagatgccaagatcacgccactgcactccagcctgggccacagagggagactctgtctcaaaaaaaaaaaaaaaaaaaaaaaaaaaaaggccgagagcggtggctcacgcctgtaatcccagcactttgggaggccgaggtgggcagatcacctgaggtcaggagttcgagacatgcctgaccaacatggtgaaaccccatctctactaaaaatacaaaattagctgggcgtggtggcacatgcctgtaatccctgctacttgggatgctgagacaggagaatcgattgaacctgggaggcggaggttgtaataagccgagatcgcaccattgcactccagcctgggcagcaagagcgagactccgtctcaaaaaaaaaaaaaaaagaaatgcagagctgaagagcagcccaatgtaaatggtcccattagagaggaactctttcatcatggacatctgtaagtgacccacactgtccagcagcatccacgatgtttgcacagcatatggccccagggagggatgtctctaatctgccagggtcctagagttaaagtcctgtgtgttgagtggagcaattttgcccatttttttagtttggcagaattcatgctgacactgaaacagctcacagcagacagcatcaggtttcagcatttttttttctctttttttaagacagggtctcactctgtcaccgaggctggagtgcagtggtgcgatcatagctcattgcagcctcgacctcttgggctcaagcaaccgtccccttgcagagctttcagcttgggatcaggtttggaaaactagaattcgtgaattcaggattttcttctgcagtagctaagccaagagactgtttgtctcgaggctaagagcgaagcgaggctttcttaagccctttaagtctgtcagtttcagagcccgggtggacgcctgtaaaaagatccctatcagctagaaaatcatgactctaagggtcagctgtgtggtttcacaagggctcattagcaactaaaaactgctttttaaaaccctgaaagtgtatctccatttagagattgcctctttttcatgtttttttccagaagctgtaatattagcaaacactatagtaagaactatcagtaactgagacttgttccattttatttacttacttacttacttatttctttattttgagacagggtctcattctgtcgcccaggctggagcgcagtggcacaatctcgcattgcaacctccacctcctgggttcaagtgattctcctgcctcagcctcccaagtagctgggtgggattacaggcacacaccaccacacccagctaattttttttttttttttgagacagagtcttactctgttgctcaggctggagtgcagtggcatgatctcggctcactgcaacctccacctcctgggttcaagcaattctcctgtctcagcctcccaagtagatgggactacaggtgcctgccaccatgcccagctaatttttgtatttttagtagagacggtgtttcaccatattggtcaggctggtctcaaactcctgacctcaggtgacccacccgcctcagcctcctaaagtgctaggattataggcatgagccactgcatccaggctaatttttgtatttttagtagagatagggtttcaccatgttggccaggctggtctcgaacacctaacctcaagtgatcctccctcctcggcctcccaaagtgctaggattacaggtgtgagccactgggccctgccttatctatttttgatacaagttctcaccctgttccccaggctagagtgcagtggcgtgatcatagctcactgcaacctttaactcctggactcaagtgatcctcccacctcagcctcctgagtggctggggactacaggtgctcactaccaggcccagctaggtgaatgcttttctgcacaccagggtattttcaaggatgcgtcctatgaacccatctgatcagtgagcttcttgagcaaggcctctggaacccacctctgcatggtcgggcatgaagaacagagacagtcacacccagccactgctgatctgttatccagtggagcccagcctgaacagagagcatcagggaggggactctggatactgggccctgcaggcttgcctccctgctctctgcccagacatgtgaatggcatctaggggtgcacccagcatgggactgagacctacagtgttggggacagagttcctacctctagatgcctgccagcctttgcaacccactgctgttgacacacttccttgagttagtaaacattggcctttgtgcttagcatataagccagggcaggctccaggagtgggggtttgagttggccacagcaatcaaagtgacaagatgttcctgctggcctggaggggataggcaggctacacagagctcttctcctccatgggaaccttctgggcctgtgagatccaccctgggccacctccactcaagttttcccagttggatatttatttatttatttgagatggagtctcactctgtcacccaggctggagggcagtggcatgatcactgctcactgcaatctctgcctccttggttcaagtgatcctcctgcctcagtcccctgagtagctgggactacagacgtgcaccaccgtgcccagctaatgtttgtatttttagtagagatgaggttttaccatgttgccaggctggtctcaaactcctggcctcaagtgatcctccctccttggcctcccaaagtgctgggattacaggcgtgagccaccacccccggcccccagttggaaatttagacttaaggagctgaggtggacactgggagcacactgggcaggcccaggacaggcagaataagggggtgggaccaccttgcctgcctcctgaatggcccagaaaatagagaaaaggagggagaagtggtctgcactggtgcaatgagatccaacttcagaatcaaacaggccagagtttggggctctgctcttaccagttccatgacctgggagacatgtgttcacctctccaggcctcagtttccccatctgatggggatatcaatatctcccatttgtatagagggcaatctagcaatttaaagtgtgggtcctggagttagaatagcccggttcatgttccagttcttccctgccgcttgctgacctcatggcctaggaatgttctctgtcccaatgaattataaccaacactgagagtgtcctgtgtgccaggtcctgttccaaagagcgtgcttatctcctttaatccccaccacactattctcattcctgttttacaaatgaggctcaaagggatatggtgattttcccaagtcacactgtatgtaacagagccaggatgcaaaccttctcctgccgtgcctctcagagctatggaatacaacgctgtgacgcaaacaggactttggggccagtctccaaatgccccctcaaatttcctctaggcaagagttctggagccgccactgcactccagccggccttggcttctgagctttggattctgggcagtaatggtcacattcccagcgtcctggaaaagcttgcccttggcttctggccctgactctgcctcccactgactagtggacgtcactgcccctctctgggcctggcaacaccatcctgttcaatgaggctgcggctggatcagaggtccccaggcctggaaccttccagggctgggctcagctccccaccccatcccccaggtaggccaggaggcctgggcgggacctgcttggggttaaatactccggcccagcactccccaggcctctagtcccagtaggaggtttgactaagatcaatcatggtaagtgccatggccatctccccttgggtgggacatcttcagtaacgcttccagaagcaccttgtgttggaacctccaagtctcactgtccttctttttctccttagacgacttacagtgacaaaggggcaaaggtaagcaggcgaaatgtgggaaatgtaggggcctgagtctagaagggggtcggagtgcagggagactggggtttcagcagagagcagaagaggaaggactggagtggatacatgacgtgcatgcctgcacactcagctttacccccggggctccccgagactgagctggactcaacctcaccctcacaacaaacttgtatcctggccgggcatggtggctcatgcctgtaatcccagtgctttgggaggccaaggcaggcaaatacttgaggtcaggagttcgagaccagcctggtcaacatgaggaaaccccgtctctactaaaaatacgaaaattagccgggcatggtagcgcacccctgtaatcccagctactcaggaggctaaggtgggggagatcacttgaaccctaggatcgcttgaacctgggaggcggaagttgcagtgagccaagattgcaccactgcactccactccagcctgggcaatagagcaaaactccatctcagaaacaaaaaccaaaacaaaacctgtaccccttctcaaatgcctgccaatcttttaatcccatctggaagttctttctaaggtctaacttaaatccatattgctgcaacataagtctatttccctgtgtgaaaccttggcaggcctcctgagggggtttccccatccaagacacccaggagctcaacttccacatgagaaacctcttttgaggcttggcaaggctgcatggggttccgctgctcctaagttctgtggttgaggctggaataaggagggtggctaggggacatcactccacccgcacttttctagactcactttcagggactcaacctcacctcttctctccttctgcagcctgagagaggccgattcctccacttccactctgtgaccttctgggttggcaacgccaagcaggtagaagccggggcaaggtgctggagtgagcagacggcagggcagagggctgcctgacaagggcaggcctccagcagggtctgtgattggcccttgggagggaggatcaggccaggggtgggccagatactctgaatcctaattacaccagcgggaaagttatctgtcagcaaaatgtgcaggcagcagtctatcttcccaggagggtctctgcccctgaggcaatagggcaggcaggctgaggggggccaggggctctcttgcggaagtcccccatgctggccccttgaccctcatgaaccctgctgcaaccctgtgattcaggccacgtcattctactgcagcaagatgggctttgaacctctagcctacaggggcctggagaccggttcccgggaggtggtcagccatgtaatcaaacaagggaaggtgagtcaccacccccaggacagcccccaacccctgcctggccaacatggtgaaaccccatctctattaaaaatagaaaaattagctgggcatgatggtgggcacctgtaatcccagctactcaggaggctgaggcaggcacatcgcttgaacccgggaggcagaggttgcagtgagccaacgtcgtgccactgcactccagcctgggcgacagagcaagactccatctcaacaaaaaaaaacaaaaaacaacaaaaaaccccaacaacaacaacaaaaacatataattcagctcccaccgacccctccgctggcatctttgtacagccatgcaacacggcccttcctagtttcgtttcctcctcagcctggcccaacactgaccccacaggtgctcagaaaacctgggatatcagaggagaggaattttaagcagtgccctgaccgatggggtccctggagttctctccccgagggagacctgacctagcagttcctctgaggatccagagggggtctggctgaaagccccatccctcaggaccagtgtgggaaactaaggcagagaaggagctagaacccaggtccaggagttcttgtgctccatcaggggtgtttcgggtcggcagggaggggccgatgggtgcacctccccatgcccccctgagccactagcctcacctccttctcctcctatcttagattgtgtttgtcctctcctcagcgctcaacccctggaacaaaggtgaggtgcccggggagggtggacgcatggctctgtgggtgggatctgggttctgggagggtggacgcatggctccgtgggtgggctctgggttcagggcctggatggagactaaaagccaggtgaggggacctagactccagactccagtgccatcctctccctgccggccacagagatgggcgatcacctggtgaaacacggtgacggagtgaaggacattgcgttcgaggtggaagattgtgactacatcgtgcaggtaagaaagcacctgggtggactgagagaggaaggcctccgtctgccatggggaccccatcagtcagggctggaaggacactgctgcatcacagtgacaagcccagatgccacatcacagtgacaagctcagatgctgcatcacagtgacaagcccagatgctgcatcacagtgacaagcccagatgctggtgtgacacacacctgggctccgggggtgccctgccttcccccgtgtcctctctctcactgagcctcagtttccacagctgccagatggggctaaccacagctgggttgtcaaaggaggaaattagaattaccatttccttttttttttttttttttttttgtgagacggagttttgctcttgttgcccaggttggagtgcaatggcacgatctcggctcaccgcaacttctgcctcctggattcaagcaattctcctgcctcagcctcctaagtagctaggattacaggcatgcgccaccacgcccggctagttttgtatttttagtagagatggggtttctccttgttggtcaggctggtcatttccttttttttcttttttcattttctttttctttcttttgtgtgtgtgtgtgtgtcagagtcttgctctgttgcccaggctggagagcagtggcgcaatcttggctcactgcaacctccacctcctgggttcaagcgattctcctgcctcagcctcccgagtagctgggattacaggcgcctgccaccatgcccagctaatttttgtattttagtagagatggagttttgccatattggccaggctggtcttgaactcctgacctcaagtgatccacccacctcggcctcctaaagtgctgggattacaggcatgagccattttgcctggcctcagaattactgtttcttgagcacaactctaaaccaaacactgtacccaacaggcagcctacagattcagagtgtgcgttccagagccaggctctccaggtctgaatcttagccgtgtgacttcgggcaagtgacttaatgtctctgtgcttcaacgtcctcacctagagaatggagatgataatagtacttttgcataatgttgctgtgaggatgaacgaggttagaatctagaacagagcctggtgcatgatataagcactgtcatgcagtccagttttgctaaataaatgcatgaatcttcttattattctcatccaaccattctttttacagatggtgaaattgaggactagagaaggggactgatcacagggggagttagcagaggcaggacagacacccatccctacctagtttgtctcccatagccccaactctgggcctgggcctcagtttccctatttgccaaatgagcatgagtattgggggtaatggctgaagctggctggggttcctacctggggctggagaaactggaggggcctgcaggaccacgcagaggccaccaggcaggagcagcaggcaagggttctggaaggctccagccctcaccaattccccgatcccatcttctctgcagaaagcacgggaacggggcgccaaaatcatgcgggagccctgggtagagcaagacaagtttgggaaggtgaagtttgctgtgctgcagacggtgagttccctttggtgcccccacccctcctgctgtcggcaccactgagatgccaggagccccgcagctgactccatcaaaccatcccacccatgggccttggagcacagacattgtccctcaagtctgtcccccgtattgcctggccagtgtacggggactgcaggagccaccagccactcctcccaggcttcccttcctcccagtgcgggaggatgacagcaagggggtgaaggcaggggctctgaagaagggagaaggatggggtctggggacatcagttcccacacgagctgtgtgactttgggcatattactgtacctctctatgactctgtttaaatgagcaaagactggaactggaacacctatgtggtctgggacatgcgcttaactttcctgtgcctcagcagtatctttttgttttcctcaaaaaaatttttttttaaagatggagtctcgctctgtcgcccaggctggagtgtagtggtgtgatcttggctctctgcaacctccgcctcccaggttcaagtgactctcctgcctcagcctcccgagtagttgggaccacaagtgcgtgccaccatgcccggctaattttttcttttttgtatttttagtagagatggggtttcaccgtgttagccaggatggtctcaatctcctgacctggtgatccgcccaactcagcctcccaaagtgctgggattacaggcgtgggccactgtgcccggcctttttcttttttttttattcttttttcttttttcagacggagtctctctgtcgcccaggctggagtgcagtcgcgtgatctcagctcactgcaacctctgcctcctgggctcaagtgattcttctgtctcagcctcctggggattacaggcatccacccgcacacacggctaatttttgtgtttttagtagagacagggttttgccatgttggccaggctggtctcgaactcctgacctcaagtgatccatccgcctcagcctcccaaagtgctgggattacaggcgtgagccaccgtgccaggctgttttcttcaactttcttttttctttttttttttttttttgagatggagtctcgctctgtcgcccaggctggagtgcagtggcacgatctcggctcactgcaagcgctgccttccgggttcacgccattctcctgcctcagcctcccgagtagctgggactacaggcgcccgccaccacgcccggctaaattttgtatttttagtagaggtttcaccgtgttatccaggatggtctcgatctcctgaccttgtgatccgcccgcctcagcctcccaaagtgctgggattataagtgtgagccaccgcgccccacctcttcaacttttattataggttctggggtatatgtgctggatgttcaagtttgttacataggtaaacgcatgcatggtggtttgctacacagatcatcccatcacctaggtattaagcccggcatctgttagctactctccttggtgcactccctccccgacctcccccacctgacaggccctagtgtctgttgttccccgtgatgtgtccatcttttctcatcatttagctctcacttctaagtgagaacatgcagtgtttggttttctgttcctgcgttagtttgctgaggataacggcttccagctccatccatgtccccgtaaaggacatgatcttgttcctttttatggctgcaagcctcagtatcttcaactatgactcctcatagggtcatcgtaaggatccaatgagaactgggcgcagtggctcacgcccataatcccagcactgtaggaggctgaggcgggcgaatcacttgaggtcagaagttagcctggccaacatggtgaaaccccacctctactaaaaaaaaatacaaaagttagccaggcatggtggtgtgcacctgtagtcccagctacttgggaggctgaggtgggagaatcgcttgaacccaggaggcagaggttgcagtgagccaagattgcacctctgcactccagcctgggcgacagaacaagactccctctcaaaaaaaaaaaaaaaaaaaaaaaaaaaaaaggatccaatgaggtcatttgtgtatagcacttagaacagtgcctggcacattagacactcagcaaagatgaccaattctgtttaggacccgtgttactgtaatacagagacaatcatagctcttacctccttgagattaaattcaacataaagggctgggcacagtggctcacacctataatcccagcagtttgggaggctgaggcaggaggatcacttgaggccaggagttcgagaccagcatgggcaagatagggagaccccatctctacaaaaattaaaaatacaaataaaaaataaataggccaggtgcagtggctcacacctgtaatctcagcaatttgggaggccaaggcaagaggactgcttgagcccaggaatttgagaccagcctgcgcaacaaagtgagaccccatctctacgaaaaaattaaaaattggccaggcatgatggagcatgcctgtggtcccacctacatgggaggctgaggtgggaggattgcttgagtccaggaggtagaggctgcagtgagctgttatcacaccactgcacttcagcctggttgacagagtgagactctgtctcaaaaataaatgaataggccgggcgcggtggctcacgcctgtaatcccagcactttgggaggccgaggcgggcggatcacgaggtcaggagatcgagaccatcctggctaacacggtgaaaccccgtctctactaaaaaaaaaaaaaaaaaaatacaaaaaagtagccgggcgtggtagcgggcgcctgtagtcccagctactcgggaggctgaggcaggagaatggcgtgaacccgggaggcggagcttgcagtgagccgagatcgcgccactgcactccagcctgggcgacagagcgagactccgtctcaaaaaaaaaaaaaaaaaaaaaaaaaaaataaataaataaataaatgaataaaaatgttttaaaaagttaaaaaattaaattaaacataaaaatggttaaagtgtgcctgtaatcccagcactttgtgaggccgaggcaggtggatcacctgagttcaggagttcgagaccagcctggccaacgtggcaaaaccctgtctttactaaatatacaaaaattagctgggcatgacaggcgcctgtaatcccagctactcgggaggctgaggcagaagaatcacttgaacccgggagacagaggttgcagtgagccaaggttgcaccactgtactccagcctgggcgacagagcgagactccatctcaaataaataaataaaaattttaaaaaataaaagttaaaaaattaaattaaacataaaaatggttaaagtgtgcctgtaatcccagcactttgggaggccaaggtgggtggatcacttgaggtcaggagttcaagaccagcctggccaaaatggtaaaaccccatgtcactaagtaaaacaacacaaaaattagccaggtgtggggctgggcacggtggctcatgcttgtaatcccagcactttggaaggccaagggggcggatcacctgaggttgggagtttgagaccagcctgaccaacatggagaaaccccatctctactaaaaatacaaaattggctggacgtggtggtgcatgcctgtaatcccagctactcaggaggctgaggcaggagaatcgcttgaacccgggaggcagacgttgtggtgagccaagatcgcaccattgcactccagcctgggcaacaagagcggagctctgtctcaaaaaaaaaaaaaaattagccaggtgtggtggcacgtgcttgtagtcccagctatttgggaggctgaggcaggagaatcgcttgaacctgggaggcggaggttgcagagagccaagatcacaccactacactctagcctggccaacaagcaagactccgtctcaaacaaaaaaaaaaaaaaaaaaaaaggctaggtacagtggctcatgcctgtaatcccggcactttgggatgttgaggtgggcccggcatggtggtacacgcctataatcccagctactcaggaggctgaggcaggagaatggcttgaacccgggagatggaggttgcagtaagccgagatcgtgccaatgcactccagcctgagtgacagagtgagactccgccttaaaaaaaaaaaaaaaaaaagatggttaaagtggctgacagaactcttcagtataattttttttttgagacggagtctcattctgtcgcccaggctggagtgtagtggcacgatctctgctcactgcaagctctgcctcctgggttcatgccattctcctgtctcagcctcccaagtagctgggactacgggcacccgccacgccaccacacccggctaattttttgtattttttagtagagacaggatttcaccgtgttcgccaggatggtctcgatctcctgacctcatgatccacccgccttggcctcccaaagtgctgggattacaggcctaagccaccgcgccaggctgctcttcagtataatttaatccaatgccctcctgcgttcttcatctagttgacctactgaaataaatgaagtaggtgcttcattgagtacctcccctgtgccaggcactgagcttggcgctgggtacaccatcatgagcaaaacagtgtccccgccctcacggaaactctcaaacaagtgacagattgcaattcatttcagaaaggagaagtgacttacccaagatcacgcatctggttagggtcagatgcaggttggaacccggggctacgttagcgctatgaaatgtgctctgtcctcggcggggatgggtgcccaggggccgagccaggctgttttccacccgtagtatggggacaccacacacaccctggtggagaagatgaactacatcggccaattcttgcctggatatgaggccccagcgttcatggaccccctacttcctaaactgtgagtgtccctcagggcagaggtgggtggaaagccctgagggactgtgaaaagtaatgccccttcctccatgtccactgcgcatgtgtgttggggcagaatggagccggctagggaattcctggaagtcctcaggggtgcttgaagaggtggcacagaggctgagaccatggcctctgcaaccaaacttccccgggttcaaatcccagctctgttacttcacagccatttcaccctgggcaagtcgcttcatctctctaggcctcagtttcttcatctgtaaaatggggactgtaatagtaaatgagttgacaaaataaaatgtccggcccattagtaaatgctacataagtgtgagtccaggcgcagtggctcatacctataatcccagcactttgggagtctgaagcaggaggttcacttgagccccagagttttagaccaacctgggcaaaaaaaagagtctctctttttgtagagactccatctctacaaaaaaatagaactccaaaaacagaagtgtgagctattattattattgaggggtcatggagaagggagcaatctggggggtagccaccttctcacccccatcttcttcccttcccaggcccaaatgcagtctggagatgatcgaccacattgtgggaaaccagcctgatcaggagatggtgtccgcctccgaatggtgagaccttggccctctccctctgcgtgaagatgacgggtcctcacggtggactggcccactccctggtccttgcactaacatggaaaatacgactgggtgcagtggctcacacccgtaatcccagcattctgggaagccgaggtgggaggatcacttgaggtcaggggttcaacaccagcctgaccaatatggagaaaccccgtctctactaaaaatataaaaactcagtgggcgaggtgttgggcccctgtaattccagttactcaagaggctgaggcaggagaatcgcttgaacccgggaggtggaggttgggcgacagagtgagactccagcctgggaaacagagcaagattttgtctccaataaacaaataaaataaaatagaccgggcgcacttgctcacgcctgtaatcccagcactttgggaggcggaggcgggtggatcacctgaggtcaggagttcaagaccagcctgaccaacatggcgaaccccggtctctactaaaaatacaaaaattagctgggtgtggcagtgtgtgcctgtaatcccagctacttgggaagctgagacaggagaatcgcttgaaccaggaaggcggaggttgcagtgagctgagattacgccactgcactccagcctgggtgagagagagagactttgtctcaaaataataataataataataaagtaaaataaaataaaagtggaaaatacagcacaagatgttgggagttagcctgaggccactgatgcatttattcactgtaaaactgtcaacatggagaccagtggacttgaggcccaaagcgacccacaggccctccttctgaggaaccctggggacatcacagtccagggaggcttggaggaaggttgccaccacccagagagccccctgtccaaattccaagcaagggcctttttctcaaagcactttacttccatctgtgggagccttgccgtcctgtatgtatttcgttccaagacacttccacctgaaggaaaaatgtcatcataaaggtacaaatctaggctgggcgtggtggcccatatttggaatcttagtactttggaggcccaggcaggtggatggcttgaacccaggagttcaagacagcctgggcaacatagtgagaccccatctctacaaaaaatacggccgggcacagtggctcatgcctgtaatcccagcactttgggaggccgaggcgggtggatcacctgaggttaggagttcgagaccagcctgaccaacacagtgaaacactgtctctactaaatacaaaaaattagttgggcatggtggtgcatgcctgtaatcccagctacttgggaggctgaggcaggagaatcgcctggacccaggaagtggcagctgcagcgagccgagattgcgtcactgcattccagcctgggcgaaaagagcgaaactccgtctcaaaaaacaaaaaataaaaataccccaaaattatccaggtgtggtagcacatgcctgtagtcccagctacttgggaagctgaggtgggaggagcaattgagcctggagattgaggctgcagtgagccaagactgcaccactgcactccagcctgggcaagagtgagactctgtctcgagcaacaacaacaacaaaaagatctaaatctctgatgggtatgatggcagtggcatctcctttctggagcctgctcagggacagattctggaaggtggaaatgggtgatggaggccgtccctggaggcgctcagagcatatgtgttccaccgcccaccctacaggtacctgaaaaacctgcagttccaccgcttctggtccgtggatgacacgcaggtgcacacggaatatagctctctgcgatccattgtggtggccaactatgaagagtccatcaagatgcccatcaatgagccagcgcctggcaagaagaagtcccagatccaggtgaggccgcccctggctgggggagaggggagcagggaagaggtctgggggctcttgtagggtcagggatgtgggtgatggggcaaatgacagtggtctccttgtttccctcccccaggaatatgtggactataacgggggcgctggggtccagcacatcgctctcaagaccgaagacatcatcacagcggttagaacccccttccctgtccgaattcctctctcattcagagccagggacgatctctgcaaaattgagagggcgggtggcggcagtcctgggcccgtttctctgggctgggagactgacaggactccctcactgcttcttgcaactcatgggcaggcacagccacacccacccccagagagttcagtctccacagaacccctgggctttggaggtagcaggcgccagggtatcaggaagcctgggtgatggtcccggttctgccagtggcttgctctgtgtctctgggagattcagtgctgtctctggactttagttgccttaccttaaaaatcaggaaattagggccgggcacagtggctcacacctgtaatcccagcactttgggtggccaaggcagggggatcacctgaggtcaggagtttgagaccagcctggccaacatggtgaaaccccatctctactaaaaatacaaaaattatctgggcgtggttgcgggcacctgtaatcccagcttctcaggaggctgaggcaagagaagtgcttgaactcaggaggtagaggttgcagtgagccaaggtcgcaccactgtaccacagcctgggcgacaagagtgaaattctgctttagaaaaaaataatagtaattaataaaggaaattaggactgggcacagtggcttatgcctgtagtcccaactacacgggaggccaaagcagaaggattgtttgagcccataagttagagatcagcctggaaaacagtgggaccctgtctctgcaaaaagtaaacaaaattaaccaggcttggtggttcatgcctgtagtcctagctacttgggaggctgaagcaggaggatccatttagccaaggatttcaaggctgcagttagctatgattgagccacttttctccagcctaggtgacagagagtgaggccctatctccccgcaacctcaaaaaagaaattttttttaaaatgaggaaattaaggttgggcatggtggctcatgcctataacagtttctgcccttgtggagcaataaacaaaataagtaaaatataaatgctagatggtagagagcacccaggaagaaagtaaagcagggaattgggacagcaagggtgaatggcattgcacatttaaattggggggtcagacactgtgggaggccgaggcaggaggactgcttgagctcagaagttcaagaccaacgtgggcaacatagtaagacctcatccttttttttttttttttttttgagacggagcctggctctgtcgcccaggctggagtgcagtggcgcgatctcggctcactgcaagctccgcctcctgggttcacgccattctcctgcctcagcctcccaagtagctgggactacaggcacccgccaccacgcccagctaatgtttttttttgtatttttagtagagacggggtttcactgtgttagccaggatggtctcgatctcctgatcttgtgatctgcccaccttggcctcccaaagtgctgggattacaggtgtgagccactgcgcccggccataagacctcatctttacaaaaaaatcaaaaaattagccaggtgtggtggtgcacacctgtggttctagttacccagaagtctgaagtaggaggatcagctgagcctggggaggttgaggctgcagtgagctaagatcatgccactgcactgcagcctgggtgacagaacaaagaccctgtctctcaaatatatatatgtgtgtgtatatatatatatatccttcttttttgagatgcagtcttgctctgtcttctaggctggagtgcagtggtgcgattttggctcactgcaacctccacctcctgggttcaagcgattctcctgcctcagcctcccaagtagctgggattacaggtgcatgacaccacgcctggataactttttttgtattttatttttattttttattttttatttattttattttattttgagacagagtctcgctctgttgctcaggctagattgcagtgacgcgatctcggctcactgcaagctctgcctcccgggtttacgccattctcctgcctcagcctcccaagtagctgggactacacgctcccaccaccacgcccagctaattttttttttgtatttttagtaggggtttcactgtgttagccaggatggtcttgatctcctaacctcgtgatcacccatcttggcctcccaatgtgctgggattacaggcgtgagccacctcgcccggccttttttttgtatttttaatagagacaggattttgccatgttggtcaggctggtctccaactcctgacttcaagtgatctgcccgcctcagcctccaaaagtgctgggcttacaggcgtgagccaccacactcggcctaatagatccttaatcgtttcatgcctcagtttccccagctatgaaatgggagaatagtccttcagctgcctcaaaagattgctgcatggacataatgagggaatacgtagaaagtgctcaggccgggctcagtggctcacgcctgtaatcccagcactttgggaggccgaggtaggtggatcgcctgaggtcaggagttcgagaccaacctggctaacatggtgaaaccccatctctactaaaaatacatagctgagtgtgatggtagtcacctgtaatcccagctactcaggaggctgaagcaggagaatcgcttgaaccgggaggcagaggttgcagtgagctaagatcgtgccactgcactccagcctgggcaacagagactccatctcaaaaaaaaagaaaaaaagaaagtgctcaaggcaggcacatggtgagtgttctataaatctagcagttgttgttctaacggggcccatgacagagaagaaaggggaagaggccacacagcatcctggcagttgaggggagctaggatagggaccctgtatcctgatgacccagcacctccacctggctaggggagttgaacttggaggccgaaggctgacctcagctttgctctgaaacagattcgccacttgagagagagaggcctggagttcttatctgttccctccacgtactacaaacaactgcgggagaagctgaagacggccaagatcaaggtgaaggagaacattgatgccctggaggtgaggcccaggcaggatccgcagccttgtggggggagtatgagctctgcattgcatcttttcagaaatattgtctcagcccggaggggagcccagaccaagtccctgctggcaaaggacatttattctagggtctatgtgggaggaggagacagatgacaaacaaataaacaataaaagcacggtcagagatagtcagtgcaggaagttcaaacaggcagcctggggctgctttacagagactggtcagggagggcctctctgaggaggtgacatctgagtgaagtctagatcatgagcgggagctggccatgtgaacagcgggaagaaaggcattcctggcagagggatcagcaagggccgaggtaggagcatgccctgagcgtctgaggaacagaaagaataggctagagtggatgggaggctgtttttgagcgatgacctaggtcagacaggtaagcaagagccagttcctcttgtgagctattattcaggaatctggattttgttcttttgtgaccagaaaccactggagggttttttggtttggttttagcagtgttattgacatatgtcacataacatgtcatttttttaaggtgtacaattctatgatttctagctatttgctatgttgtgcaactatcatcaaaatccattttaggactgggtgtggtggttcatgcttgtaatctcagtactttgggaggccgaggcgggcggatcatgaggtcaggagttcgagaccagcctgaccaacatggtgaaaccctgcctctactaaaaatacaaaaattagccaggcgtggtgacacatgcctgtaatcccagctactcaggaggctgaggcaggagaatcacttgaacccgggaggcggaggttgcagtgagccgagattgcgctactacattccagcctgggcgacacagtgagactctgtctcaaaaaaaaaaaaaaaaaaaaaaaagatccattttaggactaggtatggtggttcatgcctgtaatcccagcactttgggaggccaaggcaggtggatcacttgaggtcaggagtttgagaccagcctggtcaatatggcgaaacccccatctctacaaaaagaaaaaaaaaaattagctgtgcgtggtgatctgtgtctgtggtcccagctactggggaggctgaagtgggagaatcacttcaacccaggaggtagaggctacagtgagccatcatcaggccactgcactccagcctgggtgataccctgtctaaaagaaaaacaaaaacagttaggccagacacagtggctcacacctataatcccagcattttgggaggttgaagtgagaggattgcttgaggccaggagtttaagaccagcctgagaaacacagggcgaccctgtatctacaaaaaaaacagttttaaaaaattagctgggcatggtggttcgtgcctgtagtcccagctactcaggaggctgaggtgagaggatcacttgaggccaggaggtcaaagctgcagtgagccatgatcaggctactgcattccagcctgggcaaaaaatcaattttagaacatttttgtcaccccaaaaagaaaccttgagcctcttagccagcacctcccaaccccctctgttcccttcccccctcccaccccagccctaggcaccattaatctaccttcagtctctatagatttgcctattctggacatttcatcaaaaggaatcacaaagtccaggcacggtggctcatgcctgtaatcccagcactttgggaggctgaggtgggcggatcatgaggtcaggagttcaagaccagcctggccaacatggtgaaaccctgtctctactaaaaatacaaaaattagctgggtgtgatagcagatgcctgtaatcccagctactggggagcctgaggccggagaattgcttgaacccagggaaggggaggttgcagtgagccgagatcgcgccattgcactccagcctgggcgacagagtgaaactccatctcggaaaaaaaaaaaaaggaatcacaaaataggcctattgtgagcagcttctttcccatagcatgtgctcaaggttcatctgcgtggggtgtgtgtcagtgcttcgttcctttttgtggctgaaaaatatcccattgtatagatagacacacattgtttatctattcctcgttgatagatatttgggttgtttccacttttggggtgttatacgtaatgctactgtgaacatctatgtacaaggctctgtgtggacgtgttttcaatgcactttggaatatatctaggagtagctaggtcacatggtaattccatgttcccttttgtttgtttagagatggagtcttgctttgtttcccaggctggggtgcagtggtgcgatcttggcccattgcaacctccagcctcctgggttcaagcaattctcctgcctcagcctccccagtagctgggattataggcgcccgccaccacgcccagctaatttttttttttttttttttgagacagagtcttgctctgttgcccaggctggagagcagtggccgatcttggctcactgcaacctccacttcccgggttcaagcgattctcctgcctctgcctcctgagtagctgggataacaggcgtgtgccaccatgcccagccaatttatttttatttttatttttagtagagatggggtttcaccatgttggccaggctggtctcgaactcctgacctcgggtgattcactcacctcagcctcccagagtgctgggattacaggcgtgagccaccttgcccagcctaatttttgtatttttttttgtagtgactgggtttccccatgttggccaggctggtctcgaactgagctcaggtgatccacctgcctcggcctcccaaagtgagccaccatgcctggccacccccttttttttttttaatccgtattttaaggaagcaacactatatgtttaactttttgagaagctgccagactgttttccaaagtggttgcaccattttacagtcccaccagcagtacatgagggttccgatttccactggaggattttaaacaggggaatgacatgatccgacttttaaaagatcccgcagcaggtcatggtggctcacgcctgtaatcccaacactttgggaggtcgaggtgggtggatcacctgaggtcaggagttcgagaccagcctgaccaatatcgtgaaaccccgtcttactaaaaatacaaaaattagccgggcatggtagcctgcgcctgtagtcccagctactcgggaggctgagacaggagaatcgcttgaacccggaaggcggaggtttcagtgagccaagattgcgccaccgcactccagcctgggtaacagcgcaaggctccgtctcaaaaaagaaaaagaaagtgtcctgataatgtctgcggggaggatggattgtaaggatacaagattagaagcgggcagcagtcttgaatggtgccttaggaactgtagtagggggatagtaataggacagtttggggatatatttggaggtggctgaactgccagaaaaagaggtcatttccagacttttggggtgcaggagcccctgtggactttccaaggatctcaccgtgccagcctcccctagcagagccccctgtctcccgcctaggagctgaaaatcctggtggactacgacgagaaaggctacctcctgcagatcttcaccaaaccggtgcaggaccggcccacgctcttcctggaagtcatccagcgccacaaccaccaggtactgcttgtccccggcaggcccgaggggacaggcagctagcacactcggttttctgggtccttggctcatctcctctctttcccgccacagggttttggagccggcaacttcaactcactgttcaaggctttcgaggaggagcagaacctgcggggtaacctcaccaacatggagaccaatggggtggtgcccggcatgtaagccccgcccaccccacggaggccacagccacacagccacgccccctgattctggaactcgcccaacttccctactggctgctccccttgggtcccgcccaccagcggactcggcccccaaggctccgcccacactgaccacgcccctcggcggggccgccctctgctccagccctcccgattaaagcgtgccccggtccaatactgtgtctgtgtttgtgttgcgcataaggcagtagtgccttatggcagccagggggaggccagggcggccgccagcgccgcgggaccgaggagctgcgggtgccagcgctttgccagcgggaccacgcggagaggggaattctggaaccagggaaggaaggggacttgctctggagggaaagacggatgacactgcttttgtagagagataaacgcgattcagacaataaagcaagcgatgagccgggcgtggtggctcacacctgtaatttcagcgctttgggaggcagaggtgggaggatcgcttgaggccaggagttcgagcccagcccggtaacatagcaagaccccgacccccctgcgtctacacaaataacaaataaaaattagcctggcgtagtggcgcacacctgtggtcccagctactcaggaggctgaggtgggaggatcgattgatggacagaggtctaagctgcagcgagccctgatcctgacactgcactccagcctgggcaagagtgagaccctgtctcaaaaaaaaaaaaggcagttgatgagaatatgtggaagcgaagatcgcaggggagctaattttgat

SEQ ID NO: 43 - DNA, 1419 bp, Homo sapiens
  source 1..1419: mol_type = genomic DNA
ctctagtcccagtaggaggtttgactaagatcaatcatgacgacttacagtgacaaaggggcaaagcctgagagaggccgattcctccacttccactctgtgaccttctgggttggcaacgccaagcaggccacgtcattctactgcagcaagatgggctttgaacctctagcctacaggggcctggagaccggttcccgggaggtggtcagccatgtaatcaaacaagggaagattgtgtttgtcctctcctcagcgctcaacccctggaacaaagagatgggcgatcacctggtgaaacacggtgacggagtgaaggacattgcgttcgaggtggaagattgtgactacatcgtgcagaaagcacgggaacggggcgccaaaatcatgcgggagccctgggtagagcaagacaagtttgggaaggtgaagtttgctgtgctgcagacgtatggggacaccacacacaccctggtggagaagatgaactacatcggccaattcttgcctggatatgaggccccagcgttcatggaccccctacttcctaaactgcccaaatgcagtctggagatgatcgaccacattgtgggaaaccagcctgatcaggagatggtgtccgcctccgaatggtacctgaaaaacctgcagttccaccgcttctggtccgtggatgacacgcaggtgcacacggaatatagctctctgcgatccattgtggtggccaactatgaagagtccatcaagatgcccatcaatgagccagcgcctggcaagaagaagtcccagatccaggaatatgtggactataacgggggcgctggggtccagcacatcgctctcaagaccgaagacatcatcacagcgattcgccacttgagagagagaggcctggagttcttatctgttccctccacgtactacaaacaactgcgggagaagctgaagacggccaagatcaaggtgaaggagaacattgatgccctggaggagctgaaaatcctggtggactacgacgagaaaggctacctcctgcagatcttcaccaaaccggtgcaggaccggcccacgctcttcctggaagtcatccagcgccacaaccaccagggttttggagccggcaacttcaactcactgttcaaggctttcgaggaggagcagaacctgcggggtaacctcaccaacatggagaccaatggggtggtgcccggcatgtaagccccgcccaccccacggaggccacagccacacagccacgccccctgattctggaactcgcccaacttccctactggctgctccccttgggtcccgcccaccagcggactcggcccccaaggctccgcccacactgaccacgcccctcggcggggccgccctctgctccagccctcccgattaaagcgtgccccggtccaa

SEQ ID NO: 44 - protein, 393 aa, Homo sapiens
MTTYSDKGAKPERGRFLHFHSVTFWVGNAKQATSFYCSKMGFEPLAYRGLETGSREVVSHVIKQGKIVFVLSSALNPWNKEMGDHLVKHGDGVKDIAFEVEDCDYIVQKARERGAKIMREPWVEQDKFGKVKFAVLQTYGDTTHTLVEKMNYIGQFLPGYEAPAFMDPLLPKLPKCSLEMIDHIVGNQPDQEMVSASEWYLKNLQFHRFWSVDDTQVHTEYSSLRSIVVANYEESIKMPINEPAPGKKKSQIQEYVDYNGGAGVQHIALKTEDIITAIRHLRERGLEFLSVPSTYYKQLREKLKTAKIKVKENIDALEELKILVDYDEKGYLLQIFTKPVQDRPTLFLEVIQRHNHQGFGAGNFNSLFKAFEEEQNLRGNLTNMETNGVVPGM

SEQ ID NO: 45 - DNA, 17 bp, synthetic construct
  source 1..17: mol_type = other DNA
  misc_feature 1..2: note = each N may be independently thymine DNA or uracil RNA
  misc_feature 4: note = each N may be independently thymine DNA or uracil RNA
  misc_feature 7: note = each N may be independently thymine DNA or uracil RNA
  misc_feature 15..16: note = each N may be independently thymine DNA or uracil RNA
  misc_feature 13: note = each N may be independently thymine DNA or uracil RNA
nnanagnccacanannc

SEQ ID NO: 46 - DNA, 17 bp, synthetic construct
  misc_feature 1..2: note = RNA
  source 1..17: mol_type = other DNA
  misc_feature 4: note = RNA
  misc_feature 15..16: note = RNA
ttatagtccacatattc

SEQ ID NO: 47 - DNA, 17 bp, synthetic construct
  misc_feature 1..2: note = RNA
  source 1..17: mol_type = other DNA
  misc_feature 4: note = RNA
  misc_feature 15..16: note = RNA
  misc_feature 1..4: note = 2'-O-methoxyethylribose (MOE) modified nucleotides
  misc_feature 14..17: note = 2'-O-methoxyethylribose (MOE) modified nucleotides
  misc_feature 5..13: note = phosphorothioated DNA backbone
ttatagtccacatattc

SEQ ID NO: 48 - DNA, 20 bp, synthetic construct
  source 1..20: mol_type = other DNA
  misc_feature 1..2: note = each N may independently be thymine DNA or uracil RNA
  misc_feature 11: note = each N may independently be thymine DNA or uracil RNA
  misc_feature 13: note = each N may independently be thymine DNA or uracil RNA
  misc_feature 16: note = each N may independently be thymine DNA or uracil RNA
nngagagcgangngcnggac

SEQ ID NO: 49 - DNA, 20 bp, synthetic construct
  misc_feature 1..2: note = RNA
  source 1..20: mol_type = other DNA
  misc_feature 16: note = RNA
ttgagagcgatgtgctggac

SEQ ID NO: 50 - DNA, 20 bp, synthetic construct
  misc_feature 1..2: note = RNA
  source 1..20: mol_type = other DNA
  misc_feature 16: note = RNA
  misc_feature 1..5: note = 2'-O-methoxyethylribose (MOE) modified nucleotides
  misc_feature 16..20: note = 2'-O-methoxyethylribose (MOE) modified nucleotides
  misc_feature 6..15: note = phosphorothioated DNA backbone
ttgagagcgatgtgctggac

SEQ ID NO: 51 - DNA, 17 bp, synthetic construct
  source 1..17: mol_type = other DNA
  misc_feature 3: note = each N may independently be thymine DNA or uracil RNA
  misc_feature 5: note = each N may independently be thymine DNA or uracil RNA
  misc_feature 8: note = each N may independently be thymine DNA or uracil RNA
  misc_feature 11: note = each N may independently be thymine DNA or uracil RNA
  misc_feature 13: note = each N may independently be thymine DNA or uracil RNA
  misc_feature 15..16: note = each N may independently be thymine DNA or uracil RNA
gcngngangangncnnc

SEQ ID NO: 52 - DNA, 17 bp, synthetic construct
  misc_feature 3: note = RNA
  source 1..17: mol_type = other DNA
  misc_feature 15..16: note = RNA
gctgtgatgatgtcttc

SEQ ID NO: 53 - DNA, 17 bp, synthetic construct
  misc_feature 3: note = RNA
  source 1..17: mol_type = other DNA
  misc_feature 15..16: note = RNA
  misc_feature 1..4: note = 2'-O-methoxyethylribose (MOE) modified nucleotides
  misc_feature 14..17: note = 2'-O-methoxyethylribose (MOE) modified nucleotides
  misc_feature 5..13: note = phosphorothioated DNA backbone
gctgtgatgatgtcttc

SEQ ID NO: 54 - DNA, 17 bp, synthetic construct
  source 1..17: mol_type = other DNA
  misc_feature 2: note = each N may independently be thymine DNA or uracil RNA
  misc_feature 4: note = each N may independently be thymine DNA or uracil RNA
  misc_feature 7: note = each N may independently be thymine DNA or uracil RNA
angngcnggaccccagc

SEQ ID NO: 55 - DNA, 17 bp, synthetic construct
  misc_feature 2: note = RNA
  source 1..17: mol_type = other DNA
  misc_feature 4: note = RNA
atgtgctggaccccagc

SEQ ID NO: 56 - DNA, 17 bp, synthetic construct
  misc_feature 4: note = RNA
  source 1..17: mol_type = other DNA
  misc_feature 2: note = RNA
  misc_feature 1..4: note = 2'-O-methoxyethylribose (MOE) modified nucleotides
  misc_feature 14..17: note = 2'-O-methoxyethylribose (MOE) modified nucleotides
  misc_feature 5..13: note = phosphorothioated DNA backbone
atgtgctggaccccagc

SEQ ID NO: 57 - DNA, 17 bp, synthetic construct
  misc_feature 6..7: note = each N may be independently thymine DNA or uracil RNA
  source 1..17: mol_type = other DNA
  misc_feature 2: note = each N may be independently thymine DNA or uracil RNA
angacnnccaggaagag

SEQ ID NO: 58 - DNA, 17 bp, synthetic construct
  misc_feature 2: note = RNA
  source 1..17: mol_type = other DNA
atgacttccaggaagag

SEQ ID NO: 59 - DNA, 17 bp, synthetic construct
  misc_feature 2: note = RNA
  source 1..17: mol_type = other DNA
  misc_feature 1..4: note = 2'-O-methoxyethylribose (MOE) modified nucleotides
  misc_feature 14..17: note = 2'-O-methoxyethylribose (MOE) modified nucleotides
  misc_feature 5..13: note = phosphorothioated DNA backbone
atgacttccaggaagag

SEQ ID NO: 60 - DNA, 17 bp, synthetic construct
  source 1..17: mol_type = other DNA
  misc_feature 2..5: note = each N may be independently thymine DNA or uracil RNA
  misc_feature 15..16: note = each N may be independently thymine DNA or uracil RNA
annnnggcgccccgnnc

SEQ ID NO: 61 - DNA, 17 bp, synthetic construct
  misc_feature 2..4: note = RNA
  source 1..17: mol_type = other DNA
  misc_feature 15..16: note = RNA
attttggcgccccgttc

SEQ ID NO: 62 - DNA, 17 bp, synthetic construct
  misc_feature 2..4: note = RNA
  source 1..17: mol_type = other DNA
  misc_feature 15..16: note = RNA
  misc_feature 1..4: note = 2'-O-methoxyethylribose (MOE) modified nucleotides
  misc_feature 14..17: note = 2'-O-methoxyethylribose (MOE) modified nucleotides
  misc_feature 5..13: note = phosphorothioated DNA backbone
attttggcgccccgttc

SEQ ID NO: 63 - DNA, 22 bp, synthetic construct
  source 1..22: mol_type = other DNA
ccagatccaggaatatgtggac

SEQ ID NO: 64 - DNA, 20 bp, synthetic construct
  source 1..20: mol_type = other DNA
ctccaggcctctctctctca

SEQ ID NO: 65 - DNA, 19 bp, synthetic construct
  source 1..19: mol_type = other DNA
gcggaagggtacagccaat

SEQ ID NO: 66 - DNA, 21 bp, synthetic construct
  source 1..21: mol_type = other DNA
caggctgtgatacatgtggcg

(19) United States
(12) Patent Application Publication
(10) Pub. No.: US 2024/0344072 A1
(43) Pub. Date: Oct. 17, 2024

(54) TREATMENT FOR TYROSINE DEGRADATION-ASSOCIATED DISORDERS

(71) Applicant: UCL BUSINESS LTD, London (GB)

(72) Inventors: Haiyan Zhou, London (GB); Paul Gissen, London (GB); Bruno Doreste, London (GB)

(73) Assignee: UCL BUSINESS LTD, London (GB)

(21) Appl. No.: 18/575,039

(22) PCT Filed: Jul. 4, 2022
(86) PCT No.: PCT/GB2022/051714

§ 371 Date: Dec. 28, 2023

(30) Foreign Application Priority Data
Jul. 5, 2021 (GB) 2109675.5

(51) Int. Cl. C12N 15/113; A61K 31/122; A61K 31/7125; A61K 31/713; A61P 3/00

(52) U.S. Cl. CPC C12N 15/1137; A61K 31/122; A61K 31/7125; A61K 31/713; A61P 3/00; C12Y 113/11027; C12N 2310/11; C12N 2310/14; C12N 2310/315; C12N 2310/3231; C12N 2310/341; C12N 2310/351; C12N 2310/3525

(57) ABSTRACT

The disclosure relates to a method of treating a disorder of the tyrosine degradation pathway in a subject, and a composition for use in said method. The disclosure also relates to a nucleic acid silencing molecule that reduces the expression of an enzyme involved in the tyrosine degradation pathway.

### FIELD OF THE DISCLOSURE

The disclosure relates to a method of treating a disorder of the tyrosine degradation pathway in a subject, and a composition for use in said method. The disclosure also relates to a nucleic acid silencing molecule that reduces the expression of an enzyme involved in the tyrosine degradation pathway.

### BACKGROUND

Several inherited metabolic disorders affect the tyrosine degradation pathway. These disorders arise when an individual has a mutation in one of the genes which encode the enzymes involved in this pathway. The mutation results in a block in the pathway and causes an accumulation of toxic metabolites upstream of the step catalysed by the affected enzyme.

In more detail, Hereditary Tyrosinemia Type I (HT1) is a disease caused by mutations in the fumarylacetoacetate hydrolase (FAH) gene. HT1 has an overall incidence of 1/100,000 births. There are areas of higher incidence in Quebec, Canada (1/1854 births), Finland (1/5000 births), and an immigrant population from Pakistan in the West Midlands (U.K., 1/2628). Patients typically present with failure to thrive and hepatomegaly. There are three sub-types of HT1 (acute, sub-acute, and chronic) and, left without treatment, death may occur by 10 years of age. Metabolites such as 4-fumarylacetoacetate (FA) and succinylacetone (SA) accumulate upstream of FAH. These metabolites are toxic to the liver and kidneys, and so HT1 results in clinical features that include progressive liver failure, development of hepatocellular carcinoma (HCC), renal tubular dysfunction, and porphyria-like psychiatric symptoms accompanied by neurological crisis and diaphragmatic paralysis. High plasma levels of tyrosine and alpha-fetoprotein (AFP, which is a biomarker of hepatocellular carcinoma) are indicative of HT1, while the most reliable diagnostic marker is the excretion of succinylacetone in the urine.

The only medication available to treat HT1 is nitisinone (NTBC). NTBC is an inhibitor of 4 hydroxyphenylpyruvate dioxygenase (HPD), the second enzyme in the tyrosine degradation pathway. To control HT1, NTBC requires life-long dosing. Due to NTBC's short half-life, patients have to take dozens of tablets several times per day. As a result, patient non-compliance is common. Such non-compliance can lead to a rapid deterioration in health marked by neurologic crisis (including diaphragmatic paralysis) and an increased risk of hepatocellular carcinoma. Patients on NTBC require tyrosine restricted diet because 4 hydroxyphenylpyruvate dioxygenase inhibition leads to high levels of tyrosine without accumulation of toxic compounds: fumarylacetoacetate (FAA), maleylacetoacetate (MAA) and succinyl acetone (SA).

Similarly, Alkaptonuria (AKU) is a disease cause by the absence of the 3rd enzyme of the tyrosine-degradation pathway, homogentisate 1,2-dioxygenase (HGD). AKU has an incidence of 1/250,000-1,000,000. Defects in HGD lead to the accumulation of homogentisic acid (HA), which accumulates and turns into a pigment deposited in tissues. By the age of 30 years patients develop arthritis; stones in the gall-bladder, prostate, and salivary glands; rupture of tendons, ligaments and muscles; bone fractures; and failure of cardiac valves. NTBC can be administered to inhibit HPD and reduce the production of HA, but issues of non-compliance again apply.

Previously, as an alternative to NTBC, attempts have been made to treat HT1 with gene replacement therapies using viral vectors to introduce FAH expression in hepatocytes. These have been trialled in mice using a wide variety of viral delivery mechanisms. For instance, Overturf et al. (Nat Genet. 1996; 12(3):266-273) attempted this first using a retroviral vector, and then an adenoviral vectors (Overturf et al., Hum Gene Ther. 1997; 8(5):513-521). Paulk et al. (Hepatology. 2010; 51(4):1200-1208) attempted the delivery of a functional FAH using adeno-associated virus. A self-inactivating lentiviral delivery system was also attempted by Rittelmeyer et al. (Hepatology. 2013; 58(1):397-408), and an AAV CRISPR/Cas9 system by Yin et al. (Nat Biotechnol. 2014; 32(6):551-553). All of these attempts corrected some hepatocytes in vivo, but not all. This results in “mosaicism” in the liver, which becomes significant as the carcinogenic effects of FAA and MAA are autologous. As viral delivery vectors are immunogenic and can only be administered once, it is not possible to re-administer the gene replacement therapy in an attempt to homogenise the liver. Consequently, if not all hepatocytes are corrected the first time, the remaining uncorrected cells will lead to the development of neoplastic nodules and hepatocellular carcinomas. For example, Overturf et al. (1997) observed over 77% of treated mice developed neoplastic nodules in the liver within 6-9 months of treatment with adenoviral vectors carrying FAH18.

There is therefore a need for improved treatments for disorders of the tyrosine degradation pathway.

### SUMMARY OF THE DISCLOSURE

The present inventors have demonstrated that it is possible to reduce gene expression of 4-hydroxyphenylpyruvate dioxygenase (HPD) using a nucleic acid silencing molecule. As HPD is an upstream (and, therefore, an early) enzyme in the tyrosine degradation pathway, reducing HPD expression may reduce the accumulation of toxic metabolites arising from metabolic disorders affecting the pathway downstream. In this way, symptoms of the disorders may be alleviated.

Use of a nucleic acid silencing molecule that reduces expression of HPD to treat disorders of the tyrosine degradation pathway is associated with several advantages. Firstly, HPD is an early enzyme in the pathway (see FIG. 1), and so all downstream disorders may be treated by reducing expression of HPD. For example, a nucleic acid silencing molecule that reduces expression HPD may be used to treat a disorder arising from one or more mutations in HGD, such as AKU. A nucleic acid silencing molecule that reduces expression of HPD may be used to treat a disorder arising from one or more mutations in FAH, such as HT1.

Secondly, disorders of the tyrosine degradation pathway are typically treated with NTBC. Due to NTBC's short half-life, patients have to take dozens of tablets several times per day for life and therefore patient non-compliance is common. This is undesirable, because non-compliance can lead to a rapid deterioration in health and be lethal. Nucleic acid silencing molecules have a long half-life, which would allow treatment once every few weeks or months. In this way, non-compliance issues can be overcome, improving the health outcomes and quality of life of patients.

Thirdly, nucleic acid silencing molecules are non-immunogenic. They can be repeatedly administered to ensure that all hepatocytes are treated. This avoids mosaicism in the liver, and prevents uncorrected hepatocytes from developing into neoplastic nodules and hepatocellular carcinomas. In this way, the nucleic acid silencing molecules are advantageous over the gene replacement therapies proposed previously.

Fourthly, nucleic acid silencing molecules can easily be targeted to the organs and tissues typically affected by HT1, for instance by GalNAc conjugation or packaging in lipid nanoparticles. This would ensure that HPD is suppressed to a sufficient extent to eliminate the accumulation of toxic metabolites systemically. Furthermore, HPD can be suppressed within every hepatocyte, preventing the formation of a mosaic liver that would lead to the development of HCC long-term.

In summary, use of a nucleic acid silencing molecule that reduces expression of HPD may prevent the accumulation of toxic metabolites resulting from disorders of the tyrosine degradation pathway. Clinical manifestations of disease are therefore alleviated.

The nucleic acid silencing molecule is suited to good patient compliance, and so health outcomes are improved. The nucleic acid silencing molecule can be targeted to key tissues, such as the liver, and may be administered repeatedly to avoid “mosaicing” that can lead to detrimental outcomes.

The disclosure therefore provides a method of treating a disorder of the tyrosine degradation pathway in a subject, comprising administering to the subject a composition comprising a nucleic acid silencing molecule that reduces the expression of 4-hydroxyphenylpyruvate dioxygenase (HPD). The disclosure also provides:
a composition for use in a method of treating a disorder of the tyrosine degradation pathway in a subject, wherein the composition comprises a nucleic acid silencing molecule that reduces the expression of 4-hydroxyphenylpyruvate dioxygenase (HPD), and the method comprises administering the nucleic acid silencing molecule to the subject;
a nucleic acid silencing molecule that reduces the expression of 4-hydroxyphenylpyruvate dioxygenase (HPD); and
an in vitro method for reducing expression of HPD in a cell, comprising contacting the cell with the nucleic acid silencing molecule of the disclosure.

### BRIEF DESCRIPTION OF THE FIGURES

FIG. 1: Tyrosine degradation pathway) and the proposed nucleic acid silencing approach as an alternative therapy to NTBC in HT1. HT1: Tyrosinemia type I; AKU: Alkaptonuria; FAH: fumarylacetoacetate hydrolase; FAA: 4-fumarylacetoacetate; MAA: maleylacetoacetate; SA: succinylacetone. In HT1 the last enzyme of the pathway (FAH) is dysfunctional. This leads to the accumulation of toxic metabolites FAA, MAA and SA. MAA and FAA stay intracellular and are carcinogenic. SA is secreted and is a good biomarker and possibly has an effect in other organs such as pancreas, kidney, heart. In AKU the enzyme HGD is absent, leading to accumulations of homogentisic acid. The current standard of care relies on NTBC which directly inhibits HPD activity, and therefore prevents the formation of downstream toxic metabolites in both AKU and HT1. The alternative approach described herein applies a gene silencing approach, and may conjugate a nucleic acid silencing molecule (e.g. AON or siRNA) with GalNAc to allow hepatocyte-specific delivery. GalNAc mediates endocytic uptake by binding to the ASPGR receptor. In the endosome, the GalNAc-AON conjugate is cleaved, the ASPGR receptor is recycled, and the AON is released. The AON then binds to HPD mRNA and triggers RNase H mediated HPD mRNA degradation. The downregulation of HPD expression then inhibits the pathway and prevents the accumulation of FA, MAA and secretion of SA in HT1 or HA in AKU.

FIG. 2: Designed AONs downregulate HPD expression in the human hepatic cell line HepG2. A) Screening of AONs, with AONs 1, 7, and 11 showing strong silencing effect (tested at 50 nM); B) Dose response curve of AON 1 in HepG2 cells at 100 nM. IC50=9.504 nM; C) Dose response curve of AON7 in HepG2 cells. IC50=11.43 nM; D) Western blot illustrating the effect of AON7 on downregulating HPD expression (HPD: red, GAPDH: Green). E) Relative Quantification of HPD protein (normalised to GAPDH) showing the dose-dependent response to AON7. ****p<0.0001 ***p<0.001, **p<0.01, *p<0.05.

FIG. 3: Screening of siRNAs against HPD. A-C) Fold change in expression of HPD after siRNAs 1-3 treatment. D-F) Dose response curve of siRNA 1 (IC₅₀=15.57 μM), siRNA 2 (IC₅₀=10.78 μM) and siRNA 3 (IC₅₀=8.606 μM). G) Western blot showing dramatic downregulation of HPD (red) in HepG2s treated with l0 nM of each candidate siRNA compared to ß-tubulin (green). H) Relative quantification of HPD protein downregulation in samples treated with l0 nM of each siRNA candidate or scramble control, normalised to ß-tubulin. (****p<0.0001).

FIG. 4: Effect of AON-7 on Hpd expression in liver tissues from NTBC-treated Fah knockout (Fah^{-/-}) mice. Mice received subcutaneous injection of AON (MOE) at 50 μg/g from postnatal day 0 (PO) at one-week interval for 5 weeks. One week after the last injection, liver tissues were collected for the measurement of Hpd. MOE AON downregulated: A) Hpd mRNA measured by quantitative RT-PCR, and B) Hpd protein measured by western blotting. **p<0.01.

FIG. 5: Effect of GalNAc conjugated AON-7 (MOE) on Hpd mRNA expression in NTBC-treated Fah^{-/-} mice. 3-week-old Fah^{-/-} mice pre-treated with NTBC were injected with a single dose of GalNAc-AON at 5 μg/g subcutaneously. Hpd mRNA expression in liver was reduced at one week after the injection.

FIG. 6: A single subcutaneous injection of GalNAc-AON7b at 5 μg/g in 3-week-old Fah^{-/-} mice pre-treated with NTBC, significantly increased bodyweight gains in one week, when compared to the No-AON littermate controls. *p<0.05.

FIG. 7: GalNAc-AON7b treatment increased the survival of Fah^{-/-} mice. Fah^{-/-} mice were maintained under NTBC water. New-born Fah^{-/-} mice were given a single dose of GalNAc-AON at 5 μg/g subcutaneously at 2 weeks interval. NTBC water was withdrawn from both GalNAc-AON treated group and No-AON control group at 4 weeks old. Survival curves were analysed by Log-rank test. The mean survival (on days after NTBC was withdrawn): 15 days in GalNAc-AON group (n=7); 5.5 days in No-AON control group (n=4). P=0.0028

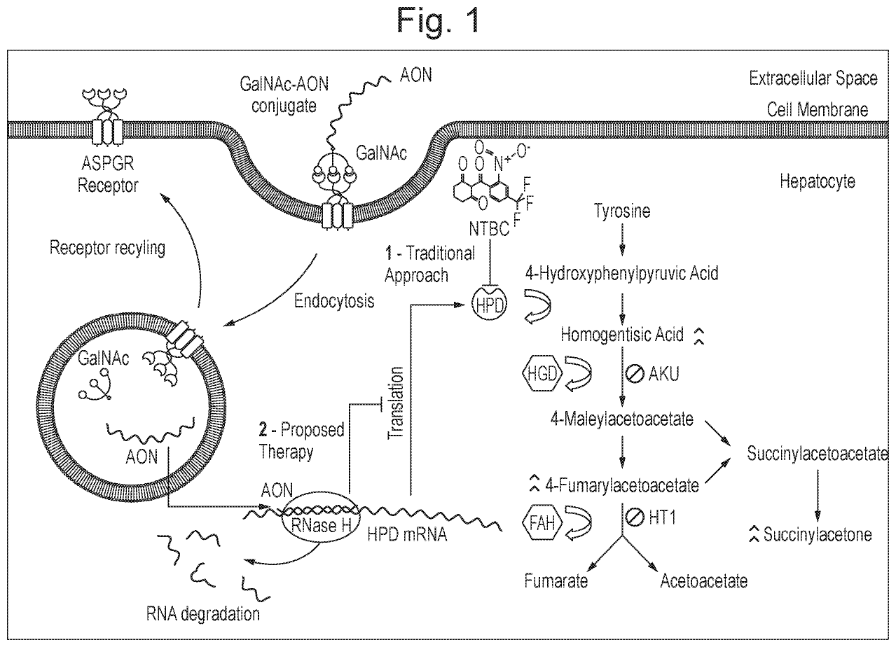

### BRIEF DESCRIPTION OF THE SEQUENCE LISTING

SEQ ID NO: 1—nucleic acid silencing molecule of the invention. Each N may be independently selected from T and U.

SEQ ID NO: 2—nucleic acid sequence of a preferred nucleic acid silencing molecule sequence according to SEQ ID NO: 1.

SEQ ID NO: 3—nucleic acid sequence of AON1, a LNA+PS-modified sequence of SEQ ID NO: 2. * indicates phosphorothioated DNA backbone; {n} is LNA.

SEQ ID NO: 4—nucleic acid silencing molecule of the invention. Each N may be independently selected from T and U.

SEQ ID NO: 5—nucleic acid sequence of a preferred nucleic acid silencing molecule sequence according to SEQ ID NO: 4.

SEQ ID NO: 6—nucleic acid sequence of AON2, a LNA+PS-modified sequence of SEQ ID NO: 5. * indicates phosphorothioated DNA backbone; {n} is LNA.

SEQ ID NO: 7—nucleic acid silencing molecule of the invention. Each N may be independently selected from T and U.

SEQ ID NO: 8—nucleic acid sequence of a preferred nucleic acid silencing molecule sequence according to SEQ ID NO: 7.

SEQ ID NO: 9—nucleic acid sequence of AON3, a LNA+PS-modified sequence of SEQ ID NO: 8. * indicates phosphorothioated DNA backbone; {n} is LNA.

SEQ ID NO: 10—nucleic acid silencing molecule of the invention. Each N may be independently selected from T and U.

SEQ ID NO: 11—nucleic acid sequence of a preferred nucleic acid silencing molecule sequence according to SEQ ID NO: 10.

SEQ ID NO: 12—nucleic acid sequence of AON4, a 2′-O-Me+PS-modified sequence of SEQ ID NO: 11. * indicates phosphorothioated DNA backbone; <n> is 2′-O-Me.

SEQ ID NO: 13—nucleic acid sequence of AON10, a LN+PS-modified sequence of SEQ ID NO: 11. * indicates phosphorothioated DNA backbone; {n} is LNA.

SEQ ID NO: 14—nucleic acid silencing molecule of the invention. Each N may be independently selected from T and U.

SEQ ID NO: 15—nucleic acid sequence of a preferred nucleic acid silencing molecule sequence according to SEQ ID NO: 14.

SEQ ID NO: 16—nucleic acid sequence of AON5, a 2′-O-Me+PS-modified sequence of SEQ ID NO: 15. * indicates phosphorothioated DNA backbone; <n> is 2′-O-Me.

SEQ ID NO: 17—nucleic acid silencing molecule of the invention. Each N may be independently selected from T and U.

SEQ ID NO: 18—nucleic acid sequence of a preferred nucleic acid silencing molecule sequence according to SEQ ID NO: 17.

SEQ ID NO: 19—nucleic acid sequence of AON6, a 2′-O-Me+PS-modified sequence of SEQ ID NO: 18. * indicates phosphorothioated DNA backbone; <n> is 2′-O-Me.

SEQ ID NO: 20—nucleic acid silencing molecule of the invention. Each N may be independently selected from T and U.

SEQ ID NO: 21—nucleic acid sequence of a preferred nucleic acid silencing molecule sequence according to SEQ ID NO: 20.

SEQ ID NO: 22—nucleic acid sequence of AON7a, a 2′-O-Me+PS-modified sequence of SEQ ID NO: 21. * indicates phosphorothioated DNA backbone; <n> is 2′-O-Me.

SEQ ID NO: 23—nucleic acid sequence of AON7b, a MOE+PS-modified sequence of SEQ ID NO: 21. * indicates phosphorothioated DNA backbone; [n] is MOE.

SEQ ID NO: 24—nucleic acid silencing molecule of the invention. Each N may be independently selected from T and U.

SEQ ID NO: 25—nucleic acid sequence of a preferred nucleic acid silencing molecule sequence according to SEQ ID NO: 24.

SEQ ID NO: 26—nucleic acid sequence of AON8, a 2′-O-Me+PS-modified sequence of SEQ ID NO: 25. * indicates phosphorothioated DNA backbone; <n> is 2′-O-Me.

SEQ ID NO: 27—nucleic acid silencing molecule of the invention. Each N may be independently selected from T and U.

SEQ ID NO: 28—nucleic acid sequence of a preferred nucleic acid silencing molecule sequence according to SEQ ID NO: 27.

SEQ ID NO: 29—nucleic acid sequence of AON9, a LNA+PS-modified sequence of SEQ ID NO:28. * indicates phosphorothioated DNA backbone; {n} is LNA.

SEQ ID NO: 30—nucleic acid silencing molecule of the invention. Each N may be independently selected from T and U.

SEQ ID NO: 31—nucleic acid sequence of a preferred nucleic acid silencing molecule sequence according to SEQ ID NO: 30.

SEQ ID NO: 32—nucleic acid sequence of a AON11, a LNA+PS-modified sequence of SEQ ID NO: 31. * indicates phosphorothioated DNA backbone; {n} is LNA.

SEQ ID NO: 33—nucleic acid silencing molecule of the invention. Each N may be independently selected from T and U.

SEQ ID NO: 34—nucleic acid sequence of a preferred nucleic acid silencing molecule sequence according to SEQ ID NO: 33.

SEQ ID NO: 35—nucleic acid sequence of AON12, a LNA+PS-modified sequence of SEQ ID NO: 34. * indicates phosphorothioated DNA backbone; {n} is LNA.

SEQ ID NO: 36—nucleic acid silencing molecule of the invention. Each N may be independently selected from dT,T and U.

SEQ ID NO: 37—nucleic acid sequence of siRNAl, a nucleic acid silencing molecule sequence according to SEQ ID NO: 36.

SEQ ID NO: 38—nucleic acid silencing molecule of the invention. Each N may be independently selected from dT, T and U.

SEQ ID NO: 39—nucleic acid sequence of siRNA2, a nucleic acid silencing molecule sequence according to SEQ ID NO: 38.

SEQ ID NO: 40—nucleic acid silencing molecule of the invention. Each N may be independently selected from dT, T and U.

SEQ ID NO: 41—nucleic acid sequence of siRNA3, a nucleic acid silencing molecule sequence according to SEQ ID NO: 40.

SEQ ID NO: 42—nucleic acid sequence of the human HPD gene.

SEQ ID NO: 43—nucleic acid sequence of mRNA encoded by the human HPD gene.

SEQ ID NO: 44—amino acid sequence of human HPD protein.

SEQ ID NO: 45—nucleic acid silencing molecule of the invention. Each N may be independently selected from T and U.

SEQ ID NO: 46—nucleic acid sequence of a preferred nucleic acid silencing molecule sequence according to SEQ ID NO: 45.

SEQ ID NO: 47—nucleic acid sequence of AON13, a MOE+PS-modified sequence of SEQ ID NO: 46. * indicates phosphorothioated DNA backbone; [n] is MOE.

SEQ ID NO: 48—nucleic acid silencing molecule of the invention. Each N may be independently selected from T and U.

SEQ ID NO: 49—nucleic acid sequence of a preferred nucleic acid silencing molecule sequence according to SEQ ID NO: 48.

SEQ ID NO: 50—nucleic acid sequence of AON14, a MOE+PS-modified sequence of SEQ ID NO: 49. * indicates phosphorothioated DNA backbone; [n] is MOE.

SEQ ID NO: 51—nucleic acid silencing molecule of the invention. Each N may be independently selected from T and U.

SEQ ID NO: 52—nucleic acid sequence of a preferred nucleic acid silencing molecule sequence according to SEQ ID NO: 51.

SEQ ID NO: 53—nucleic acid sequence of AON15, a MOE+PS-modified sequence of SEQ ID NO: 52. * indicates phosphorothioated DNA backbone; [n] is MOE.

SEQ ID NO: 54—nucleic acid silencing molecule of the invention. Each N may be independently selected from T and U.

SEQ ID NO: 55—nucleic acid sequence of a preferred nucleic acid silencing molecule sequence according to SEQ ID NO: 54.

SEQ ID NO: 56—nucleic acid sequence of AON16, a MOE+PS-modified sequence of SEQ ID NO: 55. * indicates phosphorothioated DNA backbone; [n] is MOE.

SEQ ID NO: 57—nucleic acid silencing molecule of the invention. Each N may be independently selected from T and U.

SEQ ID NO: 58—nucleic acid sequence of a preferred nucleic acid silencing molecule sequence according to SEQ ID NO: 57.

SEQ ID NO: 59—nucleic acid sequence of AON17, a MOE+PS-modified sequence of SEQ ID NO: 58. * indicates phosphorothioated DNA backbone; [n] is MOE.

SEQ ID NO: 60—nucleic acid silencing molecule of the invention. Each N may be independently selected from T and U.

SEQ ID NO: 61—nucleic acid sequence of a preferred nucleic acid silencing molecule sequence according to SEQ ID NO: 60.

SEQ ID NO: 62—nucleic acid sequence of AON18, a MOE+PS-modified sequence of SEQ ID NO: 61. * indicates phosphorothioated DNA backbone; [n] is MOE.

SEQ ID NO: 63—HPD forward primer used in the Example.

SEQ ID NO: 64—HPD reverse primer used in the Example.

SEQ ID NO: 65—RPL19 forward primer used in the Example.

SEQ ID NO: 66—RPL19 reverse primer used in the Example.

### DETAILED DESCRIPTION

It is to be understood that different applications of the disclosed methods and products may be tailored to the specific needs in the art. It is also to be understood that the terminology used herein is for the purpose of describing particular embodiments of the disclosure only, and is not intended to be limiting.

All publications, patents and patent applications cited herein, whether supra or infra, are hereby incorporated by reference in their entirety.

### General Definitions

Unless defined otherwise, technical and scientific terms used herein have the same meaning as commonly understood by a person skilled in the art to which this disclosure belongs.

As used in this specification and the appended claims, the singular forms “a”, “an”, and “the” include plural referents unless the content clearly dictates otherwise. Thus, for example, reference to “a cell” includes “cells”, reference to “an antisense oligonucleotide” includes two or more such antisense oligonucleotides, and the like.

In general, the term “comprising” is intended to mean including but not limited to. For example, the phrase “a nucleic acid silencing molecule comprising RNA” should be interpreted to mean that the nucleic acid silencing molecule contains RNA, but that the nucleic acid silencing molecule may contain additional nucleic acids.

In some aspects of the disclosure, the word “comprising” is replaced with the phrase “consisting of”. The term “consisting of” is intended to be limiting. For example, the phrase “a nucleic acid silencing molecule consisting of RNA” should be understood to mean that the nucleic acid silencing molecule contains RNA and no additional nucleic acids.

The terms “protein” and “polypeptide” are used interchangeably herein, and are intended to refer to a polymeric chain of amino acids of any length.

For the purpose of this disclosure, in order to determine the percent identity of two sequences (such as two polynucleotide or two polypeptide sequences), the sequences are aligned for optimal comparison purposes (e.g., gaps can be introduced in a first sequence for optimal alignment with a second sequence). The nucleotide residues at nucleotide positions are then compared. When a position in the first sequence is occupied by the same nucleotide residue as the corresponding position in the second sequence, then the nucleotides are identical at that position. The percent identity between the two sequences is a function of the number of identical positions shared by the sequences (i.e., % identity=number of identical positions/total number of positions in the reference sequence×100).

Typically the sequence comparison is carried out over the length of the reference sequence. For example, if the user wished to determine whether a given (“test”) sequence has a certain percentage identity to SEQ ID NO: X, SEQ ID NO: X would be the reference sequence. For example, to assess whether a sequence is at least 80% identical to SEQ ID NO: X (an example of a reference sequence), the skilled person would carry out an alignment over the length of SEQ ID NO: X, and identify how many positions in the test sequence were identical to those of SEQ ID NO: X. If at least 80% of the positions are identical, the test sequence is at least 80% identical to SEQ ID NO: X. If the sequence is shorter than SEQ ID NO: X, the gaps or missing positions should be considered to be non-identical positions.

The skilled person is aware of different computer programs that are available to determine the homology or identity between two sequences. For instance, a comparison of sequences and determination of percent identity between two sequences can be accomplished using a mathematical algorithm

### Nucleic Acid Silencing Molecule

Disclosed herein is a nucleic acid silencing molecule that reduces the expression of 4-hydroxyphenylpyruvate dioxygenase (HPD).

Such a silencing molecule may be used therapeutically to reduce the expression of HPD. By reducing the expression of HPD, the tyrosine degradation pathway may be blocked or inhibited at its early steps, because HPD is the second enzyme in the pathway. By blocking or inhibiting the top of the pathway in this way, the formation of downstream metabolites may be prevented or reduced. Accumulation of toxic metabolites, such as hepatotoxic or nephrotoxic metabolites, may thus be prevented or reduced. By preventing or reducing the accumulation of toxic metabolites, disorders of the tyrosine degradation pathway may be treated. For example, clinical signs or symptoms of such disorders may be reduced or abolished. Clinical signs or symptoms may be stopped from progressing. Treatment of disorders of the tyrosine degradation pathway using the nucleic acid silencing molecule disclosed herein may have advantages over other treatment methods, such as administration of NTBC, as set out above.

Other uses of the nucleic acid silencing molecule disclosed herein may also be envisaged. For example, the nucleic acid silencing molecule may be used to experimentally reduce the expression of HPD in vivo or in vitro. In other words, the nucleic acid silencing molecule may be used as a research tool, for instance to investigate the tyrosine degradation pathway.

### Reducing the Expression of HPD

The nucleic acid silencing molecule disclosed herein reduces the expression of 4-hydroxyphenylpyruvate dioxygenase (HPD). The nucleic acid silencing molecule may, for example, reduce the expression of HPD mRNA. The nucleic acid silencing molecule may, for example, reduce the expression of HPD protein. The nucleic acid silencing molecule may, for example, reduce the expression of HPD mRNA and HPD protein.

The nucleic acid silencing molecule may, for example, reduce the expression of HPD in a cell. The cell may, for example, be human or from a human cell line. The cell may be a liver cell, or from a liver cell line. The cell may, for example, be in vivo, ex vivo or in vitro. The cell may be a cell contacted with the nucleic acid silencing molecule. Reduced expression of HPD in a cell may, for example, refer to a reduction in the amount of HPD mRNA and/or HPD protein comprised in the cell. Accordingly, a cell that has reduced expression of HPD may, for example, comprise a reduced amount of HPD mRNA. A cell that has reduced expression of HPD may, for example, comprise a reduced amount of HPD protein. A cell that has reduced expression of HPD may, for example, comprise a reduced amount of HPD mRNA and a reduced amount of HPD protein.

The nucleic acid silencing molecule may, for example, reduce the expression of HPD in a cell contacted with the nucleic acid silencing molecule relative to a cell not contacted with the nucleic acid silencing molecule. The cell contacted with the nucleic acid silencing molecule may, for example, be human or from a human cell line. The cell contacted with the nucleic acid silencing molecule may, for example, be in vivo, ex vivo or in vitro. The cell not contacted with the nucleic acid silencing molecule may, for example, be human or from a human cell line. The cell not contacted with the nucleic acid silencing molecule may, for example, be in vivo, ex vivo or in vitro. Preferably, the cell contacted with the nucleic acid silencing molecule and the cell not contacted with the nucleic acid silencing molecule are the same type of cell (e.g. a human cell or a cell from a human cell line). Preferably, the cell contacted with the nucleic acid silencing molecule and the cell not contacted with the nucleic acid silencing molecule are under the same conditions (e.g. in vivo, ex vivo or in vitro). The cell not contacted with the nucleic acid silencing molecule may instead be contacted with a control molecule, such as a mock nucleic acid silencing molecule. Mock nucleic acid silencing molecules (such as mock antisense oligonucleotides) and methods for their design and production are well-known in the art. Reduced expression of HPD in a cell contacted with the nucleic acid silencing molecule may, for example, refer to a reduction in the amount of HPD mRNA and/or HPD protein comprised in the cell relative to a cell not contacted with the nucleic acid silencing molecule. Accordingly, a cell that is contacted with the nucleic acid silencing molecule and that has reduced expression of HPD may comprise a reduced amount of HPD mRNA relative to a cell not contacted with the nucleic acid silencing molecule. A cell that is contacted with the nucleic acid silencing molecule and that has reduced expression of HPD may comprise a reduced amount of HPD protein relative to a cell not contacted with the nucleic acid silencing molecule. A cell that is contacted with the nucleic acid silencing molecule and that has reduced expression of HPD may comprise a reduced amount of HPD mRNA and a reduced amount of HPD protein relative to a cell not contacted with the nucleic acid silencing molecule.

In any of the aspects described above, the nucleic acid silencing molecule may reduce the expression of HPD by at least 30%. In other words, the nucleic acid silencing molecule may reduce the amount of HPD mRNA and/or the amount of HPD protein by at least 30%. The nucleic acid silencing molecule may, for example, reduce the expression of HPD by at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%. In other words, the nucleic acid silencing molecule may, for example, reduce the amount of HPD mRNA and/or the amount of HPD protein by at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%. The nucleic acid silencing molecule may, for example, reduce the expression of HPD by 30% to 99%, such as 35% to 95%, 40% to 90%, 45% to 85%, 50% to 80%, 55% to 75%, or 60% to 70%. That is, the nucleic acid silencing molecule may, for example, reduce amount of HPD mRNA and/or the amount of HPD protein by 30% to 99%, such as 35% to 95%, 40% to 90%, 45% to 85%, 50% to 80%, 55% to 75%, or 60% to 70%.

Preferably, the nucleic acid silencing molecule reduces the expression of HPD in a cell by at least 30%. In other words, the nucleic acid silencing molecule may reduce the amount of HPD mRNA and/or the amount of HPD protein in a cell by at least 30%. Cell types and conditions for assessment of HPD expression are described above. The nucleic acid silencing molecule may, for example, reduce the expression of HPD in a cell by at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%. In other words, the nucleic acid silencing molecule may, for example, reduce the amount of HPD mRNA and/or the amount of HPD protein in a cell by at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%. The nucleic acid silencing molecule may, for example, reduce the expression of HPD in a cell by 30% to 99%, such as 35% to 95%, 40% to 90%, 45% to 85%, 50% to 80%, 55% to 75%, or 60% to 70%. That is, the nucleic acid silencing molecule may, for example, reduce amount of HPD mRNA and/or the amount of HPD protein in a cell by 30% to 99%, such as 35% to 95%, 40% to 90%, 45% to 85%, 50% to 80%, 55% to 75%, or 60% to 70%.

More preferably, the nucleic acid silencing molecule reduces the expression of HPD in a cell contacted with the nucleic acid silencing molecule by at least 30% relative to a cell not contacted with the nucleic acid silencing molecule. In other words, the nucleic acid silencing molecule may reduce the amount of HPD mRNA and/or the amount of HPD protein in a cell contacted with the nucleic acid silencing molecule by at least 30% relative to a cell not contacted with the nucleic acid silencing molecule. Cell types and conditions for assessment of HPD expression are described above. The nucleic acid silencing molecule may, for example, reduce the expression of HPD in a cell contacted with the nucleic acid silencing molecule by at least at least 30%, at least 35%, at least 40%, 45%, at least 50%, at least 55%, at least 60%, at least 65%, at least 70%, at least 75%, at least 80%, at least 85%, at least 90%, at least 95%, at least 98%, at least 99% relative to a cell not contacted with the nucleic acid silencing molecule. In other words, the nucleic acid silencing molecule may, for example, reduce the amount of HPD mRNA and/or the amount of HPD protein in a cell contacted with the nucleic acid silencing molecule by at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99% relative to a cell not contacted with the nucleic acid silencing molecule. The nucleic acid silencing molecule may, for example, reduce the expression of HPD in a cell contacted with the nucleic acid silencing molecule by 30% to 99%, such as 35% to 95%, 40% to 90%, 45% to 85%, 50% to 80%, 55% to 75%, or 60% to 70% relative to a cell not contacted with the nucleic acid silencing molecule. That is, the nucleic acid silencing molecule may, for example, reduce amount of HPD mRNA and/or the amount of HPD protein in a cell contacted with the nucleic acid silencing molecule by 30% to 99%, such as 35% to 95%, 40% to 90%, 45% to 85%, 50% to 80%, 55% to 75%, or 60% to 70% relative to a cell not contacted with the nucleic acid silencing molecule.

In any of the aspects described above, the nucleic acid silencing molecule may reduce the expression of HPD by 100%. In other words, the nucleic acid silencing molecule may completely eliminate the expression of HPD. The nucleic acid silencing molecule may, for example, completely eliminate the expression of HPD mRNA (i.e. reduce the expression of HPD mRNA by 100%). The nucleic acid silencing molecule may, for example, completely eliminate the expression of HPD protein (i.e. reduce the expression of HPD protein by 100%). The nucleic acid silencing molecule may, for example, completely eliminate the expression of HPD mRNA and HPD protein (i.e. reduce the expression of HPD mRNA and HPD protein by 100%).

The nucleic acid silencing molecule may, for example, completely eliminate the expression of HPD in a cell. Accordingly, the cell may not express HPD mRNA and/or HPD protein. The cell may, for example, be human or from a human cell line. The cell may be a liver cell, or from a HPD. The cell may, for example, be in vivo, ex vivo or in vitro. The cell may be a cell contacted with the nucleic acid silencing molecule.

The nucleic acid silencing molecule may, for example, reduce the expression of HPD in a cell contacted with the nucleic acid silencing molecule by 100% relative to a cell not contacted with the nucleic acid silencing molecule. The cell contacted with the nucleic acid silencing molecule may, for example, be human or from a human cell line. The cell contacted with the nucleic acid silencing molecule may, for example, be in vivo, ex vivo or in vitro. The cell not contacted with the nucleic acid silencing molecule may, for example, be human or from a human cell line. The cell not contacted with the nucleic acid silencing molecule may, for example, be in vivo, ex vivo or in vitro. Preferably, the cell contacted with the nucleic acid silencing molecule and the cell not contacted with the nucleic acid silencing molecule are the same type of cell (e.g. a human cell or a cell from a human cell line; a liver cell or a cell from a HPD; a human liver cell or a cell from a human HPD). Preferably, the cell contacted with the nucleic acid silencing molecule and the cell not contacted with the nucleic acid silencing molecule are under the same conditions (e.g. in vivo, ex vivo or in vitro). The cell not contacted with the nucleic acid silencing molecule may instead be contacted with a control molecule, such as a mock nucleic acid silencing molecule. As set out above, mock nucleic acid silencing molecules (such as mock antisense oligonucleotides) and methods for their design and production are well-known in the art.

### Nucleic Acid Silencing Molecule

In the context of the disclosure, a silencing molecule may be defined as a molecule that reduces or eliminates (i.e. knocks down) expression of a target gene. A silencing molecule may, for example, reduce the amount of the mRNA product of target gene. A silencing molecule may, for example, eliminate the mRNA product of target gene. A silencing molecule may, for example, reduce the amount of the protein product of target gene. A silencing molecule may, for example, eliminate the protein product of target gene. In the present disclosure, the target gene is HPD.

In the context of the disclosure, a nucleic acid silencing molecule may be defined as a silencing molecule that comprises or consists of one or more nucleic acids. The nucleic acid silencing molecule may itself be a nucleic acid. The nucleic acid silencing molecule of the disclosure may comprise RNA. The nucleic acid silencing molecule of the disclosure may comprise DNA. The nucleic acid silencing molecule of the disclosure may comprise DNA and RNA. The nucleic acid silencing molecule of the disclosure may consist of RNA. The nucleic acid silencing molecule of the disclosure may consist of DNA. The nucleic acid silencing molecule of the disclosure may consist of DNA and RNA.

The nucleic acid silencing molecule may reduce or eliminate (i.e. knock down) expression of HPD by any mechanism known in the art. The nucleic acid silencing molecule may, for example, bind to a mRNA molecule encoded by the HPD gene to block its translation into protein. The nucleic acid silencing molecule may, for example, bind to a mRNA molecule encoded by the HPD gene to induce degradation (such as enzymatic degradation) of the mRNA. The nucleic acid silencing molecule may, for example, bind to DNA encoding the HPD gene to induce methylation of the DNA and/or its associated histones. The nucleic acid silencing molecule may, for example, bind to DNA encoding HPD to facilitate removal of all or part of the HPD gene by gene editing.

For example, the nucleic acid silencing molecule may comprise or consist of an antisense oligonucleotide (AON). The nucleic acid silencing molecule may comprise or consist of a small interfering RNA (siRNA). The nucleic acid silencing molecule may comprise or consist of a short hairpin RNA (shRNA). The nucleic acid silencing molecule may comprise or consist of a microRNA (miRNA). The nucleic acid silencing molecule may comprise or consist of a CRISPR guide RNA. Preferably, the nucleic acid silencing molecule comprises or consists of an antisense oligonucleotide (AON) or a small interfering RNA (siRNA).

The nucleic acid silencing molecule may be about 10 to about 15000 nucleotides in length, such as about 100 to about 14000, about 200 to about 13000, about 300 to about 12000, about 400 to about 11000, about 400 to about 10000, about 500 to about 9000, about 600 to about 8000, about 700 to about 7000, about 800 to about 6000, about 900 to about 5000, about 1000 to about 4000, or about 2000 to 3000 in length. Preferably, the nucleic acid silencing molecule is less than 100 (such as less than 95, less than 90, less than 85, less than 80, less than 75, less than 70, less than 65, less than 60, less than 55, less than 50, less than 45, less than 40, less than 35, less than 30, less than 25, less than 20, less than 15, or less than 10) nucleotides in length. The nucleic acid silencing molecule may, for example be about 10 to about 50 nucleotides in length. For example, the nucleic acid silencing molecule may be about 10 to about 40, about 10 to about 30, about 10 to about 20, about 20 to about 50, about 20 to about 40, about 20 to about 30, about 30 to about 50, or about 30 to about 40 nucleotides in length. Preferably, the nucleic acid molecule is about 10 to about 30 (such as about 10 to about 20, or about 20 to about 30) nucleotides in length. The nucleic acid molecule may, for example, be about 10, about 11, about 12, about 13, about 14, about 14, about 16, about 17, about 18, about 19, about 20, about 21, about 22, about 23, about 24, about 25, about 26, about 27, about 28, about 29 or about 30 nucleotides in length. The nucleic acid molecule may preferably be about 16 or about 20 nucleic acids in length. Typical lengths of antisense oligonucleotides (AONs), small interfering RNAs (siRNAs), short hairpin RNAs (shRNAs), microRNAs (miRNAs), and CRISPR guide RNAs are well-known in the art.

The nucleic acid silencing molecule may, for example, comprise one or more 2′-O-methoxyethylribose (MOE) modified nucleotides or consist of 2′-O-methoxyethylribose (MOE) modified nucleotides. The nucleic acid silencing molecule may, for example, comprise one or more 2′-O-methyl (2OMe) modified nucleotides or consist of 2′-O-methyl (2OMe) modified nucleotides. The nucleic acid silencing molecule may, for example, comprise one or more locked nucleic acid (LNA) modified nucleotides or consist of locked nucleic acid (LNA) modified nucleotides. The nucleic acid silencing molecule may, for example, comprise one or more nucleotide phosphorothioates or consist of nucleotide phosphorothioates. MOE modified nucleotides, 2OMe modified nucleotides, LNA modified nucleotides and nucleotide phosphorothioates are described in the art.

The nucleic acid silencing molecule may be capable of binding to the HPD gene or to the RNA encoded by the HPD gene. The nucleic acid silencing molecule may be capable of binding to part of the HPD gene or to part of the RNA encoded by the HPD gene. Binding may, for example, be effected by hybridisation.

The nucleic acid silencing molecule may be directed to the nucleic acid sequence of the HPD gene. For example, the nucleic acid silencing molecule may be directed to the DNA of SEQ ID NO: 42. The nucleic acid silencing molecule may be directed to RNA encoded by the HPD gene. For example, the nucleic acid silencing molecule may be directed to mRNA encoded by the HPD gene. The nucleic acid silencing molecule may be directed to RNA encoded by the DNA of SEQ ID NO: 42. For example, the nucleic acid silencing molecule may be directed to mRNA encoded by the DNA of SEQ ID NO: 42. The nucleic acid silencing molecule may be directed to the mRNA of SEQ ID NO: 43. The nucleic acid silencing molecule may be directed to a nucleic acid sequence encoding HPD protein. For example, the nucleic acid silencing molecule may be directed to a nucleic acid sequence encoding the HPD protein of SEQ ID NO: 44. The nucleic acid silencing molecule may be directed to nucleic acid sequence encoding a protein having at least 90% sequence identity to SEQ ID NO: 44. For instance, the nucleic acid silencing molecule may be directed to nucleic acid sequence encoding a protein having at least 91%, at least 92%, at least 93%, at least 94%, at least 95%, at least 96%, at least 97%, at least 98%, or at least 99% sequence identity to SEQ ID NO: 44. A nucleic acid silencing molecule that is “directed to” a particular nucleic acid sequence is capable of binding to (e.g. hybridising to) that nucleic acid sequence.

The nucleic acid silencing molecule may comprise (a) a nucleotide sequence that has at least 75% sequence identity to a nucleotide sequence comprised in a primary transcript of HPD or (b) a nucleotide sequence that is complementary to the nucleotide sequence of (a). The nucleic acid silencing molecule may, for example, comprise or consist of an antisense oligonucleotide (AON). In one aspect, the nucleic acid silencing molecule may comprise a nucleotide sequence that has at least 80%, at least 85%, at least 90%, at least 95%, at least 96%, at least 97%, at least 98%, at least 99%, or 100% sequence identity to a nucleotide sequence comprised in a primary transcript of HPD. In one aspect, the nucleic acid silencing molecule may comprise a nucleotide sequence that has 80% to 100%, 85% to 99%, 90% to 98%, or 95% to 97% sequence identity to a nucleotide sequence comprised in a primary transcript of HPD. In one aspect, the nucleic acid silencing molecule may comprise a nucleotide sequence that is complementary to a nucleotide sequence that has at least 80%, at least 85%, at least 90%, at least 95%, at least 96%, at least 97%, at least 98%, at least 99%, or 100% sequence identity to a nucleotide sequence comprised in a primary transcript of HPD. In one aspect, the nucleic acid silencing molecule may comprise a nucleotide sequence that is complementary to a nucleotide sequence that has 80% to 100%, 85% to 99%, 90% to 98%, or 95% to 97% sequence identity to a nucleotide sequence comprised in a primary transcript of HPD. A primary transcript is the single-stranded ribonucleic acid RNA product synthesised by transcription of DNA, that is processed to yield various mature RNA products such as mRNAs, tRNAs, and rRNAs. The primary transcript may, for example, be a precursor mRNA (pre-mRNA) that is processed to form mRNA. The nucleotide sequence comprised in the primary transcript may comprise one or more of (i) a nucleotide sequence comprised in an exon, (ii) a nucleotide sequence comprised in an intron, (iii) a nucleotide sequence comprised in a 3′ untranslated region, and (iv) a nucleotide sequence comprised in a 5′ untranslated region. The nucleotide sequence comprised in the primary transcript may, for example, comprise: (i); (ii); (iii); (iv); (i) and (ii); (i) and (iii); (i) and (iv); (ii) and (iii); (ii) and (iv); (iii) and (iv); (i), (ii) and (iii); (i), (ii) and (iv); (i), (iii) and (iv); (ii), (iii) and (iv); or (i), (ii), (iii) and (iv).

The nucleic acid silencing molecule may comprise (a) a nucleotide sequence that has at least 75% sequence identity to a nucleotide sequence comprised in a mRNA transcribed from HPD or (b) a nucleotide sequence that is complementary to the nucleotide sequence of (a). The nucleic acid silencing molecule may, for example, comprise or consist of an antisense oligonucleotide (AON). The nucleic acid silencing molecule may, for example, comprise or consist of a small interfering RNA (siRNA). In one aspect, the nucleic acid silencing molecule may comprise a nucleotide sequence that has at least 80%, at least 85%, at least 90%, at least 95%, at least 96%, at least 97%, at least 98%, at least 99%, or 100% sequence identity to a nucleotide sequence comprised in a mRNA transcribed from HPD. In one aspect, the nucleic acid silencing molecule may comprise a nucleotide sequence that has 80% to 100%, 85% to 99%, 90% to 98%, or 95% to 97% sequence identity to a nucleotide sequence comprised in a mRNA transcribed from HPD. In one aspect, the nucleic acid silencing molecule may comprise a nucleotide sequence that is complementary to a nucleotide sequence that has at least 80%, at least 85%, at least 90%, at least 95%, at least 96%, at least 97%, at least 98%, at least 99%, or 100% sequence identity to a nucleotide sequence comprised in a mRNA transcribed from HPD. In one aspect, the nucleic acid silencing molecule may comprise a nucleotide sequence that is complementary to a nucleotide sequence that has 80% to 100%, 85% to 99%, 90% to 98%, or 95% to 97% sequence identity to a nucleotide sequence comprised in a mRNA transcribed from HPD.

The nucleic acid silencing molecule may, for example, target exon 11 of HPD. The present inventors have identified that exon 11 is a target hotspot within HPD. Nucleic acid silencing molecules that target exon 11 may be particular effective in silencing HPD. Accordingly, the nucleic acid silencing molecule may comprise (a) a nucleotide sequence that has at least 75% sequence identity to a nucleotide sequence comprised in exon 11 of a mRNA transcribed from HPD or (b) a nucleotide sequence that is complementary to the nucleotide sequence of (a). The nucleic acid silencing molecule may, for example, comprise or consist of an antisense oligonucleotide (AON). The nucleic acid silencing molecule may, for example, comprise or consist of a small interfering RNA (siRNA). In one aspect, the nucleic acid silencing molecule may comprise a nucleotide sequence that has at least 80%, at least 85%, at least 90%, at least 95%, at least 96%, at least 97%, at least 98%, at least 99%, or 100% sequence identity to a nucleotide sequence comprised in exon 11 of a mRNA transcribed from HPD. In one aspect, the nucleic acid silencing molecule may comprise a nucleotide sequence that has 80% to 100%, 85% to 99%, 90% to 98%, or 95% to 97% sequence identity to a nucleotide sequence comprised in exon 11 of a mRNA transcribed from HPD. In one aspect, the nucleic acid silencing molecule may comprise a nucleotide sequence that is complementary to a nucleotide sequence that has at least 80%, at least 85%, at least 90%, at least 95%, at least 96%, at least 97%, at least 98%, at least 99%, or 100% sequence identity to a nucleotide sequence comprised in exon 11 of a mRNA transcribed from HPD. In one aspect, the nucleic acid silencing molecule may comprise a nucleotide sequence that is complementary to a nucleotide sequence that has 80% to 100%, 85% to 99%, 90% to 98%, or 95% to 97% sequence identity to a nucleotide sequence comprised in exon 11 of a mRNA transcribed from HPD. SEQ ID NOs: 20, 21, 22, 23, 45, 46, 47, 48, 49, 50, 51, 52, 53, 54, 55, 56, 57, 58, 59, 60, 61 and 62 target exon 11 of HPD. AON7b (SEQ ID NO: 23) is considered in the Example. SEQ ID NO: 21 and AON7a (SEQ ID NO: 22) have the same nucleic acid sequence as exemplified AON7b, and SEQ ID NO: 20 is structurally related. An analysis of RNA secondary structure, and a functional domain assay, indicate that SEQ ID NOs: 45, 46, 47, 48, 49, 50, 51, 52, 53, 54, 55, 56, 57, 58, 59, 60, 61 and 62 function in a similar fashion to exemplified AON7b.

The nucleic acid silencing molecule may, for example, comprise or consist of SEQ ID NO: 1, SEQ ID NO: 4, SEQ ID NO: 7, SEQ ID NO: 10, SEQ ID NO: 14, SEQ ID NO: 17, SEQ ID NO: 20, SEQ ID NO: 24, SEQ ID NO: 27, SEQ ID NO: 30, SEQ ID NO: 33, SEQ ID NO: 36, SEQ ID NO: 38, SEQ ID NO: 40, SEQ ID NO: 45, SEQ ID NO: 48, SEQ ID NO 51, SEQ ID NO: 54, SEQ ID NO: 57, or SEQ ID NO: 60. The nucleic acid silencing molecule may, for example, comprise or consist of a nucleotide sequence having at least 75% sequence identity to SEQ ID NO: 1, 4, 7, 10, 14, 17, 20, 24, 27, 30, 33, 36, 38, 40, 45, 48, 51, 54, 57 or 60. For instance, the nucleic acid silencing molecule may comprise or consist of a nucleotide sequence having at least 80%, at least 85%, at least 90%, at least 95%, at least 96%, at least 97%, at least 98% or at least 99% sequence identity to SEQ ID NO: 1, 4, 7, 10, 14, 17, 20, 24, 27, 30, 33, 36, 38, 4045, 48, 51, 54, 57 or 60. Preferably, the nucleic acid silencing molecule comprises or consists of: (a) SEQ ID NO: 2, or a nucleotide sequence having at least 75% sequence identity to SEQ ID NO: 2, (b) SEQ ID NO: 21, or a nucleotide sequence having at least 75% sequence identity to SEQ ID NO: 21; (c) SEQ ID NO: 31, or a nucleotide sequence having at least 75% sequence identity to SEQ ID NO: 31; (d) SEQ ID NO: 45, or a nucleotide sequence having at least 75% sequence identity to SEQ ID NO: 45; (d) SEQ ID NO: 48, or a nucleotide sequence having at least 75% sequence identity to SEQ ID NO: 48; (e) SEQ ID NO: 51, or a nucleotide sequence having at least 75% sequence identity to SEQ ID NO: 51; (f) SEQ ID NO: 54, or a nucleotide sequence having at least 75% sequence identity to SEQ ID NO: 54; (g) SEQ ID NO: 57, or a nucleotide sequence having at least 75% sequence identity to SEQ ID NO: 57; or (h) SEQ ID NO: 60, or a nucleotide sequence having at least 75% sequence identity to SEQ ID NO: 60.

SEQ ID NOs: 1, 4, 7, 10, 14, 17, 20, 24, 27, 30, 33, 45, 48, 51, 54, 57 and 60 contain the nucleic acid “N”. In each instance, N is independently selectable from T (thymine) and U (uracil). For each of SEQ ID NOs: 1, 4, 7, 10, 14, 17, 20, 24, 27, 30, 33, 45, 48, 51, 54, 57 and 60, any number of “N” may be “T”. For each of SEQ ID NOs: 1, 4, 7, 10, 14, 17, 20, 24, 27, 30, 33, 45, 48, 51, 54, 57 and 60 any number of “N” may be “U”. Each of SEQ ID NOs: 1, 4, 7, 10, 14, 17, 20, 24, 27, 30, 33, 45, 48, 51, 54, 57 and 60 may contain any combination of “T” and “U” in the positions designated “N”. Thus, each of SEQ ID NOs: 1, 4, 7, 10, 14, 17, 20, 24, 27, 30, 33, 45, 48, 51, 54, 57 and 60 may be a DNA sequence, a RNA sequence, or a hybrid DNA/RNA sequence.

SEQ ID NOs: 36, 38 and 40 also contain the nucleic acid “N”. In each instance, N is independently selectable from dT (deoxythymine), T (thymine) and U (uracil). For each of SEQ ID NOs: 36, 38 and 40, any number of “N” may be “dT”. For each of SEQ ID NOs: 36, 38 and 40, any number of “N” may be “T”. For each of SEQ ID NOs: 36, 38 and 40, any number of “N” may be “U”. Each of SEQ ID NOs: 36, 38 and 40 may contain any combination of “dT”, “T” and “U” in the positions designated “N”. Thus, each of SEQ ID NOs: 36, 38 and 40 may be a DNA sequence, a RNA sequence, or a hybrid DNA/RNA sequence.

A nucleotide sequence having at least 75% sequence identity to SEQ ID NO: 1, 4, 7, 10, 14, 17, 20, 24, 27, 30, 33, 36, 38, 40, 45, 48, 51, 54, 57 or 60 may comprise one or more nucleotide substitutions with respect to SEQ ID NO: 1, 4, 7, 10, 14, 17, 20, 24, 27, 30, 33, 36, 3,r 40, 45, 48, 51, 54, 57 or 60 respectively. For example, the nucleotide sequence may comprise one, two, three or four substitutions with respect to SEQ ID NO: 1, 4, 7, 30 or 33, which are 16mers. The nucleotide sequence may comprise one, two, three or four substitutions with respect to SEQ ID NO: 45, 51, 54, 57 or 60 which are 17mers. The nucleotide sequence may comprise one, two, three four, or five substitutions with respect to SEQ ID NO: 10, 14, 17, 20, 24, 27 or 48, which are 20mers. The nucleotide sequence may comprise one, two, three four, five, or six substitutions with respect to SEQ ID NO: 36, 38 or 40.

A nucleotide sequence having at least 75% sequence identity to SEQ ID NO: 1, 4, 7, 10, 14, 17, 20, 24, 27, 30, 33, 36, 38, 40, 45, 48, 51, 54, 57 or 60 may comprise one or more nucleotide deletions with respect to SEQ ID NO: 1, 4, 7, 10, 14, 17, 20, 24, 27, 30, 33, 36, 38, 40, 45, 48, 51, 54, 57 or 60 respectively. For example, the nucleotide sequence may comprise one, two, three or four deletions with respect to SEQ ID NO: 1, 4, 7, 30 or 33, which are 16mers. The nucleotide sequence may comprise one, two, three or four deletions with respect to SEQ ID NO: 45, 51, 54, 57 or 60 which are 17mers. The nucleotide sequence may comprise one, two, three, four, or five deletions with respect to SEQ ID NO: 10, 14, 17, 20, 24, 27 or 48, which are 20mers. The nucleotide sequence may comprise one, two, three four, five or six deletions with respect to SEQ ID NO: 36, 38 or 40.

A nucleic acid silencing molecule that comprises or consists of SEQ ID NO: 1 or a nucleotide sequence having at least 75% sequence identity thereto may, for example, comprise or consist of SEQ ID NO: 2 or a nucleotide sequence having at least 75% sequence identity thereto, or SEQ ID NO: 3 (AON1, a LNA+PS-modified sequence of SEQ ID NO: 2) or a nucleotide sequence having at least 75% sequence identity thereto. Accordingly, the nucleic acid silencing molecule may comprise or consist of SEQ ID NO: 2 or a nucleotide sequence having at least 75% sequence identity thereto, or SEQ ID NO: 3 (AON1, a LNA+PS-modified sequence of SEQ ID NO: 2) or a nucleotide sequence having at least 75% sequence identity thereto.

A nucleic acid silencing molecule that comprises or consists of SEQ ID NO: 4 or a nucleotide sequence having at least 75% sequence identity thereto may, for example, comprise or consist of SEQ ID NO: 5 or a nucleotide sequence having at least 75% sequence identity thereto, or SEQ ID NO: 6 (AON2, a LNA+PS-modified sequence of SEQ ID NO: 5) or a nucleotide sequence having at least 75% sequence identity thereto. Accordingly, the nucleic acid silencing molecule may comprise or consist of SEQ ID NO: 5 or a nucleotide sequence having at least 75% sequence identity thereto, or SEQ ID NO: 6 (AON2, a LNA+PS-modified sequence of SEQ ID NO: 5) or a nucleotide sequence having at least 75% sequence identity thereto.

A nucleic acid silencing molecule that comprises or consists of SEQ ID NO: 7 or a nucleotide sequence having at least 75% sequence identity thereto may, for example, comprise or consist of SEQ ID NO: 8 or a nucleotide sequence having at least 75% sequence identity thereto, or SEQ ID NO: 9 (AON3, a LNA+PS-modified sequence of SEQ ID NO: 8) or a nucleotide sequence having at least 75% sequence identity thereto. Accordingly, the nucleic acid silencing molecule may comprise or consist of SEQ ID NO: 8 or a nucleotide sequence having at least 75% sequence identity thereto, or SEQ ID NO: 9 (AON3, a LNA+PS-modified sequence of SEQ ID NO: 8) or a nucleotide sequence having at least 75% sequence identity thereto.

A nucleic acid silencing molecule that comprises or consists of SEQ ID NO: 10 or a nucleotide sequence having at least 75% sequence identity thereto may, for example, comprise or consist of SEQ ID NO: 11 or a nucleotide sequence having at least 75% sequence identity thereto, or SEQ ID NO: 12 (AON4, a 2′OMe+PS-modified sequence of SEQ ID NO: 11) or a nucleotide sequence having at least 75% sequence identity thereto, or SEQ ID NO: 13 (AON10, a LNA+PS-modified sequence of SEQ ID NO: 11) or a nucleotide sequence having at least 75% sequence identity thereto. Accordingly, the nucleic acid silencing molecule may comprise or consist of SEQ ID NO: 11 or a nucleotide sequence having at least 75% sequence identity thereto, or SEQ ID NO: 12 (AON4, a 2′OMe+PS-modified sequence of SEQ ID NO: 11) or a nucleotide sequence having at least 75% sequence identity thereto, or SEQ ID NO: 13 (AON10, a LNA+PS-modified sequence of SEQ ID NO: 11) or a nucleotide sequence having at least 75% sequence identity thereto.

A nucleic acid silencing molecule that comprises or consists of SEQ ID NO: 14 or a nucleotide sequence having at least 75% sequence identity thereto may, for example, comprise or consist of SEQ ID NO: 15 or a nucleotide sequence having at least 75% sequence identity thereto, or SEQ ID NO: 16 (AON5, a2′OMe+PS-modified sequence of SEQ ID NO: 15) or a nucleotide sequence having at least 75% sequence identity thereto. Accordingly, the nucleic acid silencing molecule may comprise or consist of SEQ ID NO: 15 or a nucleotide sequence having at least 75% sequence identity thereto, or SEQ ID NO: 16 (AON5, a 2′OMe+PS-modified sequence of SEQ ID NO: 15) or a nucleotide sequence having at least 75% sequence identity thereto.

A nucleic acid silencing molecule that comprises or consists of SEQ ID NO: 17 or a nucleotide sequence having at least 75% sequence identity thereto may, for example, comprise or consist of SEQ ID NO: 18 or a nucleotide sequence having at least 75% sequence identity thereto, or SEQ ID NO: 19 (AON6, a 2′OMe+PS-modified sequence of SEQ ID NO: 18) or a nucleotide sequence having at least 75% sequence identity thereto. Accordingly, the nucleic acid silencing molecule may comprise or consist of SEQ ID NO: 18 or a nucleotide sequence having at least 75% sequence identity thereto, or SEQ ID NO: 19 (AON6, a 2′OMe+PS-modified sequence of SEQ ID NO: 18) or a nucleotide sequence having at least 75% sequence identity thereto.

A nucleic acid silencing molecule that comprises or consists of SEQ ID NO: 20 or a nucleotide sequence having at least 75% sequence identity thereto may, for example, comprise or consist of SEQ ID NO: 21 or a nucleotide sequence having at least 75% sequence identity thereto, or SEQ ID NO: 22 (AON7a, a 2′OMe+PS-modified sequence of SEQ ID NO: 21) or a nucleotide sequence having at least 75% sequence identity thereto, or SEQ ID NO: 23 (AON7b, a MOE+PS-modified sequence of SEQ ID NO: 21) or a nucleotide sequence having at least 75% sequence identity thereto. Accordingly, the nucleic acid silencing molecule may comprise or consist of SEQ ID NO: 21 or a nucleotide sequence having at least 75% sequence identity thereto, or SEQ ID NO: 22 (AON7a, a 2′OMe+PS-modified sequence of SEQ ID NO: 21) or a nucleotide sequence having at least 75% sequence identity thereto, or SEQ ID NO: 23 (AON7b, a MOE+PS-modified sequence of SEQ ID NO: 21) or a nucleotide sequence having at least 75% sequence identity thereto. SEQ ID NOs: 20, 21, 22 and 23 target exon 11 of HPD.

A nucleic acid silencing molecule that comprises or consists of SEQ ID NO: 24 or a nucleotide sequence having at least 75% sequence identity thereto may, for example, comprise or consist of SEQ ID NO: 25 or a nucleotide sequence having at least 75% sequence identity thereto, or SEQ ID NO: 26 (AON8, a 2′OMe+PS-modified sequence of SEQ ID NO: 25) or a nucleotide sequence having at least 75% sequence identity thereto. Accordingly, the nucleic acid silencing molecule may comprise or consist of SEQ ID NO: 25 or a nucleotide sequence having at least 75% sequence identity thereto, or SEQ ID NO: 26 (AON8, a 2′OMe+PS-modified sequence of SEQ ID NO: 25) or a nucleotide sequence having at least 75% sequence identity thereto.

A nucleic acid silencing molecule that comprises or consists of SEQ ID NO: 27 or a nucleotide sequence having at least 75% sequence identity thereto may, for example, comprise or consist of SEQ ID NO: 28 or a nucleotide sequence having at least 75% sequence identity thereto, or SEQ ID NO: 29 (AON9, a LNA+PS-modified sequence of SEQ ID NO: 28) or a nucleotide sequence having at least 75% sequence identity thereto. Accordingly, the nucleic acid silencing molecule may comprise or consist of SEQ ID NO: 28 or a nucleotide sequence having at least 75% sequence identity thereto, or SEQ ID NO: 29 (AON9, a LNA+PS-modified sequence of SEQ ID NO: 28) or a nucleotide sequence having at least 75% sequence identity thereto.

A nucleic acid silencing molecule that comprises or consists of SEQ ID NO: 30 or a nucleotide sequence having at least 75% sequence identity thereto may, for example, comprise or consist of SEQ ID NO: 31 or a nucleotide sequence having at least 75% sequence identity thereto, or SEQ ID NO: 32 (AON11, a LNA+PS-modified sequence of SEQ ID NO: 31) or a nucleotide sequence having at least 75% sequence identity thereto. Accordingly, the nucleic acid silencing molecule may comprise or consist of SEQ ID NO: 31 or a nucleotide sequence having at least 75% sequence identity thereto, or SEQ ID NO: 32 (AON11, a LNA+PS-modified sequence of SEQ ID NO: 31) or a nucleotide sequence having at least 75% sequence identity thereto.

A nucleic acid silencing molecule that comprises or consists of SEQ ID NO: 33 or a nucleotide sequence having at least 75% sequence identity thereto may, for example, comprise or consist of SEQ ID NO: 34 or a nucleotide sequence having at least 75% sequence identity thereto, or SEQ ID NO: 35 (AON12, a LNA+PS-modified sequence of SEQ ID NO: 34) or a nucleotide sequence having at least 75% sequence identity thereto. Accordingly, the nucleic acid silencing molecule may comprise or consist of SEQ ID NO: 34 or a nucleotide sequence having at least 75% sequence identity thereto, or SEQ ID NO: 35 (AON12, a LNA+PS-modified sequence of SEQ ID NO: 34) or a nucleotide sequence having at least 75% sequence identity thereto.

A nucleic acid silencing molecule that comprises or consists of SEQ ID NO: 45 or a nucleotide sequence having at least 75% sequence identity thereto may, for example, comprise or consist of SEQ ID NO: 46 or a nucleotide sequence having at least 75% sequence identity thereto, or SEQ ID NO: 47 (AON13, a MOE+PS-modified sequence of SEQ ID NO: 46) or a nucleotide sequence having at least 75% sequence identity thereto. Accordingly, the nucleic acid silencing molecule may comprise or consist of SEQ ID NO: 46 or a nucleotide sequence having at least 75% sequence identity thereto, or SEQ ID NO: 47 (AON13, a MOE+PS-modified sequence of SEQ ID NO: 46) or a nucleotide sequence having at least 75% sequence identity thereto.

A nucleic acid silencing molecule that comprises or consists of SEQ ID NO: 48 or a nucleotide sequence having at least 75% sequence identity thereto may, for example, comprise or consist of SEQ ID NO: 49 or a nucleotide sequence having at least 75% sequence identity thereto, or SEQ ID NO: 50 (AON14, a MOE+PS-modified sequence of SEQ ID NO: 49) or a nucleotide sequence having at least 75% sequence identity thereto. Accordingly, the nucleic acid silencing molecule may comprise or consist of SEQ ID NO: 49 or a nucleotide sequence having at least 75% sequence identity thereto, or SEQ ID NO: 50 (AON14, a MOE+PS-modified sequence of SEQ ID NO: 49) or a nucleotide sequence having at least 75% sequence identity thereto.

A nucleic acid silencing molecule that comprises or consists of SEQ ID NO: 51 or a nucleotide sequence having at least 75% sequence identity thereto may, for example, comprise or consist of SEQ ID NO: 52 or a nucleotide sequence having at least 75% sequence identity thereto, or SEQ ID NO: 53 (AON15, a MOE+PS-modified sequence of SEQ ID NO: 52) or a nucleotide sequence having at least 75% sequence identity thereto. Accordingly, the nucleic acid silencing molecule may comprise or consist of SEQ ID NO: 52 or a nucleotide sequence having at least 75% sequence identity thereto, or SEQ ID NO: 53 (AON15, a MOE+PS-modified sequence of SEQ ID NO: 52) or a nucleotide sequence having at least 75% sequence identity thereto.

A nucleic acid silencing molecule that comprises or consists of SEQ ID NO: 54 or a nucleotide sequence having at least 75% sequence identity thereto may, for example, comprise or consist of SEQ ID NO: 55 or a nucleotide sequence having at least 75% sequence identity thereto, or SEQ ID NO: 56 (AON16, a MOE+PS-modified sequence of SEQ ID NO: 55) or a nucleotide sequence having at least 75% sequence identity thereto. Accordingly, the nucleic acid silencing molecule may comprise or consist of SEQ ID NO: 55 or a nucleotide sequence having at least 75% sequence identity thereto, or SEQ ID NO: 56 (AON16, a MOE+PS-modified sequence of SEQ ID NO: 55) or a nucleotide sequence having at least 75% sequence identity thereto.

A nucleic acid silencing molecule that comprises or consists of SEQ ID NO: 57 or a nucleotide sequence having at least 75% sequence identity thereto may, for example, comprise or consist of SEQ ID NO: 58 or a nucleotide sequence having at least 75% sequence identity thereto, or SEQ ID NO: 59 (AON17, a MOE+PS-modified sequence of SEQ ID NO: 58) or a nucleotide sequence having at least 75% sequence identity thereto. Accordingly, the nucleic acid silencing molecule may comprise or consist of SEQ ID NO: 58 or a nucleotide sequence having at least 75% sequence identity thereto, or SEQ ID NO: 59 (AON17, a MOE+PS-modified sequence of SEQ ID NO: 58) or a nucleotide sequence having at least 75% sequence identity thereto.

A nucleic acid silencing molecule that comprises or consists of SEQ ID NO: 60 or a nucleotide sequence having at least 75% sequence identity thereto may, for example, comprise or consist of SEQ ID NO: 61 or a nucleotide sequence having at least 75% sequence identity thereto, or SEQ ID NO: 62 (AON18, a MOE+PS-modified sequence of SEQ ID NO: 61) or a nucleotide sequence having at least 75% sequence identity thereto. Accordingly, the nucleic acid silencing molecule may comprise or consist of SEQ ID NO: 61 or a nucleotide sequence having at least 75% sequence identity thereto, or SEQ ID NO: 62 (AON18, a MOE+PS-modified sequence of SEQ ID NO: 61) or a nucleotide sequence having at least 75% sequence identity thereto.

A nucleic acid silencing molecule that comprises or consists of SEQ ID NO: 36 or a nucleotide sequence having at least 75% sequence identity thereto may, for example, comprise or consist of SEQ ID NO: 37 (siRNA1) or a nucleotide sequence having at least 75% sequence identity thereto. Accordingly, the nucleic acid silencing molecule may comprise or consist of SEQ ID NO: 37 (siRNA1) or a nucleotide sequence having at least 75% sequence identity thereto.

A nucleic acid silencing molecule that comprises or consists of SEQ ID NO: 38 or a nucleotide sequence having at least 75% sequence identity thereto may, for example, comprise or consist of SEQ ID NO: 39 (siRNA2) or a nucleotide sequence having at least 75% sequence identity thereto. Accordingly, the nucleic acid silencing molecule may comprise or consist of SEQ ID NO: 39 (siRNA2) or a nucleotide sequence having at least 75% sequence identity thereto.

A nucleic acid silencing molecule that comprises or consists of SEQ ID NO: 40 or a nucleotide sequence having at least 75% sequence identity thereto may, for example, comprise or consist of SEQ ID NO: 41 (siRNA3) or a nucleotide sequence having at least 75% sequence identity thereto. Accordingly, the nucleic acid silencing molecule may comprise or consist of SEQ ID NO: 41 (siRNA3) or a nucleotide sequence having at least 75% sequence identity thereto. A nucleotide sequence having at least 75% sequence identity to SEQ ID NO: 2, 3, 5, 6, 8, 9, 11, 12, 13, 15, 16, 18, 19, 21, 22, 23, 25, 26, 28, 29, 31, 32, 34, 35, 37, 39, 41, 46, 47, 49, 50, 52, 53, 55, 56, 58, 59, 61 or 62 may comprise one or more nucleotide substitutions with respect to SEQ ID NO: 2, 3, 5, 6, 8, 9, 11, 12, 13, 15, 16, 18, 19, 21, 22, 23, 25, 26, 28, 29, 31, 32, 34, 35, 37, 39, 41, 46, 47, 49, 50, 52, 53, 55, 56, 58, 59, 61 or 62 respectively. For example, the nucleotide sequence may comprise one, two, three or four substitutions with respect to SEQ ID NO: 2, 3, 5, 6, 8, 9, 31, 32, 34 or 35 which are 16mers. The nucleotide sequence may comprise one, two, three or four substitutions with respect to SEQ ID NO: 46, 47, 52, 53, 55, 56, 58, 59, 61 or 62 which are 17mers. The nucleotide sequence may comprise one, two, three, four, or five substitutions with respect to SEQ ID NO: 11, 12, 13, 15, 16, 18, 19, 21, 22, 23, 25, 26, 28, 29, 49 or 50 which are 20mers. The nucleotide sequence may comprise one, two, three, four, five or six substitutions with respect to SEQ ID NO: 37, 39 or 41.

A nucleotide sequence having at least 75% sequence identity to SEQ ID NO: 2, 3, 5, 6, 8, 9, 11, 12, 13, 15, 16, 18, 19, 21, 22, 23, 25, 26, 28, 29, 31, 32, 34, 35, 37, 39, 41, 46, 47, 49, 50, 52, 53, 55, 56, 58, 59, 61 or 62 may comprise one or more nucleotide deletions with respect to SEQ ID NO: 2, 3, 5, 6, 8, 9, 11, 12, 13, 15, 16, 18, 19, 21, 22, 23, 25, 26, 28, 29, 31, 32, 34, 35, 37, 39, 41, 46, 47, 49, 50, 52, 53, 55, 56, 58, 59, 61 or62 respectively. For example, the nucleotide sequence may comprise one, two, three or four deletions with respect to SEQ ID NO: 2, 3, 5, 6, 8, 9, 31, 32, 34 or 35 which are 16mers. The nucleotide sequence may comprise one, two, three or four deletions with respect to SEQ ID NO: 46, 47, 52, 53, 55, 56, 58, 59, 61 or 62 which are 17mers. The nucleotide sequence may comprise one, two, three, four, or five deletions with respect to SEQ ID NO: 11, 12, 13, 15, 16, 18, 19, 21, 22, 23, 25, 26, 28, or 29 which are 20mers. The nucleotide sequence may comprise one, two, three, four, five or six deletions with respect to SEQ ID NO: 37, 39 or 41.

Any of SEQ ID NOs: 1, 2, 4, 5, 7, 8, 10, 11, 14, 15, 17, 18, 20, 21, 24, 25, 27, 28, 30, 31, 33, 34, 36, 37, 38, 39, 40, 45, 46, 48, 49, 51, 52, 54, 55, 57, 58, 60 and 61 may comprise one or more (such as two or more, three or more, four or more, five or more, six or more, seven or more, eight or more, nine or more, 10 or more, 11 or more, 12 or more, 13 or more, 14 or more, 15 or more, 16 or more, 17 or more, 18 or more, or 19 or more) 2′-O-methoxyethylribose (MOE) modified nucleotides or consist of 2′-O-methoxyethylribose (MOE) modified nucleotides. The 2′-O-methoxyethylribose (MOE) modified nucleotides may be present at any position(s) within SEQ ID NOs: 1, 2, 4, 5, 7, 8, 10, 11, 14, 15, 17, 18, 20, 21, 24, 25, 27, 28, 30, 31, 33, 34, 36, 37, 38, 39, 40, 45, 46, 48, 49, 51, 52, 54, 55, 57, 58, 60 and 61.

Any of SEQ ID NOs: 1, 2, 4, 5, 7, 8, 10, 11, 14, 15, 17, 18, 20, 21, 24, 25, 27, 28, 30, 31, 33, 34, 36, 37, 38, 39, 40, 45, 46, 48, 49, 51, 52, 54, 55, 57, 58, 60 and 61 may comprise one or more (such as two or more, three or more, four or more, five or more, six or more, seven or more, eight or more, nine or more, 10 or more, 11 or more, 12 or more, 13 or more, 14 or more, 15 or more, 16 or more, 17 or more, 18 or more, or 19 or more) 2′-O-methyl (2OMe) modified nucleotides or consist of 2′-O-methyl (2OMe) modified nucleotides. The 2′-O-methyl (2OMe) modified nucleotides may be present at any position(s) within SEQ ID NOs: 1, 2, 4, 5, 7, 8, 10, 11, 14, 15, 17, 18, 20, 21, 24, 25, 27, 28, 30, 31, 33, 34, 36, 37, 38, 39, 40, 45, 46, 48, 49, 51, 52, 54, 55, 57, 58, 60 and 61.

Any of SEQ ID NOs: 1, 2, 4, 5, 7, 8, 10, 11, 14, 15, 17, 18, 20, 21, 24, 25, 27, 28, 30, 31, 33, 34, 36, 37, 38, 39, 40, 45, 46, 48, 49, 51, 52, 54, 55, 57, 58, 60 and 61 may comprise one or more (such as two or more, three or more, four or more, five or more, six or more, seven or more, eight or more, nine or more, 10 or more, 11 or more, 12 or more, 13 or more, 14 or more, 15 or more, 16 or more, 17 or more, 18 or more, or 19 or more) locked nucleic acid (LNA) modified nucleotides or consist of locked nucleic acid (LNA) modified nucleotides. The LNA modified nucleotides may be present at any position(s) within SEQ ID NOs: 1, 2, 4, 5, 7, 8, 10, 11, 14, 15, 17, 18, 20, 21, 24, 25, 27, 28, 30, 31, 33, 34, 36, 37, 38, 39, 40, 45, 46, 48, 49, 51, 52, 54, 55, 57, 58, 60 and 61.

Any of SEQ ID NOs: 1, 2, 4, 5, 7, 8, 10, 11, 14, 15, 17, 18, 20, 21, 24, 25, 27, 28, 30, 31, 33, 34, 36, 37, 38, 39, 40, 45, 46, 48, 49, 51, 52, 54, 55, 57, 58, 60 and 61 may comprise one or more (such as two or more, three or more, four or more, five or more, six or more, seven or more, eight or more, nine or more, 10 or more, 11 or more, 12 or more, 13 or more, 14 or more, 15 or more, 16 or more, 17 or more, 18 or more, or 19 or more) nucleotide phosphorothioates or consist of nucleotide phosphorothioates. The nucleotide phosphorothioate(s) may be present at any position(s) within SEQ ID NOs: 1, 2, 4, 5, 7, 8, 10, 11, 14, 15, 17, 18, 20, 21, 24, 25, 27, 28, 30, 31, 33, 34, 36, 37, 38, 39, 40, 45, 46, 48, 49, 51, 52, 54, 55, 57, 58, 60 and 61.

Any of SEQ ID NOs: 1, 2, 4, 5, 7, 8, 10, 11, 14, 15, 17, 18, 20, 21, 24, 25, 27, 28, 30, 31, 33, 34, 36, 37, 38, 39, 40, 45, 46, 48, 49, 51, 52, 54, 55, 57, 58, 60 and 61 may comprise any combination of (i) one or more 2OMe modified nucleotides, (ii) one or more LNA modified nucleotides, (iii) one or more 2′-O-methoxyethylribose (MOE) modified nucleotides and (iv) one or more nucleotide phosphorothioates. For example, any of SEQ ID NOs: 1, 2, 4, 5, 7, 8, 10, 11, 14, 15, 17, 18, 20, 21, 24, 25, 27, 28, 30, 31, 33, 34, 36, 37, 38, 39, 40, 45, 46, 48, 49, 51, 52, 54, 55, 57, 58, 60 and 61 may comprise (i); (ii); (iii); (iv); (i) and (ii); (i) and (iii); (i) and (iv); (ii) and (iii); (ii) and (iv); (iii) and (iv); (i), (ii) and (iii); (i), (ii) and (iv); (i), (iii) and (iv); (ii), (iii) and (iv); or (i), (ii), (iii) and (iv).

For illustrative purposes, exemplary LNA modification of SEQ ID NOs: 1, 4, 7, 10, 27, 30, and 33 is shown in SEQ ID NOs: 3, 6, 9, 13, 29, 32, and 35 respectively. Exemplary LNA modification of SEQ ID NOs: 2, 5, 8, 11, 28, 31, and 34 is shown in SEQ ID NOs: 3, 6, 9, 13, 29, 32, and 35 respectively. Exemplary nucleotide phosphorothioate modification of SEQ ID NOs: 1, 4, 7, 10, 14, 17, 20, 24, 27, 30, 33, 45, 48, 51, 54, 57 and 60 is shown in SEQ ID NOs: 3, 6, 9, 12 and 13, 16, 19, 22 and 23, 26, 29, 32, 35, 47, 50, 53, 56, 59 and 62 respectively. Exemplary nucleotide phosphorothioate modification of SEQ ID NOs: 2, 5, 8, 11, 15, 18, 21, 25, 28, 31, and 34 is shown in SEQ ID NOs: 3, 6, 9, 12 and 13, 16, 19, 22 and 23, 26, 29, 32, and 35 respectively. Exemplary 2′-O-methoxyethylribose (MOE) modification of SEQ ID NO: 20 is shown in SEQ ID NO: 23. Exemplary 2′-O-methoxyethylribose (MOE) modification of SEQ ID NOs: 21, 45, 48, 51, 54, 57 and 60 is shown in SEQ ID NOs: 23, 47, 50, 53, 56, 59 and 62. Exemplary 2′-O-methyl (2OMe) modification of SEQ ID NOs: 10, 14, 17, 20, and 24 is shown in SEQ ID NOs: 12, 16, 19, 22, and 26 respectively. Exemplary 2′-O-methyl (2OMe) modification of SEQ ID NOs: 11, 15, 18, 21, and 25 is shown in SEQ ID NOs: 12, 16, 19, 22, and 26 respectively. The exemplified modifications are not limiting. Any type of chemical modification (for example, 2OMe modification, LNA modification, 2′-O-methoxyethylribose (MOE) modification, nucleotide phosphorothioate modification) may be made at any or all of the exemplified positions. Chemical modifications (for example, 2OMe modification, LNA modification, 2′-O-methoxyethylribose (MOE) modification, nucleotide phosphorothioate modification) may also be made at non-exemplified positions.

In a preferred aspect, the nucleic acid silencing molecule comprises or consists of SEQ ID NO: 1 or a nucleotide sequence having at least 75% sequence identity thereto. In a more preferred aspect, the nucleic acid silencing molecule comprises or consists of SEQ ID NO: 2 or a nucleotide sequence having at least 75% sequence identity thereto. The nucleic acid silencing molecule may, for example, comprise or consist of a chemically modified version of SEQ ID NO: 2 (or a nucleotide sequence having at least 75% sequence identity thereto), such as SEQ ID NO: 3/AON1 (or a nucleotide sequence having at least 75% sequence identity thereto).

In another preferred aspect, the nucleic acid silencing molecule comprises or consists of SEQ ID NO: 20 or a nucleotide sequence having at least 75% sequence identity thereto. In a more preferred aspect, the nucleic acid silencing molecule comprises or consists of SEQ ID NO: 21 or a nucleotide sequence having at least 75% sequence identity thereto. The nucleic acid silencing molecule may, for example, comprise or consist of a chemically modified version of SEQ ID NO: 21 (or a nucleotide sequence having at least 75% sequence identity thereto), such as SEQ ID NO: 22/AON7a (or a nucleotide sequence having at least 75% sequence identity thereto) or SEQ ID NO: 23/AON7b (or a nucleotide sequence having at least 75% sequence identity thereto).

In a further preferred aspect, the nucleic acid silencing molecule comprises or consists of SEQ ID NO: 30 or a nucleotide sequence having at least 75% sequence identity thereto. In a more preferred aspect, the nucleic acid silencing molecule comprises or consists of SEQ ID NO: 31 or a nucleotide sequence having at least 75% sequence identity thereto. The nucleic acid silencing molecule may, for example, comprise or consist of a chemically modified version of SEQ ID NO: 31 (or a nucleotide sequence having at least 75% sequence identity thereto), such as SEQ ID NO: 32/AON11 (or a nucleotide sequence having at least 75% sequence identity thereto).

In another preferred aspect, the nucleic acid silencing molecule comprises or consists of SEQ ID NO: 45 or a nucleotide sequence having at least 75% sequence identity thereto. In a more preferred aspect, the nucleic acid silencing molecule comprises or consists of SEQ ID NO: 46 or a nucleotide sequence having at least 75% sequence identity thereto. The nucleic acid silencing molecule may, for example, comprise or consist of a chemically modified version of SEQ ID NO: 46 (or a nucleotide sequence having at least 75% sequence identity thereto), such as SEQ ID NO: 47/AON13 (or a nucleotide sequence having at least 75% sequence identity thereto). SEQ ID NOs: 45, 46 and 47 target exon 11 of HPD.

In another preferred aspect, the nucleic acid silencing molecule comprises or consists of SEQ ID NO: 48 or a nucleotide sequence having at least 75% sequence identity thereto. In a more preferred aspect, the nucleic acid silencing molecule comprises or consists of SEQ ID NO: 49 or a nucleotide sequence having at least 75% sequence identity thereto. The nucleic acid silencing molecule may, for example, comprise or consist of a chemically modified version of SEQ ID NO: 49 (or a nucleotide sequence having at least 75% sequence identity thereto), such as SEQ ID NO: 50/AON14 (or a nucleotide sequence having at least 75% sequence identity thereto). SEQ ID NOs: 48, 49 and 50 target exon 11 of HPD.

In another preferred aspect, the nucleic acid silencing molecule comprises or consists of SEQ ID NO: 51 or a nucleotide sequence having at least 75% sequence identity thereto. In a more preferred aspect, the nucleic acid silencing molecule comprises or consists of SEQ ID NO: 52 or a nucleotide sequence having at least 75% sequence identity thereto. The nucleic acid silencing molecule may, for example, comprise or consist of a chemically modified version of SEQ ID NO: 52 (or a nucleotide sequence having at least 75% sequence identity thereto), such as SEQ ID NO: 53/AON15 (or a nucleotide sequence having at least 75% sequence identity thereto). SEQ ID NOs: 51, 52 and 53 target exon 11 of HPD.

In another preferred aspect, the nucleic acid silencing molecule comprises or consists of SEQ ID NO: 54 or a nucleotide sequence having at least 75% sequence identity thereto. In a more preferred aspect, the nucleic acid silencing molecule comprises or consists of SEQ ID NO: 55 or a nucleotide sequence having at least 75% sequence identity thereto. The nucleic acid silencing molecule may, for example, comprise or consist of a chemically modified version of SEQ ID NO: 55 (or a nucleotide sequence having at least 75% sequence identity thereto), such as SEQ ID NO: 56/AON16 (or a nucleotide sequence having at least 75% sequence identity thereto). SEQ ID NOs: 54, 55 and 56 target exon 11 of HPD.

In another preferred aspect, the nucleic acid silencing molecule comprises or consists of SEQ ID NO: 57 or a nucleotide sequence having at least 75% sequence identity thereto. In a more preferred aspect, the nucleic acid silencing molecule comprises or consists of SEQ ID NO: 58 or a nucleotide sequence having at least 75% sequence identity thereto. The nucleic acid silencing molecule may, for example, comprise or consist of a chemically modified version of SEQ ID NO: 58 (or a nucleotide sequence having at least 75% sequence identity thereto), such as SEQ ID NO: 59/AON17 (or a nucleotide sequence having at least 75% sequence identity thereto). SEQ ID NOs: 57, 58 and 59 target exon 11 of HPD. In another preferred aspect, the nucleic acid silencing molecule comprises or consists of SEQ ID NO: 60 or a nucleotide sequence having at least 75% sequence identity thereto. In a more preferred aspect, the nucleic acid silencing molecule comprises or consists of SEQ ID NO: 61 or a nucleotide sequence having at least 75% sequence identity thereto. The nucleic acid silencing molecule may, for example, comprise or consist of a chemically modified version of SEQ ID NO: 61 (or a nucleotide sequence having at least 75% sequence identity thereto), such as SEQ ID NO: 62/AON18 (or a nucleotide sequence having at least 75% sequence identity thereto). SEQ ID NOs: 60, 61 and 62 target exon 11 of HPD.

### Conjugation

The nucleic acid silencing molecule may be conjugated to one or more non-nucleic acid moieties. For example, the nucleic acid silencing molecule may be conjugated to two or more, three or more, four or more, or five or more non-nucleic-acid moieties.

Preferably, the non-nucleic acid moiety is a delivery moiety. A delivery moiety is a molecule that assists in the in vivo delivery of a nucleic acid silencing molecule. For example, the delivery moiety may facilitate delivery of a nucleic acid silencing molecule to a target organ, tissue, or cell type. Any of the non-nucleic acid moieties mentioned below may function as a delivery moiety.

The non-nucleic acid moiety may, for example, be hydrophobic. The non-nucleic acid moiety may, for example, comprise a lipid. For example, the non-nucleic acid moiety may comprise cholesterol, a fatty acid, a triglyceride or a phospholipid. The fatty acid may, be saturated or unsaturated. The unsaturated fatty acid may be polyunsaturated. Preferably, the non-nucleic acid moiety comprises cholesterol or a polyunsaturated fatty acid.

The non-nucleic acid moiety may, for example, comprise a peptide, a polypeptide, or a protein. For example, the non-nucleic acid moiety may comprise a peptide ligand. The peptide or peptide ligand, may, for example, be a cognate ligand for a receptor present on the surface of a target cell, or a cell comprised in a target tissue or target organ. The non-nucleic acid moiety may, for example, comprise an antibody or antibody fragment. The antibody or antibody fragment, may, for example, be capable of binding to an antigen present on the surface of a target cell, or a cell comprised in a target tissue or organ. The antibody fragment may, for example, comprise or consist of a scFv, a Fab, a modified Fab, a Fab′, a modified Fab′, a F(ab′)2, or a scFv2.

The non-nucleic acid moiety may, for example, comprise a saccharide, disaccharide or polysaccharide. The non-nucleic acid moiety may, for example, comprise an amino sugar. Preferably, the non-nucleic acid moiety comprises N-Acetylgalactosamine (GalNAc). GalNAc is capable of binding to the asialoglycoprotein receptor (ASGPR), which is expressed on the surface of liver hepatocytes. Thus, by conjugating the nucleic acid silencing molecule to GalNAc, it is possible to deliver the nucleic acid silencing molecule specifically to liver hepatocytes. GalNAc is highly potent and has been demonstrated to dramatically increase the uptake of nucleic acid silencing molecules by hepatocytes, and to prolong its duration. Studies in mice have also suggested some biodistribution of GalNAc-conjugated oligonucleotides in the kidney. As tyrosine catabolism occurs primarily within the liver and then the kidney, conjugation of the nucleic acid silencing molecule to GalNAc is advantageous.

The non-nucleic acid moiety may, for example, comprise a nanoparticle. Suitable nanoparticles are known in the art. Methods for the production of such nanoparticles are also known. The nanoparticle may, for example, be a lipid nanoparticle, a liposome, polymeric nanoparticle, an inorganic nanoparticle, a virus-like particle (VLP), a self-assembling protein, a calcium phosphate nanoparticle, a silicon nanoparticle or a gold nanoparticle.

In one preferred aspect of the disclosure, the nucleic acid silencing molecule is conjugated to GalNAc. In another preferred aspect of the disclosure, the nucleic acid silencing molecule is conjugated to a lipid nanoparticle, such as a liposome. The nucleic acid silencing molecule may be conjugated to (i) GalNAc and (ii) a lipid nanoparticle, such as a liposome. Conjugation to GalNAc or a lipid nanoparticle (e.g. liposome) enhances the biodistribution and uptake of the nucleic acid silencing molecule in the liver and the kidney. As set out above, the liver and kidney are key target organs in the treatment of HT1. Conjugation to GalNAc or a lipid nanoparticle ensure that HPD is suppressed in these target organs to a sufficient extent to eliminate the accumulation of toxic metabolites systemically. Furthermore, HPD can be suppressed within every hepatocyte, preventing the formation of a mosaic liver that would lead to the development of HCC long-term.

### Method of Treatment and Medical Use

Disclosed herein is a method of treating a disorder of the tyrosine degradation pathway in a subject, comprising administering to the subject a composition comprising a nucleic acid silencing molecule that reduces the expression of 4-hydroxyphenylpyruvate dioxygenase (HPD).

Also disclosed herein is a composition for use in a method of treating a disorder of the tyrosine degradation pathway in a subject, wherein the composition comprises a nucleic acid silencing molecule that reduces the expression of 4-hydroxyphenylpyruvate dioxygenase (HPD), and the method comprises administering the nucleic acid silencing molecule to the subject.

Administration of a nucleic acid silencing molecule that reduces the expression of HPD to a subject having a disorder of the tyrosine degradation pathway may block or inhibit the pathway at its early steps. In this way, the formation of downstream metabolites may be prevented or reduced. Accumulation of toxic metabolites, such as hepatotoxic and/or nephrotoxic metabolites, may thus be prevented or reduced. By preventing or reducing the accumulation of toxic metabolites, disorders of the tyrosine degradation pathway may be treated. For example, clinical signs or symptoms of such disorders may be reduced or abolished. Clinical signs or symptoms may be stopped from progressing. The method of treatment and medical use disclosed herein may have advantages over other treatments for disorders of the tyrosine degradation pathway, such as NTBC administration, as set out above.

### Nucleic Acid Silencing Molecule

The method of treatment and medical use described herein comprise administering to the subject a composition comprising a nucleic acid silencing molecule that reduces the expression of HPD. Such nucleic acid silencing molecules are described in detail above. Any of the aspects described above in connection with the nucleic acid silencing molecule of the disclosure may equally apply to the method of treatment of the disclosure or to the medical use of the disclosure.

The composition may comprise one or more nucleic acid silencing molecules that reduce the expression of HPD. For example, the composition may comprise two or more, five or more, ten or more, 20 or more, 50 or more, 100 or more, 200 or more, 500 or more, 1000 or more, 2000 or more, 5000 or more, 10000 or more, 20000 or more, 50000 or more, 100000 or more, 200000 or more, 500000 or more, 1000000 or more, 2000000 or more, 5000000 or more, 1×10⁷ or more, 2×10⁷ or more, 5×10⁷ or more, 1×10⁸ or more, 2×10⁸ or more, 5×10⁸ or more, 1×10⁹ or more, 2×10⁹ or more, or 5×10⁹ or more nucleic acid silencing molecules that reduce the expression of HPD per dose. Preferably, all of the more nucleic acid silencing molecules comprised in one dose of the composition comprise the same nucleotide sequence.

In one aspect, the nucleic acid silencing molecule may comprise one or more nucleotide phosphorothioates, or consist of nucleotide phosphorothioates. When the composition comprises two or more such nucleic acid silencing molecules, each of the two or more nucleic acid silencing molecules may be of one stereoisomer. Preferably, the composition comprises no further nucleic acid silencing molecules. This ensures the stereopurity of the composition. Stereopurity of nucleic acid silencing molecules is described in the art, for example in Iwamoto et al. (2017), Nature Biotechnology, 35:9, 845-851.

### Reducing the Expression of HPD

The nucleic acid silencing molecule comprised in the composition reduces the expression of HPD. Reduced expression of HPD is described in detail above in connection with the nucleic acid silencing molecule of the disclosure. Any of the aspects described in connection with the nucleic acid silencing molecule of the disclosure may equally apply to the method of treatment of the disclosure or to the medical use of the disclosure.

### Disorders of the Tyrosine Degradation Pathway

Administration of a nucleic acid silencing molecule that reduces the expression of HPD blocks or inhibits the tyrosine degradation pathway at its early steps, because HPS is the second enzyme in the pathway. By blocking or inhibiting the top of the pathway upstream in this way, the formation of any downstream metabolites may be prevented or reduced. Therefore, any disorder of the tyrosine degradation pathway downstream of HPD may be treated according to the method of treatment or medical use of the disclosure.

Preferably, the disorder is associated with the accumulation of toxic metabolites of the tyrosine degradation pathway. The toxic metabolites may, for example, be hepatotoxic metabolites and/or nephrotoxic metabolites. Preferably, the disorder comprises a deficiency in one or more (such as two or more, three or more, or four or more) of the enzymes comprised in the tyrosine degradation pathway. The disorder may, for example, comprise a deficiency in fumarylacetoacetate hydrolase (FAH). The disorder may, for example, comprise a deficiency in homogentisate 1,2-dioxygenase (HGD).

The deficiency in the one or more enzymes may result from a mutation in the enzyme. For example, the deficiency in FAH may result from a mutation in FAH. The deficiency in HGD may result from a mutation in HGD. The mutation may comprise one or more substitutions in the enzyme. The mutation may comprise one or more insertions to the enzyme. The mutation may comprise one or more deletions from the enzyme.

The disorder may, for example, be Hereditary Tyrosinemia Type I (HT1). Pyridoxine-dependent epilepsy results from a deficiency (e.g. a mutation) in FAH. The disorder may, for example, be Alkaptonuria (AKU). AKU results from a deficiency (e.g. a mutation) in HGD.

### Subject

The subject may, for example, be a mammal. The mammal may, for example, be a human or a non-human mammal such as a dog, cat, horse or farm animal. Preferably, the subject is a human.

The subject may, for example, be an adult. The subject may, for example, be a juvenile.

### Administration and Formulation

The composition may be administered by any route. Suitable routes include, but are not limited to, the intravenous, intrathecal, intracerebral ventricular, intramuscular, intraperitoneal, subcutaneous, intradermal, transdermal and oral/buccal routes.

The composition may comprise a delivery vehicle that optimises delivery of the nucleic acid silencing molecule in vivo. Suitable delivery vehicles are known in the art and include, for example, cell-targeting moieties, cell-penetrating moieties, lipids, lipoproteins, liposomes, lipoplexes, peptides, GalNAc, antibodies, aptamers, nanoparticles, exosomes, spherical nucleic acids, and DNA cages.

The compositions may be prepared together with a physiologically acceptable carrier or diluent. Typically, such compositions are prepared as liquid suspensions of nucleic acid silencing molecules and/or delivery vehicle-linked nucleic acid silencing molecules. The nucleic acid silencing molecules and/or delivery vehicle-linked nucleic acid silencing molecules may be mixed with an excipient which is pharmaceutically acceptable and compatible with the active ingredient. Suitable excipients are, for example, water, saline, dextrose, glycerol, of the like and combinations thereof. In addition, if desired, the pharmaceutical compositions may contain minor amounts of auxiliary substances such as wetting or emulsifying agents, and/or pH buffering agents.

The nucleic acid silencing molecules and/or delivery vehicle-linked nucleic acid silencing molecules are administered in a manner compatible with the dosage formulation and in such amount will be therapeutically effective. The quantity to be administered depends on the subject to be treated, the disease to be treated, and the capacity of the subject's immune system. Precise amounts of nucleic acid silencing molecules and/or delivery vehicle-linked nucleic acid silencing molecules required to be administered may depend on the judgement of the practitioner and may be peculiar to each subject.

As set out above, the nucleic acid silencing molecule may, in one aspect, be a CRISPR guide RNA. In this case, a CRISPR nuclease (such as Cas9, Cpf1, Cas12b, or CasX) may be administered to the subject. The CRISPR nuclease may be comprised in the composition comprising the nucleic acid silencing molecule, or in a separate composition. If the CRISPR nuclease is comprised in a separate composition, the composition comprising the nucleic acid silencing molecule and the composition comprising the CRISPR nuclease may be administered to the subject at the same time. The composition comprising the nucleic acid silencing molecule and the composition comprising the CRISPR nuclease may be administered to the subject at a different time. For example, the composition comprising the nucleic acid silencing molecule may be administered to the subject before the composition comprising the CRISPR nuclease. The composition comprising the nucleic acid silencing molecule may be administered to the subject after the composition comprising the CRISPR nuclease.

### Combination Therapy

The composition may be administered as part of a combination therapy. That is, the method of treatment or medical use may comprise administering to the subject a further therapeutic composition or a therapeutic regimen. Administration of a further composition or a therapeutic regimen may, for example, be desirable when the nucleic acid silencing molecule reduces rather than eliminates HPD expression. In some cases, though, reduction (rather than elimination) of HPD expression may be sufficient to effect treatment of the disorder.

The composition may be administered as part of a combination therapy in conjunction with any available therapeutic composition or therapeutic regimen for a particular disorder. For instance, disorders of the tyrosine degradation pathway are typically treated by administering NTBC. Thus, the further therapeutic composition may comprise NTBC. Disorders of the tyrosine degradation pathway may be treated with a protein-restricted diet, such as a diet that is restricted in tyrosine and/or phenylalanine. Thus, the therapeutic regimen may comprise a protein-restricted (e.g. tyrosine and/or phenylalanine restricted) diet.

In one aspect of combination therapy, the composition that comprises a nucleic acid silencing molecule that reduces the expression of HPD is be administered in such an amount that will be therapeutically effective in combination with administration of the further therapeutic composition. The further therapeutic composition is administered, in such an amount that will be therapeutically effective in combination with the composition that comprises a nucleic acid silencing molecule that reduces the expression of HPD. The composition that comprises a nucleic acid silencing molecule that reduces the expression of HPD and the further therapeutic composition may be administered together, for instance at the same time. The composition that comprises a nucleic acid silencing molecule that reduces the expression of HPD and the further therapeutic composition may be administered separately, for instance at a different time. For example, the composition that comprises a nucleic acid silencing molecule that reduces the expression of HPD may be administered before the further therapeutic composition. The composition that comprises a nucleic acid silencing molecule that reduces the expression of HPD may be administered after the further therapeutic composition. Administration of the composition that comprises a nucleic acid silencing molecule that reduces the expression of HPD may be alternated with administration of the further therapeutic composition.

In another aspect of combination therapy, the composition that comprises a nucleic acid silencing molecule that reduces the expression of HPD is administered in such an amount that it will be therapeutically effective in combination with the additional therapeutic regimen. The therapeutic regimen is implemented to the extent that it will be therapeutically effective in combination with administration of the composition that comprises a nucleic acid silencing molecule that reduces the expression of HPD. The composition that comprises a nucleic acid silencing molecule that reduces the expression of HPD may be administered before, during or after implementation of the therapeutic regimen. Preferably, the composition that comprises a nucleic acid silencing molecule that reduces the expression of HPD is administered during implementation of the therapeutic regimen.

### In Vitro Method

As set out above, the nucleic acid silencing molecule disclosed herein may be used to reduce the expression of HPD in vitro. For example, the nucleic acid silencing molecule may be used as a research tool, for instance to investigate the tyrosine degradation pathway. Accordingly, the present disclosure provides an in vitro method for reducing expression of HPD in a cell, comprising contacting the cell with the nucleic acid silencing molecule of the disclosure.

The cell may by any type of cell. For example, the cell may be from any tissue. The cell may be from any species. Preferably, the cell is human. The cell may be a healthy cell, or a diseased cell. The diseased cell may, for example, be a cancer cell. The cell may be a naturally occurring cell. The cell may be from a cell line. The cell may, for example be a liver cell, or from a liver cell line. The cell may, for example be a kidney cell, or from a kidney cell line. The cell may, for example be a human liver cell, or from a human liver cell line. The cell may, for example be a human kidney cell, or from a human kidney cell line.

Mechanisms for contacting a cell with a nucleic acid silencing molecule are well known in the art. Contacting may, for example, take place in a well of a microwell plate, or in another type of vessel such as a cell culture flask or a test tube. The contacting step may be performed prior to, or concurrently with, culture of the cell. The conditions required for the culture of various cell types are well known in the art.

The cell may be contacted with one or more molecules additional to the nucleic acid silencing molecule. The additional molecule may be another nucleic acid silencing molecule that targets HPD. The additional molecule may be a nucleic acid silencing molecule that targets a gene other than HPD, such as a different gene within the tyrosine degradation pathway. Preferably, the additional molecule may facilitate reduction or elimination of HPD expression by the nucleic acid silencing molecule. For example, if the nucleic acid silencing molecule is a CRISPR guide RNA, the additional molecule may be a CRISPR nuclease. The CRISPR nuclease may, for example, be Cas9, Cpf1, Cas12b, or CasX.

The following Examples illustrate the invention.

### EXAMPLES

### Example 1

### Materials and Methods

Human HepG2 Cell culture

Cells were grown in Minumum Essential Medium (MEM) supplemented with 10% heat inactivated fetal bovine serum (FBS), at 37° C. and 5% CO₂.

Mouse Fah^{-/-} mice Fah^{-/-} mice with complete loss of Fah activity were used as an animal model of acute HT1. Mice were maintained with NTBC added in drinking water.

### Antisense Oligonucleotides (AONs) Design

AONs with Locked Nucleic Acid (LNA) (Qiagen) and 2′-O-methoxy-ethyl (MOE) (Eurogentec) chemistries were designed to target HPD (4 hydroxyphenylpyruvate dioxygenase) and synthesised commercially. The length of gapmer was 16 mers for LNA chemistry and 20 mers for MOE chemistry. The central DNA gap is phosphorothioate (PS) modified DNA, while the flanking RNA regions were modified with MOE or LNA. GalNAc-AON conjugation was commercially synthesized by Axolabs in Germany.

AON or siRNA Treatment of Cells

Reverse Transfection was used for transfecting HepG2 cells. Cells were seeded in a six-well plate at a density of 5×10⁴ cells/cm² in growth medium (1.5 mL) together with 2.5 μL Lipofectamine 2000 (ThermoFisher) containing AON at the desired concentration, or 5 μL RNAiMAX (ThermoFisher) containing siRNA at the desired concentration, and cultured for 24 hours. For each experiment three control conditions were used: cells treated only with lipofectamine (mock); cells treated with an AON or siRNA with a scrambled sequence that does not bind to mRNA (scrambled); and cells treated only with growth media (blank).

RNA Extraction and cDNA Preparation

Total RNA was extracted from cells using the RNeasy Plus Mini Kit according to manufacturer's instructions. The yield and quality of the RNA of each sample was determined by measuring the absorbance at 260 and 280 nm using a NanoDrop spectrophotometer. Total RNA (400-500 ng) was reverse transcribed, according to the manufacturer instructions, in a 20 μL reaction mixture using High-Capacity RNA-to-cDNA Synthesis kit (ThermoFisher).

### Quantitative Real-Time PCR (RT-PCR) Analysis

Quantitative real-time PCR analysis was performed using SYBR™ Green PCR Master Mix (Eurogentec). Optimized Primers for HPD and RPL19 genes are detailed below. The StepOne™ real-time PCR system (Applied Biosystems) was used for quantitative real-time PCR and analysis using the following method: holding stage 95° C. for 10 min, followed by 40 cycles of denaturation at 95° C. for 15 seconds and annealing at 60° C. for 1 min, with melt curve (step 1: 95° C. for 15 seconds, step 2: 60° C. for 1 minute and step 3: 95° C. for 15 seconds (+0.3° C.). The relative quantification was measured using the ΔΔCt method. HPD expression levels were normalised to RPL19. Each sample was analysed in triplicate.

| Gene | Forward Primer | Reverse Primer |
|---|---|---|
| HPD | CCAGATCCAGGAATATGTG | CTCCAGGCCTCTCTCTCTCA |
| | GAC | |
| RPL19 | GCGGAAGGGTACAGCCAAT | CAGGCTGTGATACATGTGGCG |

### Quantification and Statistical Analysis

Data were analysed using a two-tailed unpaired Student's t-test (GraphPad Prism 8 software). When multiple groups were compared, a one-way ANOVA test was performed. p<0.05 was considered significant. Data are presented as mean±SEM.

### Results

AONs Decrease HPD mRNA Expression in HepG2 Cells

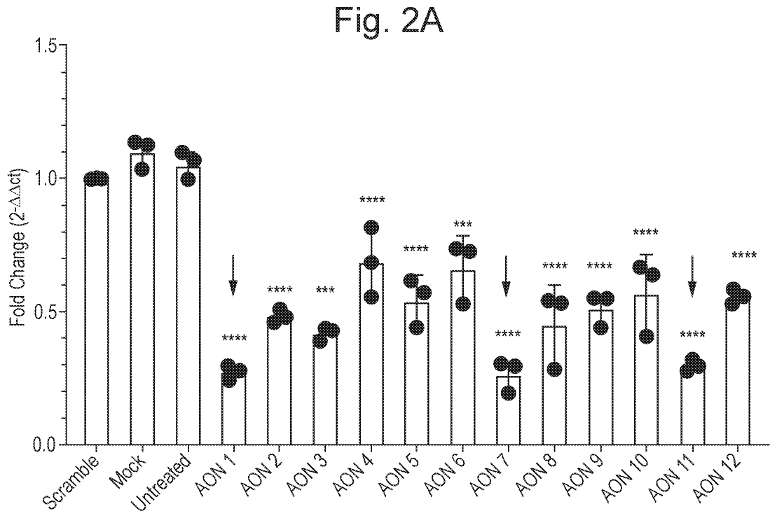

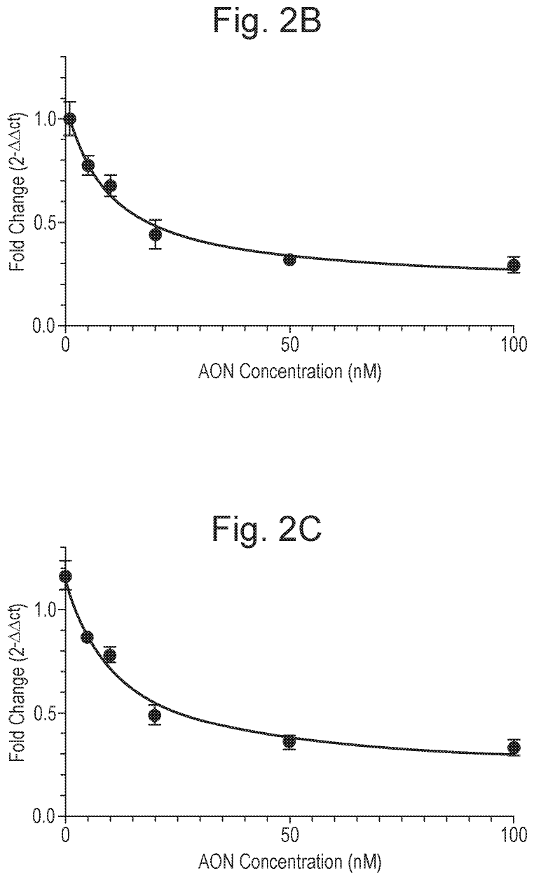

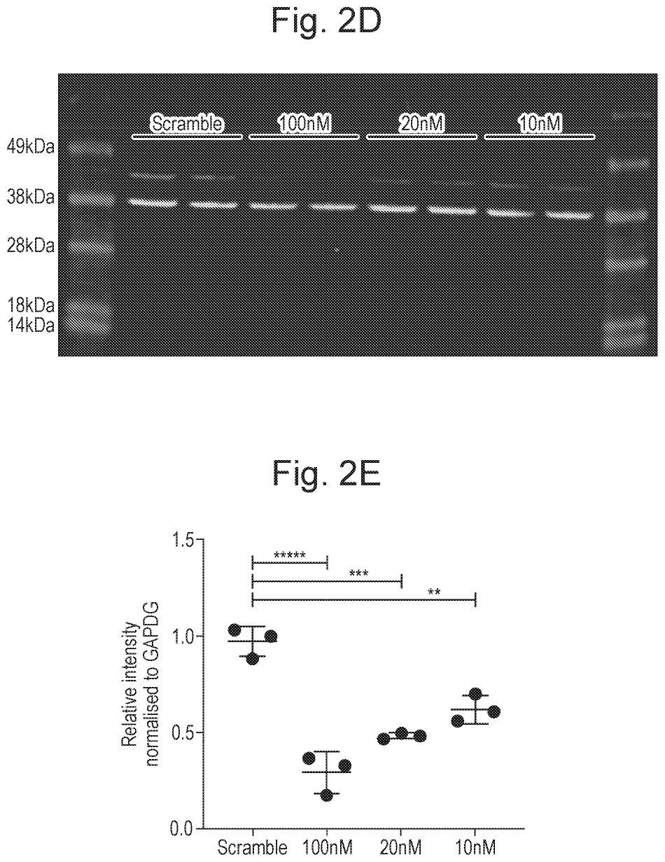

The effect of treatment with HPD-specific antisense oligonucleotide on HPD mRNA expression was assessed in human hepatocyte line HepG2. The results are shown in FIG. 2. Both LNA modified and MOE modified antisense oligonucleotides reduced expression of HPD mRNA. In particular, AON1 (LNA), 7 (MOE) and 11 (LNA) showing strong silencing effect (in 73%, 74% and 70% reduction, respectively, p=0.0001) (FIG. 2A). Representative examples of dose response curves of AONs 1 and 7 are shown in FIGS. 2B-C. AON1 has an IC50 of 9.5 nM (95% CI: 5.97-15-19 nM), with a maximal inhibition showing a fold change of 0.195 (95% CI: 0.10-0.27), indicating that the current designs are capable of reducing HPD expression by 80%. AON 7 shows a similar profile with an IC50 of 11.43 nM (95% CI: 8.10-16.38 nM) and a 10 nM HPD shows a fold change of 0.198 (80.2% reduction, 95% CI: 0.105-0.280). The results are shown in FIG. 2 A-C.

### AONs Decrease HPD Protein Expression in HepG2 Cells

The effect of AONs on HPD protein expression was assessed by western blotting in HepG2 cells in triplicate. Cells were treated with AON7-MOE at 100, 20 and 10 nM. Semi-quantification of western blotting showed reduction in HPD protein at around 71%, 52% and 38%, respectively (HPD, top band in red, relative to tubulin, lower band in green). The results are shown in FIGS. 2 D and E.

siRNAs Decrease HPD mRNA Expression in HepG2 Cells

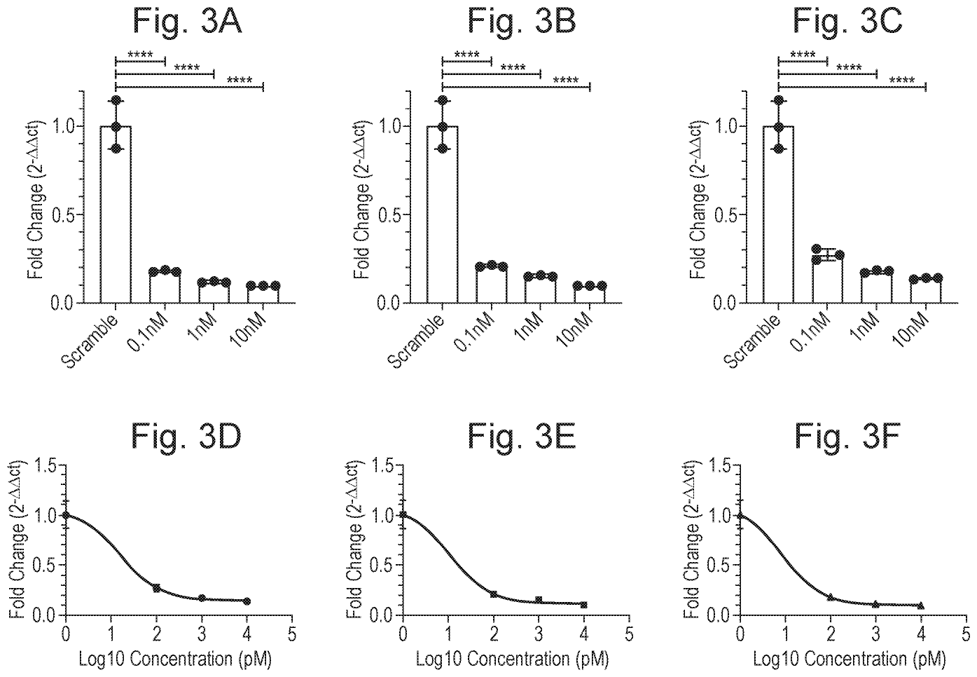

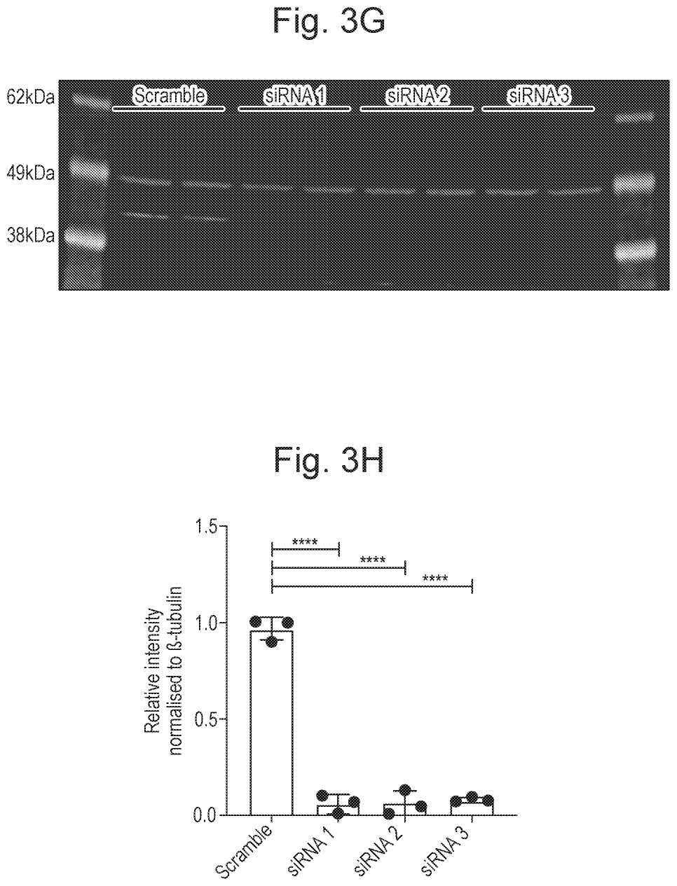

Representative data of 3 siRNA candidates suppressing HPD expression by up to 90% in HepG2s (FIG. 3). At 0.1 nM siRNAl reduces HPD expression to a fold-change of 0.2737 (73% reduction, SD: ±0.03) compared to scramble treated controls (p<0.0001), which is further reduced to 0.1431 (86% reduction, SD: ±0.006) at lOnM (p<0.0001). siRNA2 and 3 show maximum reductions in expression of 0.102 (90% reduction, SD: ±0.005) and 0.101 (90% reduction, SD: ±0.002) respectively at 10 nM compared to scramble controls (p<0.0001) at the RNA level. The results are shown in FIG. 3 A-F.

siRNAs Decrease HPD Protein Expression in HepG2 Cells

Assessments of HPD downregulation at the protein level confirms a strong effect for all siRNAs, confirming the strong effect of our current candidate siRNAs. siRNA 1 shows a 94% downregulation (SD: ±4.75%, p<0.0001); siRNA 2 shows a 94% downregulation (SD±6.206%, p<0.0001); and siRNA 3 shows a 92% downregulation (SD: ±1.294%, p<0.0001) compared to scramble treated controls. There are no significant differences between any of the tested siRNAs. The results are shown in FIG. 3 G-H

AONs Decrease Hpd mRNA and Protein Expression in Fah^{-/-} Mice

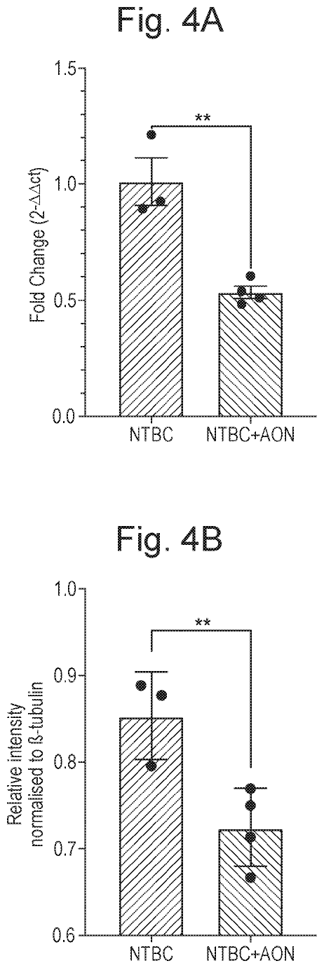

Neonatal Fah^{-/-} mice (under NTBC) received weekly subcutaneous injection of AON7 (MOE) at 50 μg/g for 5 weeks. Mice were culled one week after the last injection. Liver tissues were collected for the measurement of mouse Hpd mRNA and protein. The results are shown in FIG. 4. AON7 reduced Hpd mRNA and protein expression by approximately 50% (p<0.01).

GalNAc Conjugated AON7 Decrease Mouse Hpd mRNA Expression in Fah^{-/-} Mice

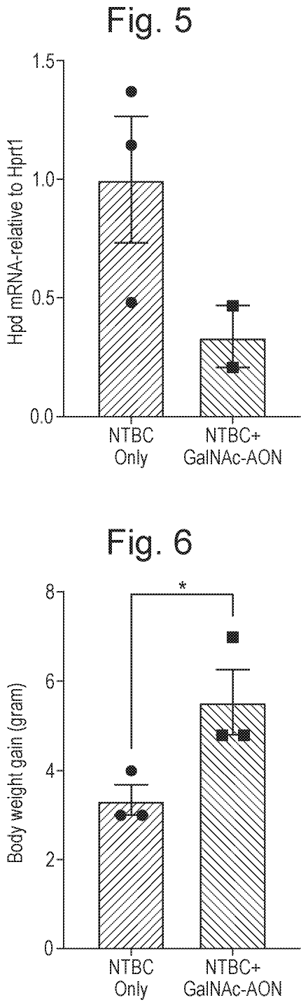

3-week-old Fah^{-/-} mice, maintained under NTBC drinking water, were given a single dose of GalNAc-AON at 5 μg/g subcutaneously. Liver tissues were collected one week after for the measurement of Hpd mRNA. Over 60% reduction in Hpd mRNA was detected in GalNAc-AON treated group (n=2) compared to the littermate control group under NTBC only (n=3). The result is shown in FIG. 5.

GalNAc Conjugated AON7 Increases Bodyweight in Combination with NTBC Treatment

3-week-old Fah^{-/-} mice, maintained under NTBC drinking water, were given a single dose of GalNAc-AON at 5 μg/g subcutaneously. The body weight gain during the one-week GalNAc-AON treatment (n=3, mean weight gain=5.5 g) was measured, and compared to the littermate control group under NTBC only (n=3, mean weight gain=3.3 g, p=0.026). Data represent mean±SEM. The result is shown in FIG. 6.

### GalNAc-AON Increases the Survival of Fah^{-/-} Mice

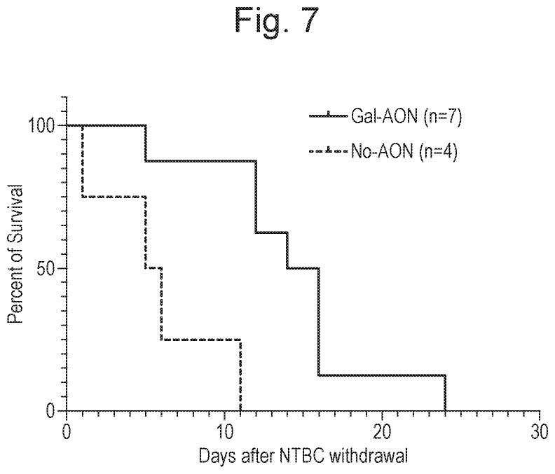

New-born Fah^{-/-} mice were given a single dose of GalNAc-AON7b at 5 μg/g subcutaneously. The injection was repeated every 2 weeks. NTBC water was withdrawn from both GalNAc-AON treated group and No-AON control group at 4 weeks old after the 3^{rd} injection. After withdrawing NTBC, Fah−/− mice lived significantly longer in GalNAc-AON treated group (mean=15 days, n=7) than in No-AON group (mean=5.5 days, n=4, p=0.0028). The result is shown in FIG. 7.

Sequence listing

SEQ ID NO: 1
GGCACGCNNNAANCGG
SEQ ID NO: 2
GGCACGCTTTAATCGG
SEQ ID NO: 3
{GGC}A*C*G*C*T*T*T*A*A*T*{CGG}
SEQ ID NO: 4
GAAACGANNAAGGANC
SEQ ID NO: 5
GAAACGATTAAGGAUC
SEQ ID NO: 6
{GAA}A*C*G*A*T*T*A*A*G*G*{AUC}
SEQ ID NO: 7
GAANNGGNCANCNNNG
SEQ ID NO: 8
GAATTGGTCATCTTTG
SEQ ID NO: 9
{GAA}T*T*G*G*T*C*A*T*C*T*{TTG}
SEQ ID NO: 10
CAGCAAACNNCACCNNCCCA
SEQ ID NO: 11
CAGCAAACTTCACCTUCCCA
SEQ ID NO: 12
<CAGCA>A*A*C*T*T*C*A*C*C*T*<UCCCA>
SEQ ID NO: 13
{CAGCA}A*A*C*T*T*C*A*C*C*T*{UCCCA}
SEQ ID NO: 14
CCANACGNCNGCAGCACAGC
SEQ ID NO: 15
CCAUACGTCTGCAGCACAGC
SEQ ID NO: 16
<CCAUA>C*G*T*C*T*G*C*A*G*C*< ACAGC>
SEQ ID NO: 17
NCCGNGNGCACCNGCGNGNC
SEQ ID NO: 18
UCCGUGTGCACCTGCGUGUC
SEQ ID NO: 19
<UCCGU>G*T*G*C*A*C*C*T*G*C*< GUGUC>
SEQ ID NO: 20
NCNNGAGAGCGANGNGCNGG
SEQ ID NO: 21
UCUUGAGAGCGATGTGCUGG
SEQ ID NO: 22
<UCUUG>A*G*A*G*C*G*A*T*G*T*<GCUGG>
SEQ ID NO: 23
[UCUUG]A*G*A*G*C*G*A*T*G*T*[GCUGG]
SEQ ID NO: 24
AGGAGGNAGCCNNNCNCGNC
SEQ ID NO: 25
AGGAGGTAGCCTTTCUCGUC
SEQ ID NO: 26
<AGGAG>G*T*A*G*C*C*T*T*T*C *<UCGUC>
SEQ ID NO: 27
NCGCCCANCNCNNNGNNCCA
SEQ ID NO: 28
UCGCCCATCTCTTTGUUCCA
SEQ ID NO: 29
{UCGCC}C*A*T*C*T*C*T*T*T*G*{UUCCA}
SEQ ID NO: 30
CACNGNAAGNCGNCAN
SEQ ID NO: 31
CACTGTAAGTCGTCAU
SEQ ID NO: 32
{CAC}T*G*T*A*A*G*T*C*G*T*{CAU}
SEQ ID NO: 33
CNGCAGNAGAANGACG
SEQ ID NO: 34
CUGCAGTAGAATGACG
SEQ ID NO: 35
{CUG}C*A*G*T*A*G*A*A*T*G*{ACG}
SEQ ID NO: 36
NGGNCAGCCANGNAANCAAACAAGGNN
SEQ ID NO: 37
TGGTCAGCCATGTAATCAAACAAGGdTdT
SEQ ID NO: 38
CGNNCGAGGNGGAAGANNGNGACNANN
SEQ ID NO: 39
CGTTCGAGGTGGAAGATTGTGACTAdTdT
SEQ ID NO: 40
GGNGGAAGANNGNGACNACANCGNNN
SEQ ID NO: 41
GGTGGAAGATTGTGACTACATCGTdTdT
SEQ ID NO: 42
TCTCAGCTCAATGAACCCTGCCTCCCGGGTTCAAGTGATTCTCCTGCCTCAGCC
TCCTGAGTAGCTAGGATTACAGGTGTGTACCACCATGCCCAGCTAATTTTTTTT
TTTTTTTTTTTTTTTGTATTTTTAGTAGAAACAGGGTTTCGCTATGTTGGCCAGG
CTGGTCTCAAATTCCTGACCTCAAATGATCTGCCTGCCTTGGCCTCCCAAAGTG
CTGGGATTACAGGCATGAGCCACCGTGCCCAGCCAAGAGGGACAAAAACCTTT
TCACAGAAAAACATTTTTATACAACTTAACCAGAAAGCTTCATTTCAGGTTCAG
GAAGTGGAAGGGGCAAAATCCTGGATTTTCACAAATTTCCAAAATGGGCTTAT
ATAACTCTCCACCCTTTTCTTTCTTTTTTTTTTTCTTTTAGAGACAGGGTCTCATT
CTGCTGCCCAGGCTGAAGTGCAGTGGTGCAATTATAGCTCACTGTACCCTTGA
ACTCCTGGGCTCAAGGGATCCCCCCATCTCAGCCTCTTAAAGTGCTGGGATTAC
AGGTGTCAGCCACTGCTCCTGGCTCGCCACCAATGCTTTTCATTCTGATCATTT
CTGTAGTGAGCAGCATAGAAGAAATAATGCCTTTTGAGGGACAGTTGCATTTA
ACAGACAGCCTTACTAAGGTGAGTACGTCAGGACAAAGATGCGGGAAGGAGA
ATCAGGCTATTAGTCCATTGTTGCAAATTGCAACGAATTGAGAAAGTCAGACA
CAACGATCAACTGCAGTGAAACATTTGGTGGGGAGTTTTGAGAGTCCAGTCTG
AAGGAGTGCGTGGAAGGATCACGGTCCCTGTGATGTACCCTCTCTCGGCTTGTT
GCTTTGGGCAGGAATCACAAGTTAGGAAGGAATCAGCACCAGAAATGCAGAG
CTGGCTGGGCGCAGTGGCTCACGCCTGTAATCCCAGCACTTTGGGAGGCCGAG
GCGGGAAGATCACCTGAGGCCAAGAGTTCGAGATCAGCCTCGCCAACATGGC
GAAGCCCTGTCTCTGATAAAAAATACAAAAATTAGCTTGGTGTGGTGGCGCAT
GCCTGTAGTCTTAACTACTTGGGAGACTGAGGCGGGAGAATCACTTGAACCTA
GGAGGCGGAGGTTGCAGTGAGCAGAGATCACGCCATTGCACTTCAGCTTGGGA
GACATCCCAAGACCATCTTAAAAAAAAAAAAAGGAGGGGGGGTGCCGGGCGC
GGTGGCTCACGCCTATAATCCCGGCACTTTGGGAGGCCAAGGCGAGCGAATCA
CGAGGTCAGGAGATTGAGACCATCCTGGCTAACACGGTGAAACCTCATCTCTA
CTAAAAATACAAAACATTAGCTGAGCCAGGTGGCAGGTGCCTGTATTCCCAGC
TACTCCGGAGGCTGAGGCAGGAGAATGGCGTGAACCTTGGAGGTGGAGCTTGC
AGTGAGCCGAGATTGCACCATTGCACTCCAGCCTAAAAAAAAAAAAAAAAAA
AAAAAAAGCTTGCATTGAGCCGAGATGCCAAGATCACGCCACTGCACTCCAGC
CTGGGCCACAGAGGGAGACTCTGTCTCAAAAAAAAAAAAAAAAAAAAAAAAA
AAAAGGCCGAGAGCGGTGGCTCACGCCTGTAATCCCAGCACTTTGGGAGGCCG
AGGTGGGCAGATCACCTGAGGTCAGGAGTTCGAGACATGCCTGACCAACATGG
TGAAACCCCATCTCTACTAAAAATACAAAATTAGCTGGGCGTGGTGGCACATG
CCTGTAATCCCTGCTACTTGGGATGCTGAGACAGGAGAATCGATTGAACCTGG
GAGGCGGAGGTTGTAATAAGCCGAGATCGCACCATTGCACTCCAGCCTGGGCA
GCAAGAGCGAGACTCCGTCTCAAAAAAAAAAAAAAAAGAAATGCAGAGCTGA
AGAGCAGCCCAATGTAAATGGTCCCATTAGAGAGGAACTCTTTCATCATGGAC
ATCTGTAAGTGACCCACACTGTCCAGCAGCATCCACGATGTTTGCACAGCATA
TGGCCCCAGGGAGGGATGTCTCTAATCTGCCAGGGTCCTAGAGTTAAAGTCCT
GTGTGTTGAGTGGAGCAATTTTGCCCATTTTTTTAGTTTGGCAGAATTCATGCT
GACACTGAAACAGCTCACAGCAGACAGCATCAGGTTTCAGCATTTTTTTTTCTC
TTTTTTTAAGACAGGGTCTCACTCTGTCACCGAGGCTGGAGTGCAGTGGTGCGA
TCATAGCTCATTGCAGCCTCGACCTCTTGGGCTCAAGCAACCGTCCCCTTGCAG
AGCTTTCAGCTTGGGATCAGGTTTGGAAAACTAGAATTCGTGAATTCAGGATTT
TCTTCTGCAGTAGCTAAGCCAAGAGACTGTTTGTCTCGAGGCTAAGAGCGAAG
CGAGGCTTTCTTAAGCCCTTTAAGTCTGTCAGTTTCAGAGCCCGGGTGGACGCC
TGTAAAAAGATCCCTATCAGCTAGAAAATCATGACTCTAAGGGTCAGCTGTGT
GGTTTCACAAGGGCTCATTAGCAACTAAAAACTGCTTTTTAAAACCCTGAAAG
TGTATCTCCATTTAGAGATTGCCTCTTTTTCATGTTTTTTTCCAGAAGCTGTAAT
ATTAGCAAACACTATAGTAAGAACTATCAGTAACTGAGACTTGTTCCATTTTAT
TTACTTACTTACTTACTTATTTCTTTATTTTGAGACAGGGTCTCATTCTGTCGCC
CAGGCTGGAGCGCAGTGGCACAATCTCGCATTGCAACCTCCACCTCCTGGGTT
CAAGTGATTCTCCTGCCTCAGCCTCCCAAGTAGCTGGGTGGGATTACAGGCAC
ACACCACCACACCCAGCTAATTTTTTTTTTTTTTTTGAGACAGAGTCTTACTCTG
TTGCTCAGGCTGGAGTGCAGTGGCATGATCTCGGCTCACTGCAACCTCCACCTC
CTGGGTTCAAGCAATTCTCCTGTCTCAGCCTCCCAAGTAGATGGGACTACAGGT
GCCTGCCACCATGCCCAGCTAATTTTTGTATTTTTAGTAGAGACGGTGTTTCAC
CATATTGGTCAGGCTGGTCTCAAACTCCTGACCTCAGGTGACCCACCCGCCTCA
GCCTCCTAAAGTGCTAGGATTATAGGCATGAGCCACTGCATCCAGGCTAATTTT
TGTATTTTTAGTAGAGATAGGGTTTCACCATGTTGGCCAGGCTGGTCTCGAACA
CCTAACCTCAAGTGATCCTCCCTCCTCGGCCTCCCAAAGTGCTAGGATTACAGG
TGTGAGCCACTGGGCCCTGCCTTATCTATTTTTGATACAAGTTCTCACCCTGTTC
CCCAGGCTAGAGTGCAGTGGCGTGATCATAGCTCACTGCAACCTTTAACTCCT
GGACTCAAGTGATCCTCCCACCTCAGCCTCCTGAGTGGCTGGGGACTACAGGT
GCTCACTACCAGGCCCAGCTAGGTGAATGCTTTTCTGCACACCAGGGTATTTTC
AAGGATGCGTCCTATGAACCCATCTGATCAGTGAGCTTCTTGAGCAAGGCCTC
TGGAACCCACCTCTGCATGGTCGGGCATGAAGAACAGAGACAGTCACACCCA
GCCACTGCTGATCTGTTATCCAGTGGAGCCCAGCCTGAACAGAGAGCATCAGG
GAGGGGACTCTGGATACTGGGCCCTGCAGGCTTGCCTCCCTGCTCTCTGCCCA
GACATGTGAATGGCATCTAGGGGTGCACCCAGCATGGGACTGAGACCTACAGT
GTTGGGGACAGAGTTCCTACCTCTAGATGCCTGCCAGCCTTTGCAACCCACTGC
TGTTGACACACTTCCTTGAGTTAGTAAACATTGGCCTTTGTGCTTAGCATATAA
GCCAGGGCAGGCTCCAGGAGTGGGGGTTTGAGTTGGCCACAGCAATCAAAGT
GACAAGATGTTCCTGCTGGCCTGGAGGGGATAGGCAGGCTACACAGAGCTCTT
CTCCTCCATGGGAACCTTCTGGGCCTGTGAGATCCACCCTGGGCCACCTCCACT
CAAGTTTTCCCAGTTGGATATTTATTTATTTATTTGAGATGGAGTCTCACTCTGT
CACCCAGGCTGGAGGGCAGTGGCATGATCACTGCTCACTGCAATCTCTGCCTC
CTTGGTTCAAGTGATCCTCCTGCCTCAGTCCCCTGAGTAGCTGGGACTACAGAC
GTGCACCACCGTGCCCAGCTAATGTTTGTATTTTTAGTAGAGATGAGGTTTTAC
CATGTTGCCAGGCTGGTCTCAAACTCCTGGCCTCAAGTGATCCTCCCTCCTTGG
CCTCCCAAAGTGCTGGGATTACAGGCGTGAGCCACCACCCCCGGCCCCCAGTT
GGAAATTTAGACTTAAGGAGCTGAGGTGGACACTGGGAGCACACTGGGCAGG
CCCAGGACAGGCAGAATAAGGGGGTGGGACCACCTTGCCTGCCTCCTGAATGG
CCCAGAAAATAGAGAAAAGGAGGGAGAAGTGGTCTGCACTGGTGCAATGAGA
TCCAACTTCAGAATCAAACAGGCCAGAGTTTGGGGCTCTGCTCTTACCAGTTCC
ATGACCTGGGAGACATGTGTTCACCTCTCCAGGCCTCAGTTTCCCCATCTGATG
GGGATATCAATATCTCCCATTTGTATAGAGGGCAATCTAGCAATTTAAAGTGT
GGGTCCTGGAGTTAGAATAGCCCGGTTCATGTTCCAGTTCTTCCCTGCCGCTTG
CTGACCTCATGGCCTAGGAATGTTCTCTGTCCCAATGAATTATAACCAACACTG
AGAGTGTCCTGTGTGCCAGGTCCTGTTCCAAAGAGCGTGCTTATCTCCTTTAAT
CCCCACCACACTATTCTCATTCCTGTTTTACAAATGAGGCTCAAAGGGATATGG
TGATTTTCCCAAGTCACACTGTATGTAACAGAGCCAGGATGCAAACCTTCTCCT
GCCGTGCCTCTCAGAGCTATGGAATACAACGCTGTGACGCAAACAGGACTTTG
GGGCCAGTCTCCAAATGCCCCCTCAAATTTCCTCTAGGCAAGAGTTCTGGAGC
CGCCACTGCACTCCAGCCGGCCTTGGCTTCTGAGCTTTGGATTCTGGGCAGTAA
TGGTCACATTCCCAGCGTCCTGGAAAAGCTTGCCCTTGGCTTCTGGCCCTGACT
CTGCCTCCCACTGACTAGTGGACGTCACTGCCCCTCTCTGGGCCTGGCAACACC
ATCCTGTTCAATGAGGCTGCGGCTGGATCAGAGGTCCCCAGGCCTGGAACCTT
CCAGGGCTGGGCTCAGCTCCCCACCCCATCCCCCAGGTAGGCCAGGAGGCCTG
GGCGGGACCTGCTTGGGGTTAAATACTCCGGCCCAGCACTCCCCAGGCCTCTA
GTCCCAGTAGGAGGTTTGACTAAGATCAATCATGGTAAGTGCCATGGCCATCT
CCCCTTGGGTGGGACATCTTCAGTAACGCTTCCAGAAGCACCTTGTGTTGGAAC
CTCCAAGTCTCACTGTCCTTCTTTTTCTCCTTAGACGACTTACAGTGACAAAGG
GGCAAAGGTAAGCAGGCGAAATGTGGGAAATGTAGGGGCCTGAGTCTAGAAG
GGGGTCGGAGTGCAGGGAGACTGGGGTTTCAGCAGAGAGCAGAAGAGGAAGG
ACTGGAGTGGATACATGACGTGCATGCCTGCACACTCAGCTTTACCCCCGGGG
CTCCCCGAGACTGAGCTGGACTCAACCTCACCCTCACAACAAACTTGTATCCT
GGCCGGGCATGGTGGCTCATGCCTGTAATCCCAGTGCTTTGGGAGGCCAAGGC
AGGCAAATACTTGAGGTCAGGAGTTCGAGACCAGCCTGGTCAACATGAGGAA
ACCCCGTCTCTACTAAAAATACGAAAATTAGCCGGGCATGGTAGCGCACCCCT
GTAATCCCAGCTACTCAGGAGGCTAAGGTGGGGGAGATCACTTGAACCCTAGG
ATCGCTTGAACCTGGGAGGCGGAAGTTGCAGTGAGCCAAGATTGCACCACTGC
ACTCCACTCCAGCCTGGGCAATAGAGCAAAACTCCATCTCAGAAACAAAAACC
AAAACAAAACCTGTACCCCTTCTCAAATGCCTGCCAATCTTTTAATCCCATCTG
GAAGTTCTTTCTAAGGTCTAACTTAAATCCATATTGCTGCAACATAAGTCTATT
TCCCTGTGTGAAACCTTGGCAGGCCTCCTGAGGGGGTTTCCCCATCCAAGACA
CCCAGGAGCTCAACTTCCACATGAGAAACCTCTTTTGAGGCTTGGCAAGGCTG
CATGGGGTTCCGCTGCTCCTAAGTTCTGTGGTTGAGGCTGGAATAAGGAGGGT
GGCTAGGGGACATCACTCCACCCGCACTTTTCTAGACTCACTTTCAGGGACTCA
ACCTCACCTCTTCTCTCCTTCTGCAGCCTGAGAGAGGCCGATTCCTCCACTTCC
ACTCTGTGACCTTCTGGGTTGGCAACGCCAAGCAGGTAGAAGCCGGGGCAAGG
TGCTGGAGTGAGCAGACGGCAGGGCAGAGGGCTGCCTGACAAGGGCAGGCCT
CCAGCAGGGTCTGTGATTGGCCCTTGGGAGGGAGGATCAGGCCAGGGGTGGG
CCAGATACTCTGAATCCTAATTACACCAGCGGGAAAGTTATCTGTCAGCAAAA
TGTGCAGGCAGCAGTCTATCTTCCCAGGAGGGTCTCTGCCCCTGAGGCAATAG
GGCAGGCAGGCTGAGGGGGGCCAGGGGCTCTCTTGCGGAAGTCCCCCATGCTG
GCCCCTTGACCCTCATGAACCCTGCTGCAACCCTGTGATTCAGGCCACGTCATT
CTACTGCAGCAAGATGGGCTTTGAACCTCTAGCCTACAGGGGCCTGGAGACCG
GTTCCCGGGAGGTGGTCAGCCATGTAATCAAACAAGGGAAGGTGAGTCACCAC
CCCCAGGACAGCCCCCAACCCCTGCCTGGCCAACATGGTGAAACCCCATCTCT
ATTAAAAATAGAAAAATTAGCTGGGCATGATGGTGGGCACCTGTAATCCCAGC
TACTCAGGAGGCTGAGGCAGGCACATCGCTTGAACCCGGGAGGCAGAGGTTG
CAGTGAGCCAACGTCGTGCCACTGCACTCCAGCCTGGGCGACAGAGCAAGACT
CCATCTCAACAAAAAAAAACAAAAAACAACAAAAAACCCCAACAACAACAAC
AAAAACATATAATTCAGCTCCCACCGACCCCTCCGCTGGCATCTTTGTACAGCC
ATGCAACACGGCCCTTCCTAGTTTCGTTTCCTCCTCAGCCTGGCCCAACACTGA
CCCCACAGGTGCTCAGAAAACCTGGGATATCAGAGGAGAGGAATTTTAAGCA
GTGCCCTGACCGATGGGGTCCCTGGAGTTCTCTCCCCGAGGGAGACCTGACCT
AGCAGTTCCTCTGAGGATCCAGAGGGGGTCTGGCTGAAAGCCCCATCCCTCAG
GACCAGTGTGGGAAACTAAGGCAGAGAAGGAGCTAGAACCCAGGTCCAGGAG
TTCTTGTGCTCCATCAGGGGTGTTTCGGGTCGGCAGGGAGGGGCCGATGGGTG
CACCTCCCCATGCCCCCCTGAGCCACTAGCCTCACCTCCTTCTCCTCCTATCTTA
GATTGTGTTTGTCCTCTCCTCAGCGCTCAACCCCTGGAACAAAGGTGAGGTGCC
CGGGGAGGGTGGACGCATGGCTCTGTGGGTGGGATCTGGGTTCTGGGAGGGTG
GACGCATGGCTCCGTGGGTGGGCTCTGGGTTCAGGGCCTGGATGGAGACTAAA
AGCCAGGTGAGGGGACCTAGACTCCAGACTCCAGTGCCATCCTCTCCCTGCCG
GCCACAGAGATGGGCGATCACCTGGTGAAACACGGTGACGGAGTGAAGGACA
TTGCGTTCGAGGTGGAAGATTGTGACTACATCGTGCAGGTAAGAAAGCACCTG
GGTGGACTGAGAGAGGAAGGCCTCCGTCTGCCATGGGGACCCCATCAGTCAGG
GCTGGAAGGACACTGCTGCATCACAGTGACAAGCCCAGATGCCACATCACAGT
GACAAGCTCAGATGCTGCATCACAGTGACAAGCCCAGATGCTGCATCACAGTG
ACAAGCCCAGATGCTGGTGTGACACACACCTGGGCTCCGGGGGTGCCCTGCCT
TCCCCCGTGTCCTCTCTCTCACTGAGCCTCAGTTTCCACAGCTGCCAGATGGGG
CTAACCACAGCTGGGTTGTCAAAGGAGGAAATTAGAATTACCATTTCCTTTTTT
TTTTTTTTTTTTTTTTGTGAGACGGAGTTTTGCTCTTGTTGCCCAGGTTGGAGTG
CAATGGCACGATCTCGGCTCACCGCAACTTCTGCCTCCTGGATTCAAGCAATTC
TCCTGCCTCAGCCTCCTAAGTAGCTAGGATTACAGGCATGCGCCACCACGCCC
GGCTAGTTTTGTATTTTTAGTAGAGATGGGGTTTCTCCTTGTTGGTCAGGCTGG
TCATTTCCTTTTTTTTCTTTTTTCATTTTCTTTTTCTTTCTTTTGTGTGTGTGTGTG
TGTCAGAGTCTTGCTCTGTTGCCCAGGCTGGAGAGCAGTGGCGCAATCTTGGCT
CACTGCAACCTCCACCTCCTGGGTTCAAGCGATTCTCCTGCCTCAGCCTCCCGA
GTAGCTGGGATTACAGGCGCCTGCCACCATGCCCAGCTAATTTTTGTATTTTAG
TAGAGATGGAGTTTTGCCATATTGGCCAGGCTGGTCTTGAACTCCTGACCTCAA
GTGATCCACCCACCTCGGCCTCCTAAAGTGCTGGGATTACAGGCATGAGCCAT
TTTGCCTGGCCTCAGAATTACTGTTTCTTGAGCACAACTCTAAACCAAACACTG
TACCCAACAGGCAGCCTACAGATTCAGAGTGTGCGTTCCAGAGCCAGGCTCTC
CAGGTCTGAATCTTAGCCGTGTGACTTCGGGCAAGTGACTTAATGTCTCTGTGC
TTCAACGTCCTCACCTAGAGAATGGAGATGATAATAGTACTTTTGCATAATGTT
GCTGTGAGGATGAACGAGGTTAGAATCTAGAACAGAGCCTGGTGCATGATATA
AGCACTGTCATGCAGTCCAGTTTTGCTAAATAAATGCATGAATCTTCTTATTAT
TCTCATCCAACCATTCTTTTTACAGATGGTGAAATTGAGGACTAGAGAAGGGG
ACTGATCACAGGGGGAGTTAGCAGAGGCAGGACAGACACCCATCCCTACCTA
GTTTGTCTCCCATAGCCCCAACTCTGGGCCTGGGCCTCAGTTTCCCTATTTGCC
AAATGAGCATGAGTATTGGGGGTAATGGCTGAAGCTGGCTGGGGTTCCTACCT
GGGGCTGGAGAAACTGGAGGGGCCTGCAGGACCACGCAGAGGCCACCAGGCA
GGAGCAGCAGGCAAGGGTTCTGGAAGGCTCCAGCCCTCACCAATTCCCCGATC
CCATCTTCTCTGCAGAAAGCACGGGAACGGGGCGCCAAAATCATGCGGGAGC
CCTGGGTAGAGCAAGACAAGTTTGGGAAGGTGAAGTTTGCTGTGCTGCAGACG
GTGAGTTCCCTTTGGTGCCCCCACCCCTCCTGCTGTCGGCACCACTGAGATGCC
AGGAGCCCCGCAGCTGACTCCATCAAACCATCCCACCCATGGGCCTTGGAGCA
CAGACATTGTCCCTCAAGTCTGTCCCCCGTATTGCCTGGCCAGTGTACGGGGAC
TGCAGGAGCCACCAGCCACTCCTCCCAGGCTTCCCTTCCTCCCAGTGCGGGAG
GATGACAGCAAGGGGGTGAAGGCAGGGGCTCTGAAGAAGGGAGAAGGATGG
GGTCTGGGGACATCAGTTCCCACACGAGCTGTGTGACTTTGGGCATATTACTGT
ACCTCTCTATGACTCTGTTTAAATGAGCAAAGACTGGAACTGGAACACCTATG
TGGTCTGGGACATGCGCTTAACTTTCCTGTGCCTCAGCAGTATCTTTTTGTTTTC
CTCAAAAAAATTTTTTTTTAAAGATGGAGTCTCGCTCTGTCGCCCAGGCTGGAG
TGTAGTGGTGTGATCTTGGCTCTCTGCAACCTCCGCCTCCCAGGTTCAAGTGAC
TCTCCTGCCTCAGCCTCCCGAGTAGTTGGGACCACAAGTGCGTGCCACCATGC
CCGGCTAATTTTTTCTTTTTTGTATTTTTAGTAGAGATGGGGTTTCACCGTGTTA
GCCAGGATGGTCTCAATCTCCTGACCTGGTGATCCGCCCAACTCAGCCTCCCA
AAGTGCTGGGATTACAGGCGTGGGCCACTGTGCCCGGCCTTTTTCTTTTTTTTTT
ATTCTTTTTTCTTTTTTCAGACGGAGTCTCTCTGTCGCCCAGGCTGGAGTGCAGT
CGCGTGATCTCAGCTCACTGCAACCTCTGCCTCCTGGGCTCAAGTGATTCTTCT
GTCTCAGCCTCCTGGGGATTACAGGCATCCACCCGCACACACGGCTAATTTTTG
TGTTTTTAGTAGAGACAGGGTTTTGCCATGTTGGCCAGGCTGGTCTCGAACTCC
TGACCTCAAGTGATCCATCCGCCTCAGCCTCCCAAAGTGCTGGGATTACAGGC
GTGAGCCACCGTGCCAGGCTGTTTTCTTCAACTTTCTTTTTTCTTTTTTTTTTTTT
TTTTGAGATGGAGTCTCGCTCTGTCGCCCAGGCTGGAGTGCAGTGGCACGATCT
CGGCTCACTGCAAGCGCTGCCTTCCGGGTTCACGCCATTCTCCTGCCTCAGCCT
CCCGAGTAGCTGGGACTACAGGCGCCCGCCACCACGCCCGGCTAAATTTTGTA
TTTTTAGTAGAGGTTTCACCGTGTTATCCAGGATGGTCTCGATCTCCTGACCTT
GTGATCCGCCCGCCTCAGCCTCCCAAAGTGCTGGGATTATAAGTGTGAGCCAC
CGCGCCCCACCTCTTCAACTTTTATTATAGGTTCTGGGGTATATGTGCTGGATG
TTCAAGTTTGTTACATAGGTAAACGCATGCATGGTGGTTTGCTACACAGATCAT
CCCATCACCTAGGTATTAAGCCCGGCATCTGTTAGCTACTCTCCTTGGTGCACT
CCCTCCCCGACCTCCCCCACCTGACAGGCCCTAGTGTCTGTTGTTCCCCGTGAT
GTGTCCATCTTTTCTCATCATTTAGCTCTCACTTCTAAGTGAGAACATGCAGTG
TTTGGTTTTCTGTTCCTGCGTTAGTTTGCTGAGGATAACGGCTTCCAGCTCCATC
CATGTCCCCGTAAAGGACATGATCTTGTTCCTTTTTATGGCTGCAAGCCTCAGT
ATCTTCAACTATGACTCCTCATAGGGTCATCGTAAGGATCCAATGAGAACTGG
GCGCAGTGGCTCACGCCCATAATCCCAGCACTGTAGGAGGCTGAGGCGGGCG
AATCACTTGAGGTCAGAAGTTAGCCTGGCCAACATGGTGAAACCCCACCTCTA
CTAAAAAAAAATACAAAAGTTAGCCAGGCATGGTGGTGTGCACCTGTAGTCCC
AGCTACTTGGGAGGCTGAGGTGGGAGAATCGCTTGAACCCAGGAGGCAGAGG
TTGCAGTGAGCCAAGATTGCACCTCTGCACTCCAGCCTGGGCGACAGAACAAG
ACTCCCTCTCAAAAAAAAAAAAAAAAAAAAAAAAAAAAAAGGATCCAATGAG
GTCATTTGTGTATAGCACTTAGAACAGTGCCTGGCACATTAGACACTCAGCAA
AGATGACCAATTCTGTTTAGGACCCGTGTTACTGTAATACAGAGACAATCATA
GCTCTTACCTCCTTGAGATTAAATTCAACATAAAGGGCTGGGCACAGTGGCTC
ACACCTATAATCCCAGCAGTTTGGGAGGCTGAGGCAGGAGGATCACTTGAGGC
CAGGAGTTCGAGACCAGCATGGGCAAGATAGGGAGACCCCATCTCTACAAAA
ATTAAAAATACAAATAAAAAATAAATAGGCCAGGTGCAGTGGCTCACACCTGT
AATCTCAGCAATTTGGGAGGCCAAGGCAAGAGGACTGCTTGAGCCCAGGAATT
TGAGACCAGCCTGCGCAACAAAGTGAGACCCCATCTCTACGAAAAAATTAAAA
ATTGGCCAGGCATGATGGAGCATGCCTGTGGTCCCACCTACATGGGAGGCTGA
GGTGGGAGGATTGCTTGAGTCCAGGAGGTAGAGGCTGCAGTGAGCTGTTATCA
CACCACTGCACTTCAGCCTGGTTGACAGAGTGAGACTCTGTCTCAAAAATAAA
TGAATAGGCCGGGCGCGGTGGCTCACGCCTGTAATCCCAGCACTTTGGGAGGC
CGAGGCGGGCGGATCACGAGGTCAGGAGATCGAGACCATCCTGGCTAACACG
GTGAAACCCCGTCTCTACTAAAAAAAAAAAAAAAAAAATACAAAAAAGTAGC
CGGGCGTGGTAGCGGGCGCCTGTAGTCCCAGCTACTCGGGAGGCTGAGGCAGG
AGAATGGCGTGAACCCGGGAGGCGGAGCTTGCAGTGAGCCGAGATCGCGCCA
CTGCACTCCAGCCTGGGCGACAGAGCGAGACTCCGTCTCAAAAAAAAAAAAA
AAAAAAAAAAAAAAATAAATAAATAAATAAATGAATAAAAATGTTTTAAAAA
GTTAAAAAATTAAATTAAACATAAAAATGGTTAAAGTGTGCCTGTAATCCCAG
CACTTTGTGAGGCCGAGGCAGGTGGATCACCTGAGTTCAGGAGTTCGAGACCA
GCCTGGCCAACGTGGCAAAACCCTGTCTTTACTAAATATACAAAAATTAGCTG
GGCATGACAGGCGCCTGTAATCCCAGCTACTCGGGAGGCTGAGGCAGAAGAA
TCACTTGAACCCGGGAGACAGAGGTTGCAGTGAGCCAAGGTTGCACCACTGTA
CTCCAGCCTGGGCGACAGAGCGAGACTCCATCTCAAATAAATAAATAAAAATT
TTAAAAAATAAAAGTTAAAAAATTAAATTAAACATAAAAATGGTTAAAGTGTG
CCTGTAATCCCAGCACTTTGGGAGGCCAAGGTGGGTGGATCACTTGAGGTCAG
GAGTTCAAGACCAGCCTGGCCAAAATGGTAAAACCCCATGTCACTAAGTAAAA
CAACACAAAAATTAGCCAGGTGTGGGGCTGGGCACGGTGGCTCATGCTTGTAA
TCCCAGCACTTTGGAAGGCCAAGGGGGCGGATCACCTGAGGTTGGGAGTTTGA
GACCAGCCTGACCAACATGGAGAAACCCCATCTCTACTAAAAATACAAAATTG
GCTGGACGTGGTGGTGCATGCCTGTAATCCCAGCTACTCAGGAGGCTGAGGCA
GGAGAATCGCTTGAACCCGGGAGGCAGACGTTGTGGTGAGCCAAGATCGCAC
CATTGCACTCCAGCCTGGGCAACAAGAGCGGAGCTCTGTCTCAAAAAAAAAAA
AAAATTAGCCAGGTGTGGTGGCACGTGCTTGTAGTCCCAGCTATTTGGGAGGC
TGAGGCAGGAGAATCGCTTGAACCTGGGAGGCGGAGGTTGCAGAGAGCCAAG
ATCACACCACTACACTCTAGCCTGGCCAACAAGCAAGACTCCGTCTCAAACAA
AAAAAAAAAAAAAAAAAAAGGCTAGGTACAGTGGCTCATGCCTGTAATCCCG
GCACTTTGGGATGTTGAGGTGGGCCCGGCATGGTGGTACACGCCTATAATCCC
AGCTACTCAGGAGGCTGAGGCAGGAGAATGGCTTGAACCCGGGAGATGGAGG
TTGCAGTAAGCCGAGATCGTGCCAATGCACTCCAGCCTGAGTGACAGAGTGAG
ACTCCGCCTTAAAAAAAAAAAAAAAAAAAGATGGTTAAAGTGGCTGACAGAA
CTCTTCAGTATAATTTTTTTTTTGAGACGGAGTCTCATTCTGTCGCCCAGGCTGG
AGTGTAGTGGCACGATCTCTGCTCACTGCAAGCTCTGCCTCCTGGGTTCATGCC
ATTCTCCTGTCTCAGCCTCCCAAGTAGCTGGGACTACGGGCACCCGCCACGCC
ACCACACCCGGCTAATTTTTTGTATTTTTTAGTAGAGACAGGATTTCACCGTGT
TCGCCAGGATGGTCTCGATCTCCTGACCTCATGATCCACCCGCCTTGGCCTCCC
AAAGTGCTGGGATTACAGGCCTAAGCCACCGCGCCAGGCTGCTCTTCAGTATA
ATTTAATCCAATGCCCTCCTGCGTTCTTCATCTAGTTGACCTACTGAAATAAAT
GAAGTAGGTGCTTCATTGAGTACCTCCCCTGTGCCAGGCACTGAGCTTGGCGCT
GGGTACACCATCATGAGCAAAACAGTGTCCCCGCCCTCACGGAAACTCTCAAA
CAAGTGACAGATTGCAATTCATTTCAGAAAGGAGAAGTGACTTACCCAAGATC
ACGCATCTGGTTAGGGTCAGATGCAGGTTGGAACCCGGGGCTACGTTAGCGCT
ATGAAATGTGCTCTGTCCTCGGCGGGGATGGGTGCCCAGGGGCCGAGCCAGGC
TGTTTTCCACCCGTAGTATGGGGACACCACACACACCCTGGTGGAGAAGATGA
ACTACATCGGCCAATTCTTGCCTGGATATGAGGCCCCAGCGTTCATGGACCCC
CTACTTCCTAAACTGTGAGTGTCCCTCAGGGCAGAGGTGGGTGGAAAGCCCTG
AGGGACTGTGAAAAGTAATGCCCCTTCCTCCATGTCCACTGCGCATGTGTGTTG
GGGCAGAATGGAGCCGGCTAGGGAATTCCTGGAAGTCCTCAGGGGTGCTTGAA
GAGGTGGCACAGAGGCTGAGACCATGGCCTCTGCAACCAAACTTCCCCGGGTT
CAAATCCCAGCTCTGTTACTTCACAGCCATTTCACCCTGGGCAAGTCGCTTCAT
CTCTCTAGGCCTCAGTTTCTTCATCTGTAAAATGGGGACTGTAATAGTAAATGA
GTTGACAAAATAAAATGTCCGGCCCATTAGTAAATGCTACATAAGTGTGAGTC
CAGGCGCAGTGGCTCATACCTATAATCCCAGCACTTTGGGAGTCTGAAGCAGG
AGGTTCACTTGAGCCCCAGAGTTTTAGACCAACCTGGGCAAAAAAAAGAGTCT
CTCTTTTTGTAGAGACTCCATCTCTACAAAAAAATAGAACTCCAAAAACAGAA
GTGTGAGCTATTATTATTATTGAGGGGTCATGGAGAAGGGAGCAATCTGGGGG
GTAGCCACCTTCTCACCCCCATCTTCTTCCCTTCCCAGGCCCAAATGCAGTCTG
GAGATGATCGACCACATTGTGGGAAACCAGCCTGATCAGGAGATGGTGTCCGC
CTCCGAATGGTGAGACCTTGGCCCTCTCCCTCTGCGTGAAGATGACGGGTCCTC
ACGGTGGACTGGCCCACTCCCTGGTCCTTGCACTAACATGGAAAATACGACTG
GGTGCAGTGGCTCACACCCGTAATCCCAGCATTCTGGGAAGCCGAGGTGGGAG
GATCACTTGAGGTCAGGGGTTCAACACCAGCCTGACCAATATGGAGAAACCCC
GTCTCTACTAAAAATATAAAAACTCAGTGGGCGAGGTGTTGGGCCCCTGTAAT
TCCAGTTACTCAAGAGGCTGAGGCAGGAGAATCGCTTGAACCCGGGAGGTGG
AGGTTGGGCGACAGAGTGAGACTCCAGCCTGGGAAACAGAGCAAGATTTTGTC
TCCAATAAACAAATAAAATAAAATAGACCGGGCGCACTTGCTCACGCCTGTAA
TCCCAGCACTTTGGGAGGCGGAGGCGGGTGGATCACCTGAGGTCAGGAGTTCA
AGACCAGCCTGACCAACATGGCGAACCCCGGTCTCTACTAAAAATACAAAAAT
TAGCTGGGTGTGGCAGTGTGTGCCTGTAATCCCAGCTACTTGGGAAGCTGAGA
CAGGAGAATCGCTTGAACCAGGAAGGCGGAGGTTGCAGTGAGCTGAGATTAC
GCCACTGCACTCCAGCCTGGGTGAGAGAGAGAGACTTTGTCTCAAAATAATAA
TAATAATAATAAAGTAAAATAAAATAAAAGTGGAAAATACAGCACAAGATGT
TGGGAGTTAGCCTGAGGCCACTGATGCATTTATTCACTGTAAAACTGTCAACAT
GGAGACCAGTGGACTTGAGGCCCAAAGCGACCCACAGGCCCTCCTTCTGAGGA
ACCCTGGGGACATCACAGTCCAGGGAGGCTTGGAGGAAGGTTGCCACCACCC
AGAGAGCCCCCTGTCCAAATTCCAAGCAAGGGCCTTTTTCTCAAAGCACTTTAC
TTCCATCTGTGGGAGCCTTGCCGTCCTGTATGTATTTCGTTCCAAGACACTTCC
ACCTGAAGGAAAAATGTCATCATAAAGGTACAAATCTAGGCTGGGCGTGGTGG
CCCATATTTGGAATCTTAGTACTTTGGAGGCCCAGGCAGGTGGATGGCTTGAA
CCCAGGAGTTCAAGACAGCCTGGGCAACATAGTGAGACCCCATCTCTACAAAA
AATACGGCCGGGCACAGTGGCTCATGCCTGTAATCCCAGCACTTTGGGAGGCC
GAGGCGGGTGGATCACCTGAGGTTAGGAGTTCGAGACCAGCCTGACCAACAC
AGTGAAACACTGTCTCTACTAAATACAAAAAATTAGTTGGGCATGGTGGTGCA
TGCCTGTAATCCCAGCTACTTGGGAGGCTGAGGCAGGAGAATCGCCTGGACCC
AGGAAGTGGCAGCTGCAGCGAGCCGAGATTGCGTCACTGCATTCCAGCCTGGG
CGAAAAGAGCGAAACTCCGTCTCAAAAAACAAAAAATAAAAATACCCCAAAA
TTATCCAGGTGTGGTAGCACATGCCTGTAGTCCCAGCTACTTGGGAAGCTGAG
GTGGGAGGAGCAATTGAGCCTGGAGATTGAGGCTGCAGTGAGCCAAGACTGC
ACCACTGCACTCCAGCCTGGGCAAGAGTGAGACTCTGTCTCGAGCAACAACAA
CAACAAAAAGATCTAAATCTCTGATGGGTATGATGGCAGTGGCATCTCCTTTCT
GGAGCCTGCTCAGGGACAGATTCTGGAAGGTGGAAATGGGTGATGGAGGCCG
TCCCTGGAGGCGCTCAGAGCATATGTGTTCCACCGCCCACCCTACAGGTACCT
GAAAAACCTGCAGTTCCACCGCTTCTGGTCCGTGGATGACACGCAGGTGCACA
CGGAATATAGCTCTCTGCGATCCATTGTGGTGGCCAACTATGAAGAGTCCATC
AAGATGCCCATCAATGAGCCAGCGCCTGGCAAGAAGAAGTCCCAGATCCAGG
TGAGGCCGCCCCTGGCTGGGGGAGAGGGGAGCAGGGAAGAGGTCTGGGGGCT
CTTGTAGGGTCAGGGATGTGGGTGATGGGGCAAATGACAGTGGTCTCCTTGTT
TCCCTCCCCCAGGAATATGTGGACTATAACGGGGGCGCTGGGGTCCAGCACAT
CGCTCTCAAGACCGAAGACATCATCACAGCGGTTAGAACCCCCTTCCCTGTCC
GAATTCCTCTCTCATTCAGAGCCAGGGACGATCTCTGCAAAATTGAGAGGGCG
GGTGGCGGCAGTCCTGGGCCCGTTTCTCTGGGCTGGGAGACTGACAGGACTCC
CTCACTGCTTCTTGCAACTCATGGGCAGGCACAGCCACACCCACCCCCAGAGA
GTTCAGTCTCCACAGAACCCCTGGGCTTTGGAGGTAGCAGGCGCCAGGGTATC
AGGAAGCCTGGGTGATGGTCCCGGTTCTGCCAGTGGCTTGCTCTGTGTCTCTGG
GAGATTCAGTGCTGTCTCTGGACTTTAGTTGCCTTACCTTAAAAATCAGGAAAT
TAGGGCCGGGCACAGTGGCTCACACCTGTAATCCCAGCACTTTGGGTGGCCAA
GGCAGGGGGATCACCTGAGGTCAGGAGTTTGAGACCAGCCTGGCCAACATGGT
GAAACCCCATCTCTACTAAAAATACAAAAATTATCTGGGCGTGGTTGCGGGCA
CCTGTAATCCCAGCTTCTCAGGAGGCTGAGGCAAGAGAAGTGCTTGAACTCAG
GAGGTAGAGGTTGCAGTGAGCCAAGGTCGCACCACTGTACCACAGCCTGGGCG
ACAAGAGTGAAATTCTGCTTTAGAAAAAAATAATAGTAATTAATAAAGGAAAT
TAGGACTGGGCACAGTGGCTTATGCCTGTAGTCCCAACTACACGGGAGGCCAA
AGCAGAAGGATTGTTTGAGCCCATAAGTTAGAGATCAGCCTGGAAAACAGTGG
GACCCTGTCTCTGCAAAAAGTAAACAAAATTAACCAGGCTTGGTGGTTCATGC
CTGTAGTCCTAGCTACTTGGGAGGCTGAAGCAGGAGGATCCATTTAGCCAAGG
ATTTCAAGGCTGCAGTTAGCTATGATTGAGCCACTTTTCTCCAGCCTAGGTGAC
AGAGAGTGAGGCCCTATCTCCCCGCAACCTCAAAAAAGAAATTTTTTTTAAAA
TGAGGAAATTAAGGTTGGGCATGGTGGCTCATGCCTATAACAGTTTCTGCCCTT
GTGGAGCAATAAACAAAATAAGTAAAATATAAATGCTAGATGGTAGAGAGCA
CCCAGGAAGAAAGTAAAGCAGGGAATTGGGACAGCAAGGGTGAATGGCATTG
CACATTTAAATTGGGGGGTCAGACACTGTGGGAGGCCGAGGCAGGAGGACTG
CTTGAGCTCAGAAGTTCAAGACCAACGTGGGCAACATAGTAAGACCTCATCCT
TTTTTTTTTTTTTTTTTTTGAGACGGAGCCTGGCTCTGTCGCCCAGGCTGGAGTG
CAGTGGCGCGATCTCGGCTCACTGCAAGCTCCGCCTCCTGGGTTCACGCCATTC
TCCTGCCTCAGCCTCCCAAGTAGCTGGGACTACAGGCACCCGCCACCACGCCC
AGCTAATGTTTTTTTTTGTATTTTTAGTAGAGACGGGGTTTCACTGTGTTAGCCA
GGATGGTCTCGATCTCCTGATCTTGTGATCTGCCCACCTTGGCCTCCCAAAGTG
CTGGGATTACAGGTGTGAGCCACTGCGCCCGGCCATAAGACCTCATCTTTACA
AAAAAATCAAAAAATTAGCCAGGTGTGGTGGTGCACACCTGTGGTTCTAGTTA
CCCAGAAGTCTGAAGTAGGAGGATCAGCTGAGCCTGGGGAGGTTGAGGCTGC
AGTGAGCTAAGATCATGCCACTGCACTGCAGCCTGGGTGACAGAACAAAGACC
CTGTCTCTCAAATATATATATGTGTGTGTATATATATATATATCCTTCTTTTTTG
AGATGCAGTCTTGCTCTGTCTTCTAGGCTGGAGTGCAGTGGTGCGATTTTGGCT
CACTGCAACCTCCACCTCCTGGGTTCAAGCGATTCTCCTGCCTCAGCCTCCCAA
GTAGCTGGGATTACAGGTGCATGACACCACGCCTGGATAACTTTTTTTGTATTT
TATTTTTATTTTTTATTTTTTATTTATTTTATTTTATTTTGAGACAGAGTCTCGCT
CTGTTGCTCAGGCTAGATTGCAGTGACGCGATCTCGGCTCACTGCAAGCTCTGC
CTCCCGGGTTTACGCCATTCTCCTGCCTCAGCCTCCCAAGTAGCTGGGACTACA
CGCTCCCACCACCACGCCCAGCTAATTTTTTTTTTGTATTTTTAGTAGGGGTTTC
ACTGTGTTAGCCAGGATGGTCTTGATCTCCTAACCTCGTGATCACCCATCTTGG
CCTCCCAATGTGCTGGGATTACAGGCGTGAGCCACCTCGCCCGGCCTTTTTTTT
GTATTTTTAATAGAGACAGGATTTTGCCATGTTGGTCAGGCTGGTCTCCAACTC
CTGACTTCAAGTGATCTGCCCGCCTCAGCCTCCAAAAGTGCTGGGCTTACAGG
CGTGAGCCACCACACTCGGCCTAATAGATCCTTAATCGTTTCATGCCTCAGTTT
CCCCAGCTATGAAATGGGAGAATAGTCCTTCAGCTGCCTCAAAAGATTGCTGC
ATGGACATAATGAGGGAATACGTAGAAAGTGCTCAGGCCGGGCTCAGTGGCTC
ACGCCTGTAATCCCAGCACTTTGGGAGGCCGAGGTAGGTGGATCGCCTGAGGT
CAGGAGTTCGAGACCAACCTGGCTAACATGGTGAAACCCCATCTCTACTAAAA
ATACATAGCTGAGTGTGATGGTAGTCACCTGTAATCCCAGCTACTCAGGAGGC
TGAAGCAGGAGAATCGCTTGAACCGGGAGGCAGAGGTTGCAGTGAGCTAAGA
TCGTGCCACTGCACTCCAGCCTGGGCAACAGAGACTCCATCTCAAAAAAAAAG
AAAAAAAGAAAGTGCTCAAGGCAGGCACATGGTGAGTGTTCTATAAATCTAGC
AGTTGTTGTTCTAACGGGGCCCATGACAGAGAAGAAAGGGGAAGAGGCCACA
CAGCATCCTGGCAGTTGAGGGGAGCTAGGATAGGGACCCTGTATCCTGATGAC
CCAGCACCTCCACCTGGCTAGGGGAGTTGAACTTGGAGGCCGAAGGCTGACCT
CAGCTTTGCTCTGAAACAGATTCGCCACTTGAGAGAGAGAGGCCTGGAGTTCT
TATCTGTTCCCTCCACGTACTACAAACAACTGCGGGAGAAGCTGAAGACGGCC
AAGATCAAGGTGAAGGAGAACATTGATGCCCTGGAGGTGAGGCCCAGGCAGG
ATCCGCAGCCTTGTGGGGGGAGTATGAGCTCTGCATTGCATCTTTTCAGAAATA
TTGTCTCAGCCCGGAGGGGAGCCCAGACCAAGTCCCTGCTGGCAAAGGACATT
TATTCTAGGGTCTATGTGGGAGGAGGAGACAGATGACAAACAAATAAACAAT
AAAAGCACGGTCAGAGATAGTCAGTGCAGGAAGTTCAAACAGGCAGCCTGGG
GCTGCTTTACAGAGACTGGTCAGGGAGGGCCTCTCTGAGGAGGTGACATCTGA
GTGAAGTCTAGATCATGAGCGGGAGCTGGCCATGTGAACAGCGGGAAGAAAG
GCATTCCTGGCAGAGGGATCAGCAAGGGCCGAGGTAGGAGCATGCCCTGAGC
GTCTGAGGAACAGAAAGAATAGGCTAGAGTGGATGGGAGGCTGTTTTTGAGC
GATGACCTAGGTCAGACAGGTAAGCAAGAGCCAGTTCCTCTTGTGAGCTATTA
TTCAGGAATCTGGATTTTGTTCTTTTGTGACCAGAAACCACTGGAGGGTTTTTT
GGTTTGGTTTTAGCAGTGTTATTGACATATGTCACATAACATGTCATTTTTTTAA
GGTGTACAATTCTATGATTTCTAGCTATTTGCTATGTTGTGCAACTATCATCAA
AATCCATTTTAGGACTGGGTGTGGTGGTTCATGCTTGTAATCTCAGTACTTTGG
GAGGCCGAGGCGGGCGGATCATGAGGTCAGGAGTTCGAGACCAGCCTGACCA
ACATGGTGAAACCCTGCCTCTACTAAAAATACAAAAATTAGCCAGGCGTGGTG
ACACATGCCTGTAATCCCAGCTACTCAGGAGGCTGAGGCAGGAGAATCACTTG
AACCCGGGAGGCGGAGGTTGCAGTGAGCCGAGATTGCGCTACTACATTCCAGC
CTGGGCGACACAGTGAGACTCTGTCTCAAAAAAAAAAAAAAAAAAAAAAAAG
ATCCATTTTAGGACTAGGTATGGTGGTTCATGCCTGTAATCCCAGCACTTTGGG
AGGCCAAGGCAGGTGGATCACTTGAGGTCAGGAGTTTGAGACCAGCCTGGTCA
ATATGGCGAAACCCCCATCTCTACAAAAAGAAAAAAAAAAATTAGCTGTGCGT
GGTGATCTGTGTCTGTGGTCCCAGCTACTGGGGAGGCTGAAGTGGGAGAATCA
CTTCAACCCAGGAGGTAGAGGCTACAGTGAGCCATCATCAGGCCACTGCACTC
CAGCCTGGGTGATACCCTGTCTAAAAGAAAAACAAAAACAGTTAGGCCAGAC
ACAGTGGCTCACACCTATAATCCCAGCATTTTGGGAGGTTGAAGTGAGAGGAT
TGCTTGAGGCCAGGAGTTTAAGACCAGCCTGAGAAACACAGGGCGACCCTGTA
TCTACAAAAAAAACAGTTTTAAAAAATTAGCTGGGCATGGTGGTTCGTGCCTG
TAGTCCCAGCTACTCAGGAGGCTGAGGTGAGAGGATCACTTGAGGCCAGGAG
GTCAAAGCTGCAGTGAGCCATGATCAGGCTACTGCATTCCAGCCTGGGCAAAA
AATCAATTTTAGAACATTTTTGTCACCCCAAAAAGAAACCTTGAGCCTCTTAGC
CAGCACCTCCCAACCCCCTCTGTTCCCTTCCCCCCTCCCACCCCAGCCCTAGGC
ACCATTAATCTACCTTCAGTCTCTATAGATTTGCCTATTCTGGACATTTCATCAA
AAGGAATCACAAAGTCCAGGCACGGTGGCTCATGCCTGTAATCCCAGCACTTT
GGGAGGCTGAGGTGGGCGGATCATGAGGTCAGGAGTTCAAGACCAGCCTGGC
CAACATGGTGAAACCCTGTCTCTACTAAAAATACAAAAATTAGCTGGGTGTGA
TAGCAGATGCCTGTAATCCCAGCTACTGGGGAGCCTGAGGCCGGAGAATTGCT
TGAACCCAGGGAAGGGGAGGTTGCAGTGAGCCGAGATCGCGCCATTGCACTCC
AGCCTGGGCGACAGAGTGAAACTCCATCTCGGAAAAAAAAAAAAAGGAATCA
CAAAATAGGCCTATTGTGAGCAGCTTCTTTCCCATAGCATGTGCTCAAGGTTCA
TCTGCGTGGGGTGTGTGTCAGTGCTTCGTTCCTTTTTGTGGCTGAAAAATATCC
CATTGTATAGATAGACACACATTGTTTATCTATTCCTCGTTGATAGATATTTGG
GTTGTTTCCACTTTTGGGGTGTTATACGTAATGCTACTGTGAACATCTATGTAC
AAGGCTCTGTGTGGACGTGTTTTCAATGCACTTTGGAATATATCTAGGAGTAGC
TAGGTCACATGGTAATTCCATGTTCCCTTTTGTTTGTTTAGAGATGGAGTCTTG
CTTTGTTTCCCAGGCTGGGGTGCAGTGGTGCGATCTTGGCCCATTGCAACCTCC
AGCCTCCTGGGTTCAAGCAATTCTCCTGCCTCAGCCTCCCCAGTAGCTGGGATT
ATAGGCGCCCGCCACCACGCCCAGCTAATTTTTTTTTTTTTTTTTTTGAGACAGA
GTCTTGCTCTGTTGCCCAGGCTGGAGAGCAGTGGCCGATCTTGGCTCACTGCAA
CCTCCACTTCCCGGGTTCAAGCGATTCTCCTGCCTCTGCCTCCTGAGTAGCTGG
GATAACAGGCGTGTGCCACCATGCCCAGCCAATTTATTTTTATTTTTATTTTTA
GTAGAGATGGGGTTTCACCATGTTGGCCAGGCTGGTCTCGAACTCCTGACCTC
GGGTGATTCACTCACCTCAGCCTCCCAGAGTGCTGGGATTACAGGCGTGAGCC
ACCTTGCCCAGCCTAATTTTTGTATTTTTTTTTGTAGTGACTGGGTTTCCCCATG
TTGGCCAGGCTGGTCTCGAACTGAGCTCAGGTGATCCACCTGCCTCGGCCTCCC
AAAGTGAGCCACCATGCCTGGCCACCCCCTTTTTTTTTTTTAATCCGTATTTTAA
GGAAGCAACACTATATGTTTAACTTTTTGAGAAGCTGCCAGACTGTTTTCCAAA
GTGGTTGCACCATTTTACAGTCCCACCAGCAGTACATGAGGGTTCCGATTTCCA
CTGGAGGATTTTAAACAGGGGAATGACATGATCCGACTTTTAAAAGATCCCGC
AGCAGGTCATGGTGGCTCACGCCTGTAATCCCAACACTTTGGGAGGTCGAGGT
GGGTGGATCACCTGAGGTCAGGAGTTCGAGACCAGCCTGACCAATATCGTGAA
ACCCCGTCTTACTAAAAATACAAAAATTAGCCGGGCATGGTAGCCTGCGCCTG
TAGTCCCAGCTACTCGGGAGGCTGAGACAGGAGAATCGCTTGAACCCGGAAG
GCGGAGGTTTCAGTGAGCCAAGATTGCGCCACCGCACTCCAGCCTGGGTAACA
GCGCAAGGCTCCGTCTCAAAAAAGAAAAAGAAAGTGTCCTGATAATGTCTGCG
GGGAGGATGGATTGTAAGGATACAAGATTAGAAGCGGGCAGCAGTCTTGAAT
GGTGCCTTAGGAACTGTAGTAGGGGGATAGTAATAGGACAGTTTGGGGATATA
TTTGGAGGTGGCTGAACTGCCAGAAAAAGAGGTCATTTCCAGACTTTTGGGGT
GCAGGAGCCCCTGTGGACTTTCCAAGGATCTCACCGTGCCAGCCTCCCCTAGC
AGAGCCCCCTGTCTCCCGCCTAGGAGCTGAAAATCCTGGTGGACTACGACGAG
AAAGGCTACCTCCTGCAGATCTTCACCAAACCGGTGCAGGACCGGCCCACGCT
CTTCCTGGAAGTCATCCAGCGCCACAACCACCAGGTACTGCTTGTCCCCGGCA
GGCCCGAGGGGACAGGCAGCTAGCACACTCGGTTTTCTGGGTCCTTGGCTCAT
CTCCTCTCTTTCCCGCCACAGGGTTTTGGAGCCGGCAACTTCAACTCACTGTTC
AAGGCTTTCGAGGAGGAGCAGAACCTGCGGGGTAACCTCACCAACATGGAGA
CCAATGGGGTGGTGCCCGGCATGTAAGCCCCGCCCACCCCACGGAGGCCACAG
CCACACAGCCACGCCCCCTGATTCTGGAACTCGCCCAACTTCCCTACTGGCTGC
TCCCCTTGGGTCCCGCCCACCAGCGGACTCGGCCCCCAAGGCTCCGCCCACAC
TGACCACGCCCCTCGGCGGGGCCGCCCTCTGCTCCAGCCCTCCCGATTAAAGC
GTGCCCCGGTCCAATACTGTGTCTGTGTTTGTGTTGCGCATAAGGCAGTAGTGC
CTTATGGCAGCCAGGGGGAGGCCAGGGCGGCCGCCAGCGCCGCGGGACCGAG
GAGCTGCGGGTGCCAGCGCTTTGCCAGCGGGACCACGCGGAGAGGGGAATTCT
GGAACCAGGGAAGGAAGGGGACTTGCTCTGGAGGGAAAGACGGATGACACTG
CTTTTGTAGAGAGATAAACGCGATTCAGACAATAAAGCAAGCGATGAGCCGG
GCGTGGTGGCTCACACCTGTAATTTCAGCGCTTTGGGAGGCAGAGGTGGGAGG
ATCGCTTGAGGCCAGGAGTTCGAGCCCAGCCCGGTAACATAGCAAGACCCCGA
CCCCCCTGCGTCTACACAAATAACAAATAAAAATTAGCCTGGCGTAGTGGCGC
ACACCTGTGGTCCCAGCTACTCAGGAGGCTGAGGTGGGAGGATCGATTGATGG
ACAGAGGTCTAAGCTGCAGCGAGCCCTGATCCTGACACTGCACTCCAGCCTGG
GCAAGAGTGAGACCCTGTCTCAAAAAAAAAAAAGGCAGTTGATGAGAATATG
TGGAAGCGAAGATCGCAGGGGAGCTAATTTTGAT
SEQ ID NO: 43
CTCTAGTCCCAGTAGGAGGTTTGACTAAGATCAATCATGACGACTTACAGTGA
CAAAGGGGCAAAGCCTGAGAGAGGCCGATTCCTCCACTTCCACTCTGTGACCT
TCTGGGTTGGCAACGCCAAGCAGGCCACGTCATTCTACTGCAGCAAGATGGGC
TTTGAACCTCTAGCCTACAGGGGCCTGGAGACCGGTTCCCGGGAGGTGGTCAG
CCATGTAATCAAACAAGGGAAGATTGTGTTTGTCCTCTCCTCAGCGCTCAACCC
CTGGAACAAAGAGATGGGCGATCACCTGGTGAAACACGGTGACGGAGTGAAG
GACATTGCGTTCGAGGTGGAAGATTGTGACTACATCGTGCAGAAAGCACGGGA
ACGGGGCGCCAAAATCATGCGGGAGCCCTGGGTAGAGCAAGACAAGTTTGGG
AAGGTGAAGTTTGCTGTGCTGCAGACGTATGGGGACACCACACACACCCTGGT
GGAGAAGATGAACTACATCGGCCAATTCTTGCCTGGATATGAGGCCCCAGCGT
TCATGGACCCCCTACTTCCTAAACTGCCCAAATGCAGTCTGGAGATGATCGAC
CACATTGTGGGAAACCAGCCTGATCAGGAGATGGTGTCCGCCTCCGAATGGTA
CCTGAAAAACCTGCAGTTCCACCGCTTCTGGTCCGTGGATGACACGCAGGTGC
ACACGGAATATAGCTCTCTGCGATCCATTGTGGTGGCCAACTATGAAGAGTCC
ATCAAGATGCCCATCAATGAGCCAGCGCCTGGCAAGAAGAAGTCCCAGATCC
AGGAATATGTGGACTATAACGGGGGCGCTGGGGTCCAGCACATCGCTCTCAAG
ACCGAAGACATCATCACAGCGATTCGCCACTTGAGAGAGAGAGGCCTGGAGTT
CTTATCTGTTCCCTCCACGTACTACAAACAACTGCGGGAGAAGCTGAAGACGG
CCAAGATCAAGGTGAAGGAGAACATTGATGCCCTGGAGGAGCTGAAAATCCT
GGTGGACTACGACGAGAAAGGCTACCTCCTGCAGATCTTCACCAAACCGGTGC
AGGACCGGCCCACGCTCTTCCTGGAAGTCATCCAGCGCCACAACCACCAGGGT
TTTGGAGCCGGCAACTTCAACTCACTGTTCAAGGCTTTCGAGGAGGAGCAGAA
CCTGCGGGGTAACCTCACCAACATGGAGACCAATGGGGTGGTGCCCGGCATGT
AAGCCCCGCCCACCCCACGGAGGCCACAGCCACACAGCCACGCCCCCTGATTC
TGGAACTCGCCCAACTTCCCTACTGGCTGCTCCCCTTGGGTCCCGCCCACCAGC
GGACTCGGCCCCCAAGGCTCCGCCCACACTGACCACGCCCCTCGGCGGGGCCG
CCCTCTGCTCCAGCCCTCCCGATTAAAGCGTGCCCCGGTCCAA
SEQ ID NO: 44
MTTYSDKGAKPERGRFLHFHSVTFWVGNAKQATSFYCSKMGFEPLAYRGLETGS
REVVSHVIKQGKIVFVLSSALNPWNKEMGDHLVKHGDGVKDIAFEVEDCDYIVQ
KARERGAKIMREPWVEQDKFGKVKFAVLQTYGDTTHTLVEKMNYIGQFLPGYEA
PAFMDPLLPKLPKCSLEMIDHIVGNQPDQEMVSASEWYLKNLQFHRFWSVDDTQ
VHTEYSSLRSIVVANYEESIKMPINEPAPGKKKSQIQEYVDYNGGAGVQHIALKTE
DIITAIRHLRERGLEFLSVPSTYYKQLREKLKTAKIKVKENIDALEELKILVDYDEK
GYLLQIFTKPVQDRPTLFLEVIQRHNHQGFGAGNFNSLFKAFEEEQNLRGNLTNME
TNGVVPGM
SEQ ID NO: 45
NNANAGNCCACANANNC
SEQ ID NO: 46
UUAUAGTCCACATAUUC
SEQ ID NO: 47
[UUAU]A*G*T*C*C*A*C*A*T*[AUUC]
SEQ ID NO: 48
NNGAGAGCGANGNGCNGGAC
SEQ ID NO: 49
UUGAGAGCGATGTGCUGGAC
SEQ ID NO: 50
[UUGAG]A*G*C*G*A*T*G*T*G*C*[UGGAC]
SEQ ID NO: 51
GCNGNGANGANGNCNNC
SEQ ID NO: 52
GCUGTGATGATGTCUUC
SEQ ID NO: 53
[GCUG]T*G*A*T*G*A*T*G*T*[CUUC]
SEQ ID NO: 54
ANGNGCNGGACCCCAGC
SEQ ID NO: 55
AUGUGCTGGACCCCAGC
SEQ ID NO: 56
[AUGU]G*C*T*G*G*A*C*C*C*[CAGC]
SEQ ID NO: 57
ANGACNNCCAGGAAGAG
SEQ ID NO: 58
AUGACTTCCAGGAAGAG
SEQ ID NO: 59
[AUGA]C*T*T*C*C*A*G*G*A*[AGAG]
SEQ ID NO: 60
ANNNNGGCGCCCCGNNC
SEQ ID NO: 61
AUUUTGGCGCCCCGUUC
SEQ ID NO: 62
[AUUU]T*G*G*C*G*C*C*C*C*[GUUC]
SEQ ID NO: 63
CCAGATCCAGGAATATGTGGAC
SEQ ID NO: 64
CTCCAGGCCTCTCTCTCTCA
SEQ ID NO: 65
GCGGAAGGGTACAGCCAAT
SEQ ID NO: 66
CAGGCTGTGATACATGTGGCG

1. A method of treating a disorder of the tyrosine degradation pathway in a subject, comprising administering to the subject a composition comprising a nucleic acid silencing molecule that reduces the expression of 4-hydroxyphenylpyruvate dioxygenase (HPD).

2. A composition for use in a method of treating a disorder of the tyrosine degradation pathway in a subject, wherein the composition comprises a nucleic acid silencing molecule that reduces the expression of 4-hydroxyphenylpyruvate dioxygenase (HPD), and the method comprises administering the nucleic acid silencing molecule to the subject.

3. The method of claim 1, or composition for use of claim 2, wherein the disorder comprises a deficiency in fumarylacetoacetate hydrolase (FAH) or homogentisate 1,2-dioxygenase (HGD).

4. The method or composition for use of claim 3, wherein the deficiency results from a mutation in FAH or HGD respectively.

5. The method or composition for use of claim 4, wherein the mutation comprises one or more substitutions in, one or more insertions to, and/or one or more deletions from FAH or HGD respectively.

6. The method of any one of claims 1 and 3 to 5, or composition for use of any one of claims 2 to 5, wherein:
(a) the disorder comprises a deficiency in FAH and the disorder is Hereditary Tyrosinemia Type I (HT1); or
(b) the disorder comprises a deficiency in HGD and the disorder is Alkaptonuria (AKU).

7. The method of any one of claims 1 and 3 to 6, or the composition for use of any one of claims 2 to 6, wherein the method comprises administering to the subject a further therapeutic composition or a therapeutic regimen.

8. The method or composition for use of claim 7, wherein the further therapeutic composition comprises nitisinone (NTBC).

9. The method or composition for use of claim 7, wherein the therapeutic regimen comprises a therapeutic diet, optionally wherein the therapeutic diet comprises a tyrosine-restricted diet and/or a phenylalanine-restricted diet.

10. A nucleic acid silencing molecule that reduces the expression of 4-hydroxyphenylpyruvate dioxygenase (HPD).

11. The method of any one of claims 1 and 3 to 9; the composition for use of any one of claims 2 to 9; or the nucleic acid silencing molecule of claim 10; wherein the nucleic acid silencing molecule comprises (i) RNA, (ii) DNA, or (iii) RNA and DNA.

12. The method of any one of claims 1, 3 to 9 and 11; the composition for use of any one of claims 2 to 9 and 11; or the nucleic acid silencing molecule of claim 10 or 11; wherein the nucleic acid silencing molecule comprises or consists of an antisense oligonucleotide (AON), a small interfering RNA (siRNA), a short hairpin RNA (shRNA), a microRNA (miRNA), or a CRISPR guide RNA; optionally wherein the nucleic acid silencing molecule comprises or consists of an antisense oligonucleotide (AON) or a small interfering RNA (siRNA).

13. The method of any one of claims 1, 3 to 9, 11 and 12; the composition for use of any one of claims 2 to 9, 11 and 12; or the nucleic acid silencing molecule of any one of claims 10 to 12; wherein the nucleic acid silencing molecule comprise one or more 2′-O-methoxyethylribose (MOE) modified nucleotides or consists of 2′-O-methoxyethylribose (MOE) modified nucleotides.

14. The method of any one of claims 1, 3 to 9, and 11 to 13; the composition for use of any one of claims 2 to 9 and 11 to 13; or the nucleic acid silencing molecule of any one of claims 10 to 13; wherein the nucleic acid silencing molecule comprises one or more 2′-O-methyl (2OMe) modified nucleotides or consists of 2′-O-methyl (2OMe) modified nucleotides.

15. The method of any one of claims 1, 3 to 9, and 11 to 14; the composition for use of any one of claims 2 to 9 and 11 to 14; or the nucleic acid silencing molecule of any one of claims 10 to 14; wherein the nucleic acid silencing molecule comprises one or more locked nucleic acid (LNA) modified nucleotides or consists of locked nucleic acid (LNA) modified nucleotides.

16. The method of any one of claims 1, 3 to 9, and 11 to 15; the composition for use of any one of claims 2 to 9 and 11 to 15; or the nucleic acid silencing molecule of any one of claims 10 to 15; wherein the nucleic acid silencing molecule comprises one or more nucleoside phosphorothioates or consists of nucleoside phosphorothioates.

17. The method or composition for use of claim 16, wherein (i) the composition comprises two or more nucleic acid silencing molecules as defined in claim 16; (ii) each of the two or more nucleic acid silencing molecules are of one stereoisomer; and (iii) no further nucleic acid silencing molecules are comprised in the composition.

18. The method of any one of claims 1, 3 to 9, and 11 to 17; the composition for use of any one of claims 2 to 9, and 11 to 17; or the nucleic acid silencing molecule of any one of claims 10 to 16; wherein the nucleic acid silencing molecule comprises (a) a nucleotide sequence that has at least 75% sequence identity to a nucleotide sequence comprised in a primary transcript of HPD or (b) a nucleotide sequence that is complementary to the nucleotide sequence of (a), optionally wherein the nucleic acid silencing molecule is an antisense oligonucleotide (AON).

19. The method, composition for use, or nucleic acid silencing molecule of claim 18; wherein the nucleotide sequence comprised in the primary transcript comprises one or more of (i) a nucleotide sequence comprised in an exon, (ii) a nucleotide sequence comprised in an intron, (iii) a nucleotide sequence comprised in a 3′ untranslated region, and (iv) a nucleotide sequence comprised in a 5′ untranslated region.

20. The method of any one of claims 1, 3 to 9, and 11 to 17; the composition for use of any one of claims 2 to 9, and 11 to 17; or the nucleic acid silencing molecule of any one of claims 10 to 16; wherein the nucleic acid silencing molecule comprises (a) a nucleotide sequence that has at least 75% sequence identity to a nucleotide sequence comprised in a mRNA transcribed from HPD or (b) a nucleotide sequence that is complementary to the nucleotide sequence of (a), optionally wherein the nucleic acid silencing molecule is an antisense oligonucleotide (AON) or a small interfering RNA (siRNA).

21. The method, composition for use, or nucleic acid silencing molecule of any one of claims 18 to 20, wherein the nucleic acid silencing molecule comprises (a) a nucleotide sequence that has at least 75% sequence identity to a nucleotide sequence comprised in exon 11 of a mRNA transcribed from HPD or (b) a nucleotide sequence that is complementary to the nucleotide sequence of (a).

22. The method of any one of claims 1, 3 to 9, and 11 to 21; the composition for use of any one of claims 2 to 9, and 11 to 21; or the nucleic acid silencing molecule of any one of claims 10 to 16 and 18 to 21, wherein the nucleotide sequence comprised in the nucleic acid silencing molecule is about 10 to about 15000 nucleotides in length, optionally about 10 to about 50 nucleotides in length or about 20 to about 30 nucleotides in length.

23. The method of any one of claims 1, 3 to 9, and 11 to 22; the composition for use of any one of claims 2 to 9, and 11 to 22; or the nucleic acid silencing molecule of any one of claims 11 to 17 and 19 to 22; wherein the nucleic acid silencing molecule comprises or consists of:
(a) SEQ ID NO: 1 or a nucleotide sequence having at least 75% sequence identity thereto, optionally wherein the nucleic acid silencing molecule comprises or consists of SEQ ID NO: 2 or SEQ ID NO: 3 or a nucleotide sequence having at least 75% sequence identity thereto; or
(b) SEQ ID NO: 4 or a nucleotide sequence having at least 75% sequence identity thereto, optionally wherein the nucleic acid silencing molecule comprises or consists of SEQ ID NO: 5 or SEQ ID NO: 6 or a nucleotide sequence having at least 75% sequence identity thereto; or
(c) SEQ ID NO: 7 or a nucleotide sequence having at least 75% sequence identity thereto, optionally wherein the nucleic acid silencing molecule comprises or consists of SEQ ID NO: 8 or SEQ ID NO: 9 or a nucleotide sequence having at least 75% sequence identity thereto; or
(d) SEQ ID NO: 10 or a nucleotide sequence having at least 75% sequence identity thereto, optionally wherein the nucleic acid silencing molecule comprises or consists of SEQ ID NO: 11, SEQ ID NO: 12 or SEQ ID NO: 13 or a nucleotide sequence having at least 75% sequence identity thereto; or
(e) SEQ ID NO: 14 or a nucleotide sequence having at least 75% sequence identity thereto, optionally wherein the nucleic acid silencing molecule comprises or consists of SEQ ID NO: 15 or SEQ ID NO: 16 or a nucleotide sequence having at least 75% sequence identity thereto; or
(f) SEQ ID NO: 17 or a nucleotide sequence having at least 75% sequence identity thereto, optionally wherein the nucleic acid silencing molecule comprises or consists of SEQ ID NO: 18 or SEQ ID NO: 19 or a nucleotide sequence having at least 75% sequence identity thereto; or
(g) SEQ ID NO: 20 or a nucleotide sequence having at least 75% sequence identity thereto, optionally wherein the nucleic acid silencing molecule comprises or consists of SEQ ID NO: 21, SEQ ID NO: 22 or SEQ ID NO: 23 or a nucleotide sequence having at least 75% sequence identity thereto; or
(h) SEQ ID NO: 24 or a nucleotide sequence having at least 75% sequence identity thereto, optionally wherein the nucleic acid silencing molecule comprises or consists of SEQ ID NO: 25 or SEQ ID NO: 26 or a nucleotide sequence having at least 75% sequence identity thereto; or
(i) SEQ ID NO: 27 or a nucleotide sequence having at least 75% sequence identity thereto, optionally wherein the nucleic acid silencing molecule comprises or consists of SEQ ID NO: 28 or SEQ ID NO: 29 or a nucleotide sequence having at least 75% sequence identity thereto; or
(j) SEQ ID NO: 30 or a nucleotide sequence having at least 75% sequence identity thereto, optionally wherein the nucleic acid silencing molecule comprises or consists of SEQ ID NO: 31 or SEQ ID NO: 32 or a nucleotide sequence having at least 75% sequence identity thereto; or
(k) SEQ ID NO: 33 or a nucleotide sequence having at least 75% sequence identity thereto, optionally wherein the nucleic acid silencing molecule comprises or consists of SEQ ID NO: 34 or SEQ ID NO: 35 or a nucleotide sequence having at least 75% sequence identity thereto; or
(l) SEQ ID NO: 36 or a nucleotide sequence having at least 75% sequence identity thereto, optionally wherein the nucleic acid silencing molecule comprises or consists of SEQ ID NO: 37 or a nucleotide sequence having at least 75% sequence identity thereto; or
(m) SEQ ID NO: 38 or a nucleotide sequence having at least 75% sequence identity thereto, optionally wherein the nucleic acid silencing molecule comprises or consists of SEQ ID NO: 39 or a nucleotide sequence having at least 75% sequence identity thereto; or
(n) SEQ ID NO: 40 or a nucleotide sequence having at least 75% sequence identity thereto, optionally wherein the nucleic acid silencing molecule comprises or consists of SEQ ID NO: 41 or a nucleotide sequence having at least 75% sequence identity thereto;
(o) SEQ ID NO: 45 or a nucleotide sequence having at least 75% sequence identity thereto, optionally wherein the nucleic acid silencing molecule comprises or consists of SEQ ID NO: 46 or a nucleotide sequence having at least 75% sequence identity thereto; or
(p) SEQ ID NO: 48 or a nucleotide sequence having at least 75% sequence identity thereto, optionally wherein the nucleic acid silencing molecule comprises or consists of SEQ ID NO: 49 or a nucleotide sequence having at least 75% sequence identity thereto; or
(q) SEQ ID NO: 51 or a nucleotide sequence having at least 75% sequence identity thereto, optionally wherein the nucleic acid silencing molecule comprises or consists of SEQ ID NO: 52 or a nucleotide sequence having at least 75% sequence identity thereto; or
(r) SEQ ID NO: 54 or a nucleotide sequence having at least 75% sequence identity thereto, optionally wherein the nucleic acid silencing molecule comprises or consists of SEQ ID NO: 55 or a nucleotide sequence having at least 75% sequence identity thereto; or
(s) SEQ ID NO: 57 or a nucleotide sequence having at least 75% sequence identity thereto, optionally wherein the nucleic acid silencing molecule comprises or consists of SEQ ID NO: 58 or a nucleotide sequence having at least 75% sequence identity thereto; or
(t) SEQ ID NO: 60 or a nucleotide sequence having at least 75% sequence identity thereto, optionally wherein the nucleic acid silencing molecule comprises or consists of SEQ ID NO: 61 or a nucleotide sequence having at least 75% sequence identity thereto.

24. The method of any one of claims 1, 3 to 9, and 11 to 23; the composition for use of any one of claims 2 to 9, and 11 to 23; or the nucleic acid silencing molecule of any one of claims 11 to 17 and 19 to 23; wherein the nucleic acid silencing molecule is conjugated to a non-nucleic acid moiety.

25. The method, composition for use or nucleic acid silencing molecule of claim 24, wherein the non-nucleic acid moiety comprises N-Acetylgalactosamine (GalNAc) or a lipid nanoparticle.

26. An in vitro method for reducing expression of HPD in a cell, comprising contacting the cell with the nucleic acid silencing molecule of any one of claims 11 to 16 and 18 to 25.
